SEQUENCE LISTING (132 sequences)

SEQ ID NO: 1 - DNA, 25 bp, Homo sapiens
ggtggtggagctgtgtaagaagtac

SEQ ID NO: 2 - DNA, 22 bp, Homo sapiens
cttcctgggatggtctcatctc

SEQ ID NO: 3 - DNA, 26 bp, Homo sapiens
cagcagaccgtggtagccattgacct

SEQ ID NO: 4 - DNA, 22 bp, Homo sapiens
agacacgaacttggcctctagc

SEQ ID NO: 5 - DNA, 23 bp, Homo sapiens
ttgtaggaaacaatgatccccag

SEQ ID NO: 6 - DNA, 26 bp, Homo sapiens
ttgagggaaggtgccaaccgtgagat

SEQ ID NO: 7 - DNA, 20 bp, Homo sapiens
tcttccatgctccgtcacac

SEQ ID NO: 8 - DNA, 21 bp, Homo sapiens
cgaatctcaaagtctacgccg

SEQ ID NO: 9 - DNA, 25 bp, Homo sapiens
agccaggcccagaggatacaggaaa

SEQ ID NO: 10 - DNA, 18 bp, Homo sapiens
ttccgccgagagaacgag

SEQ ID NO: 11 - DNA, 18 bp, Homo sapiens
aagaccggcacgaagtgg

SEQ ID NO: 12 - DNA, 26 bp, Homo sapiens
tcggccctgagcaactccatcatgta

SEQ ID NO: 13 - DNA, 22 bp, Homo sapiens
tgacagtcaccacgagttcctg

SEQ ID NO: 14 - DNA, 22 bp, Homo sapiens
tctcctgttccacctcttcacc

SEQ ID NO: 15 - DNA, 26 bp, Homo sapiens
ctctgggattccgcaaaagggactat

SEQ ID NO: 16 - DNA, 22 bp, Homo sapiens
ctggctaacaatggtaccgatg

SEQ ID NO: 17 - DNA, 25 bp, Homo sapiens
gtggtctgtgcatactgtagaatgg

SEQ ID NO: 18 - DNA, 24 bp, Homo sapiens
catgaccaatgcagcagccactca

SEQ ID NO: 19 - DNA, 23 bp, Homo sapiens
cacgttggatgacacttgtgatc

SEQ ID NO: 20 - DNA, 19 bp, Homo sapiens
ctgggagatggccaattgg

SEQ ID NO: 21 - DNA, 26 bp, Homo sapiens
actaataagcagcccccccagacggt

SEQ ID NO: 22 - DNA, 24 bp, Homo sapiens
gtgtcattcagtaaagaggcgaag

SEQ ID NO: 23 - DNA, 22 bp, Homo sapiens
ctcagccctttatcattcacgc

SEQ ID NO: 24 - DNA, 26 bp, Homo sapiens
ttccggtcctggtctgcccctctata

SEQ ID NO: 25 - DNA, 21 bp, Homo sapiens
tgacggagtatgtggctacgc

SEQ ID NO: 26 - DNA, 21 bp, Homo sapiens
ccacagaccagatgtcgatgg

SEQ ID NO: 27 - DNA, 24 bp, Homo sapiens
ctggtaccgggccccagagatcat

SEQ ID NO: 28 - DNA, 20 bp, Homo sapiens
taacgttctgcaccgtgacc

SEQ ID NO: 29 - DNA, 22 bp, Homo sapiens
caggccaaagtcacagatcttg

SEQ ID NO: 30 - DNA, 26 bp, Homo sapiens
acctgctgctcaacaccacctgtgat

SEQ ID NO: 31 - DNA, 19 bp, Homo sapiens
aggcgtgctccctgatgac

SEQ ID NO: 32 - DNA, 23 bp, Homo sapiens
gctccaggtcgttcaggtagtag

SEQ ID NO: 33 - DNA, 21 bp, Homo sapiens
aggcctgctttggccgctcaa

SEQ ID NO: 34 - DNA, 21 bp, Homo sapiens
gactatgtgccgagcgatcag

SEQ ID NO: 35 - DNA, 22 bp, Homo sapiens
gtccacctggaacttggtctca

SEQ ID NO: 36 - DNA, 22 bp, Homo sapiens
ctgcttcgctgccgtgtcctga

SEQ ID NO: 37 - DNA, 22 bp, Homo sapiens
tccctggtcgaacagttttttc

SEQ ID NO: 38 - DNA, 20 bp, Homo sapiens
tttgggaggtggtcctgttg

SEQ ID NO: 39 - DNA, 29 bp, Homo sapiens
tgtaagctctcctccatccagctcctcaa

SEQ ID NO: 40 - DNA, 23 bp, Homo sapiens
gatggccctaaacagatgaagtg

SEQ ID NO: 41 - DNA, 21 bp, Homo sapiens
cctgaagcccttgctgtagtg

SEQ ID NO: 42 - DNA, 25 bp, Homo sapiens
atggcggcatccagctacgaatctc

SEQ ID NO: 43 - DNA, 22 bp, Homo sapiens
agccactcacctcttcagaacg

SEQ ID NO: 44 - DNA, 22 bp, Homo sapiens
catgtctcctttctcagggctg

SEQ ID NO: 45 - DNA, 26 bp, Homo sapiens
caaattcggtacatcctcgacggcat

SEQ ID NO: 46 - DNA, 21 bp, Homo sapiens
ctgctagccaggatccacaag

SEQ ID NO: 47 - DNA, 26 bp, Homo sapiens
ctgtgaggtaagatggtggctaatac

SEQ ID NO: 48 - DNA, 29 bp, Homo sapiens
cttgttccactgtgccttggtttctcctt

SEQ ID NO: 49 - DNA, 28 bp, Homo sapiens
gcttcgagaaagagttgagaagttaaac

SEQ ID NO: 50 - DNA, 21 bp, Homo sapiens
gacctttgccccacacatatg

SEQ ID NO: 51 - DNA, 27 bp, Homo sapiens
ctcacagaccacaagtcacagcgcctt

SEQ ID NO: 52 - DNA, 20 bp, Homo sapiens
cggcaggagctgaacaagac

SEQ ID NO: 53 - DNA, 22 bp, Homo sapiens
agcagcacacacagagccatag

SEQ ID NO: 54 - DNA, 26 bp, Homo sapiens
ccgagcgttaccagaacctgtctcca

SEQ ID NO: 55 - DNA, 22 bp, Homo sapiens
ccaacacccgtacatcaatgtc

SEQ ID NO: 56 - DNA, 26 bp, Homo sapiens
cactcttctattgtgtgttccctttc

SEQ ID NO: 57 - DNA, 28 bp, Homo sapiens
caccaccaaagatccctgacaagcagtt

SEQ ID NO: 58 - DNA, 17 bp, Homo sapiens
gccaggcaggcatttcc

SEQ ID NO: 59 - DNA, 20 bp, Homo sapiens
atgcttcgcctctgcttcac

SEQ ID NO: 60 - DNA, 27 bp, Homo sapiens
tcagccaccatctgccttgcttacctt

SEQ ID NO: 61 - DNA, 20 bp, Homo sapiens
agcccagaggaagggacaac

SEQ ID NO: 62 - DNA, 20 bp, Homo sapiens
tgtgagcgctcgtgagattg

SEQ ID NO: 63 - DNA, 27 bp, Homo sapiens
ctcctgcaaaagaaccctcggtcaaca

SEQ ID NO: 64 - DNA, 21 bp, Homo sapiens
ccatgtaggaagcggctgtac

SEQ ID NO: 65 - DNA, 20 bp, Homo sapiens
tcagcccccatgtcaaaaac

SEQ ID NO: 66 - DNA, 27 bp, Homo sapiens
atcctgagaccttcgtgcaggcaatct

SEQ ID NO: 67 - DNA, 22 bp, Homo sapiens
gctgtcgctcccatatttatcc

SEQ ID NO: 68 - DNA, 22 bp, Homo sapiens
cacaatggactcgcacttcttc

SEQ ID NO: 69 - DNA, 28 bp, Homo sapiens
ctgtcagccctgtgtggtcaacgaatac

SEQ ID NO: 70 - DNA, 20 bp, Homo sapiens
ctggtctggaaagagctggg

SEQ ID NO: 71 - DNA, 20 bp, Homo sapiens
cagcaggagatccaccaagg

SEQ ID NO: 72 - DNA, 23 bp, Homo sapiens
ccccatcttctttggtgcccgac

SEQ ID NO: 73 - DNA, 24 bp, Homo sapiens
gggaatatgactacgtgaccaatg

SEQ ID NO: 74 - DNA, 24 bp, Homo sapiens
ggatccctgaagtcaatgttgatc

SEQ ID NO: 75 - DNA, 26 bp, Homo sapiens
cattcaagacgaatcgccagtctccc

SEQ ID NO: 76 - DNA, 21 bp, Homo sapiens
gattggaagacccgtttgagc

SEQ ID NO: 77 - DNA, 22 bp, Homo sapiens
caggagaaggcttgatgaaagc

SEQ ID NO: 78 - DNA, 22 bp, Homo sapiens
ctgggaagcccaaaaccggcaa

SEQ ID NO: 79 - DNA, 21 bp, Homo sapiens
aggagttccctggatttttgg

SEQ ID NO: 80 - DNA, 22 bp, Homo sapiens
gcccactttgccatctctacac

SEQ ID NO: 81 - DNA, 28 bp, Homo sapiens
caaaaagaccctctggctgtggacaaaa

SEQ ID NO: 82 - DNA, 23 bp, Homo sapiens
catggctgagatgaggaatgaag

SEQ ID NO: 83 - DNA, 19 bp, Homo sapiens
gctggcatgttggctatcg

SEQ ID NO: 84 - DNA, 25 bp, Homo sapiens
acgcaggtcccagcctcctcttcat

SEQ ID NO: 85 - DNA, 23 bp, Homo sapiens
tggattcgtcaatgatgcctatc

SEQ ID NO: 86 - DNA, 18 bp, Homo sapiens
atgccacatccgcaatgg

SEQ ID NO: 87 - DNA, 24 bp, Homo sapiens
agacctgcggcacgtgtccgtcta

SEQ ID NO: 88 - DNA, 1566 bp, Homo sapiens
ggcccgttaagaagagcgtggccggccgcggccaccgctggccccagggaaagccgagcggccaccgagccggcagagacccaccgagcggcggcggagggagcagcgccggggcgcacgagggcaccatggcccagacgcccgccttcgacaagcccaaagtagaactgcatgtccacctagacggatccatcaagcctgaaaccatcttatactatggcaggaggagagggatcgccctcccagctaacacagcagaggggctgctgaacgtcattggcatggacaagccgctcacccttccagacttcctggccaagtttgactactacatgcctgctatcgcgggctgccgggaggctatcaaaaggatcgcctatgagtttgtagagatgaaggccaaagagggcgtggtgtatgtggaggtgcggtacagtccgcacctgctggccaactccaaagtggagccaatcccctggaaccaggctgaaggggacctcaccccagacgaggtggtggccctagtgggccagggcctgcaggagggggagcgagacttcggggtcaaggcccggtccatcctgtgctgcatgcgccaccagcccaactggtcccccaaggtggtggagctgtgtaagaagtaccagcagcagaccgtggtagccattgacctggctggagatgagaccatcccaggaagcagcctcttgcctggacatgtccaggcctaccaggaggctgtgaagagcggcattcaccgtactgtccacgccggggaggtgggctcggccgaagtagtaaaagaggctgtggacatactcaagacagagcggctgggacacggctaccacaccctggaagaccaggccctttataacaggctgcggcaggaaaacatgcacttcgagatctgcccctggtccagctacctcactggtgcctggaagccggacacggagcatgcagtcattcggctcaaaaatgaccaggctaactactcgctcaacacagatgacccgctcatcttcaagtccaccctggacactgattaccagatgaccaaacgggacatgggctttactgaagaggagtttaaaaggctgaacatcaatgcggccaaatctagtttcctcccagaagatgaaaagagggagcttctcgacctgctctataaagcctatgggatgccaccttcagcctctgcagggcagaacctctgaagacgccactcctccaagccttcaccctgtggagtcaccccaactctgtggggctgagcaacatttttacatttattccttccaagaagaccatgatctcaatagtcagttactgatgctcctgaaccctatgtgtccatttctgcacacacgtatacctcggcatggccgcgtcacttctctgattatgtgccctggccagggaccagcgcccttgcacatgggcatggttgaatctgaaaccctccttctgtggcaacttgtactgaaaatctggtgctcaataaagaagcccatggctggtggcatgcaaaaaaaaaaaaaaaaaaaaaaaaaaaaaaaaaaa

SEQ ID NO: 89 - DNA, 1254 bp, Homo sapiens
atgggcgacaaagggacgcgggtgttcaagaaggccagtccaaatggaaagctcaccgtctacctgggaaagcgggactttgtggaccacatcgacctcgtggaccctgtggatggtgtggtcctggtggatcctgagtatctcaaagagcggagagtctatgtgacgctgacctgcgccttccgctatggccgggaggacctggatgtcctgggcctgacctttcgcaaggacctgtttgtggccaacgtacagtcgttcccaccggcccccgaggacaagaagcccctgacgcggctgcaggaacgcctcatcaagaagctgggcgagcacgcttaccctttcacctttgagatccctccaaaccttccatgttctgtgacactgcagccggggcccgaagacacggggaaggcttgcggtgtggactatgaagccaaagccttctgcgcggagaatttggaggagaagatccacaagcggaattctgtgggtctggtcatccggaaggttcagtatgccccagagaggcctggcccccagcccacagccgagaccaccaggcagttcctcatgtcggacaagcccttgcacctagaagcctctctggataaggagatctattaccatggagaacccatcagcgtcaacgtccacgtcaccaacaacaccaacaagacggtggagaagatcaagatctcagtgcgccagtatgcagacatctgccttttcaacacagctcagtacaagtgccctgttgccatggaagaggctgatgacactgtggcacccagctcgacgttctgcaaggtctacacactgacccccttcctagccaataaccgagagaagcggggcctcgccttggacgggaagctcaagcacgaagacacgaacttggcctctagcaccctgttgagggaaggtgccaaccgtgagatcctggggatcattgtttcctacaaagtgaaagtgaagctggtggtgtctcggggcggcctgttgggagatcttgcatccagcgacgtggccgtggaactgcccttcaccctaatgcaccccaagcccaaagaggaacccccgcatcgggaagttccagagaacgagacgccagtagataccaatctcatagaacttgacacaaatgatgacgacattgtatttgaggactttgctcgccagagactgaaaggcatggaggatgacaaggaggaagaggaggatggtaccggctctccgcggctcaacgacaga

SEQ ID NO: 90 - DNA, 1770 bp, Homo sapiens
tggcagcgggcgaggaggctgcgagcgagccgcgaaccgagcgggcggcgggcgcgcgcaccatgggggagaaacccgggaccagggtcttcaagaagtcgagccctaactgcaagctcaccgtgtacttgggcaagcgggacttcgtagatcacctggacaaagtggaccctgtagatggcgtggtgcttgtggaccctgactacctgaaggaccgcaaagtgtttgtgaccctcacctgcgccttccgctatggccgtgaagacctggatgtgctgggcttgtccttccgcaaagacctgttcatcgccacctaccaggccttccccccggtgcccaacccaccccggccccccacccgcctgcaggaccggctgctgaggaagctgggccagcatgcccaccccttcttcttcaccataccccagaatcttccatgctccgtcacactgcagccaggcccagaggatacaggaaaggcctgcggcgtagactttgagattcgagccttctgtgctaaatcactagaagagaaaagccacaaaaggaactctgtgcggctggtgatccgaaaggtgcagttcgccccggagaaacccggcccccagccttcagccgaaaccacacgccacttcctcatgtctgaccggtccctgcacctcgaggcttccctggacaaggagctgtactaccatggggagcccctcaatgtaaatgtccacgtcaccaacaactccaccaagaccgtcaagaagatcaaagtctctgtgagacagtacgccgacatctgcctcttcagcaccgcccagtacaagtgtcctgtggctcaactcgaacaagatgaccaggtatctcccagctccacattctgtaaggtgtacaccataaccccactgctcagtgacaaccgggagaagcggggtctcgccctggatgggaaactcaagcacgaggacaccaacctggcttccagcaccatcgtgaaggagggtgccaacaaggaggtgctgggaatcctggtgtcctacagggtcaaggtgaagctggtggtgtctcgaggcggggatgtctctgtggagctgccttttgttcttatgcaccccaagccccacgaccacatccccctccccagaccccagtcagccgctccggagacagatgtccctgtggacaccaacctcattgaatttgataccaactatgccacagatgatgacattgtgtttgaggactttgcccggcttcggctgaaggggatgaaggatgacgactatgatgatcaactctgctaggaagcggggtgggaagaagggaggggatggggttgggagaggtgagggcaggattaagatccccactgtcaatgggggattgtcccagcccctcttcccttcccctcacctggaagcttcttcaaccaatcccttcacactctctcccccatccccccaagatacacactggaccctctcttgctgaatgtgggcattaattttttgactgcagctctgcttctccagccccgccgtgggtggcaagctgtgttcatacctaaattttctggaaggggacagtgaaaagaggagtgacaggagggaaagggggagacaaaactcctactctcaacctcacaccaacacctcccattatcactctctctgcccccattccttcaagaggagaccctttggggacaaggccgtttctttgtttctgagcataaagaagaaaataaatcttttactaagcatgaaaaaaaaaaaaaaaaaa

SEQ ID NO: 91 - DNA, 1975 bp, Homo sapiens
actttcccccctcggcgccccaccggctcccgcgcgcctcccctcgcgcccgagcttcgagccaagcagcgtcctggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattttcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagagcctcgaggaggtaatgaattaaagaagagaactgcctttggcagagttctataatgtaaacaatatcagactttttttttttataatcaagcctaaaattgtatagacctaaaataaaatgaagtggtgagcttaaccctggaaaatgaatccctctatctctaaagaaaatctctgtgaaacccctacgtggaggcggaattgctctcccagcccttgcattgcagaggggcccatgaaagaggacaggctacccctttacaaatagaatttgagcatcagtgaggttaaactaaggccctcttgaatctctgaatttgagatacaaacatgttcctgggatcactgatgactttttatactttgtaaagacaattgttggagagcccctcacacagccctggcctccgctcaactagcagatacagggatgaggcagacctgactctcttaaggaggctgagagcccaaactgctgtcccaaacatgcacttccttgcttaaggtatggtacaagcaatgcctgcccattggagagaaaaaacttaagtagataaggaaataagaaccactcataattcttcaccttaggaataatctcctgttaatatggtgtaca

SEQ ID NO: 92 - DNA, 1411 bp, Homo sapiens
gcgactgtctccgccgagcccccggggccaggtgtcccgggcgcgccacgatgcggccgcggctgtggctcctcctggccgcgcagctgacagttctccatggcaactcagtcctccagcagacccctgcatacataaaggtgcaaaccaacaagatggtgatgctgtcctgcgaggctaaaatctccctcagtaacatgcgcatctactggctgagacagcgccaggcaccgagcagtgacagtcaccacgagttcctggccctctgggattccgcaaaagggactatccacggtgaagaggtggaacaggagaagatagctgtgtttcgggatgcaagccggttcattctcaatctcacaagcgtgaagccggaagacagtggcatctacttctgcatgatcgtcgggagccccgagctgaccttcgggaagggaactcagctgagtgtggttgatttccttcccaccactgcccagcccaccaagaagtccaccctcaagaagagagtgtgccggttacccaggccagagacccagaagggcccactttgtagccccatcacccttggcctgctggtggctggcgtcctggttctgctggtttccctgggagtggccatccacctgtgctgccggcggaggagagcccggcttcgtttcatgaaacaattttacaaataagcagagaatacggttttggtgtcctgctacaaaaagacatcggtcagtaatgagcacgatgtggaaaaatgagagaagggacacattcaaccctggagagttcaatggctgctgaagctgcctgcttttcactgctgcaaggcctttctgtgtgtgatgtgcatgggagcaacttgttcgtgggtcatcgggaatactagggagaaggtttcattgcccccagggcacttcacagagtgtgctggaggactgagtaagaaatgctgcccatgccaccgcttccggctcctgtgctttccctgaactgggacctttagtggtggccatttagccaccatctttgcaggttgctttgccctggtagggcagtaacattgggtcctgggtctttcatggggtgatgctgggctggctccctcttggtcttcccaggctggggctgaccttcctcgcagagaggccaggtgcaggttgggaatgaggcttgctgagaggggctgtccagttcccagaaggcatatcagtctctgagggcttcctttggggccgggaacttgcgggtttgaggataggagttcacttcatcttctcagctcccatttctactcttaagtttctccccatttctactcttaagtttctcagctcccatttctactctcccatggcttcatgcttctttcatttttctgtttgttttatacaaatgtgttagttgtacaaataaa

SEQ ID NO: 93 - DNA, 9794 bp, Homo sapiens
tgtttccgtgcgcggccgctgcgcactcggcactgggcggcgctggctggctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaataaaaaaaatgtctcatttgggaaggaaattccttttaaaaaagagttgagacacttagaaaactaatgttttatatttagtcaagagttatttaagaaagtcaagcttgtttaacaacaaaatatgaagatttaagtgttaattgctggatccattttaaaataagattttaattaacatttgtaaatggtatattttcgtttgtaacaaaccattgtcttttttcaaggatgaacagagtttatgaaggagcatcattctaagaattaagtgatgtagtctttatgtttggacagttcaccagattctcaagaaggctttcaaacaactataaagtttgatgtttgtcctgctgagctaatggggaaagttatagcataaaaattgtgtaaccgcatagatatgtcatttttaaaaactggtttaacagaaatcaagcaaagtcacaaatatgttcacaagttggaattatttattgagtcaaaatgtcgaatcgaacattttgaatgaagtaagtgttataaatgaaaaattgcctgatgtttagcagtttgtattctctaaagctttttttcaaaagttcaggctttctacttactgggaagttggtggtcctcttagtccctgataaatcaaggcaatcacattcatgtgagctggatgaatttataagttataaagaccttatccttcataccttgaggatgattgcactggttttgaagtcagttgcttaatgatgaggtgagaaatgtatcctgttgctaaatctgtcttagacccttggtgaaacttgaagatttcagtttataaagataaaatcaagcatcttttgtgcagttttctttttttaatgcaagaatggtggggaggtttgtttgtaagcatgaaactttgagaatctttattaagaaaatgacataatttttaaaaaccttgtagccaagaacatatgtggccacattaccagtaataaatgtttttctctttatattggccaaaagggaataaaaatgtcatcataggaatttgtacatatgctactgatttgcctagaaaatagcaagtttgatattgctcactttgcaaatatagggccatgtggcacttttatctataggacagattaataaaaatgaagtggggaggggtttatttttgatatattactcttatgagttttcaagctttgataatgtttaactgaaaagtggcttagaaagggctagatccaatgtgttcattattaaataattgctatcagatacaattttaagttcattctttttcaaactcaagtaccatattggcaaccataatattgtcataggtgctctcttcatttagatattcttggggggggtggcatttgtataatatatgtgtacatatatatatatatatatatatatacatacagtatataatctaaagctctgagagctcttaagtcaggaatgctgagtattatagtatattgaggtcagatgaaattttacatttttgtgtgttctgttgcattccttctggtagtttctatgactgcattactccagcactcatgattgattttatcttctaattttcttccaagtattttattttttattagttttctttggcttgatacttttaaatatgttactagtcacttgaaagcctctcccccaaaagtatttggtttgtatgctttgtctgtggcagctataacagtggtaagaacattttgaagatagctttttaaaggaaccactgattttttcaaaaatcatcctgggggaggaattttggcatttcatttgagcagggattttgtcagaaaatgtgttttgatggtaggtcagcagcagtgctagtctctgaaagcacaataccagtcaggcagcctatcccatcagatgtcatctggctgaagtttatctctgtctctcaggataaatccctgtaggacaaatccctactatcatttctaccttttggggtgacatgtggaatcatacaaaggcttaggaagaaatacgtttgtttaaaccaggatgctttacttacttgaagtgacttcaatctagatttcttttaatatttaacaaatttttaattctatgatcagccacagtcagctattaccataaattggtctctgtttattttgaagatcacggctgcttcattttgcaggattaagtagggctaatgtatcttaaagttaagatcttgaattaaagtgagttttagaaatagtgttacataccttttcagttgttttcaagaggctttatttttgttgcctttgtagccctgaaagctgttggtatattttttccctcatggacccaatagaaaagttgtatatttatttggattatatttacattctgtcctttgtaaatgtttggtgtaacttgcacttttttaaatgacccagtttgggtattagcaacttaagaaattccctcatcaagtaattctcaactttttagtctttctcctctcttcaaatcatgtgactttttaaatggaagtttttcattgattaaaatattttagcacctaaaagctagccttaaaaacagctgtaaaagaaaaacatcaggaaattagatatgactagcccagttaattaaaagacgggctcaaaccttgttttattctttttcatcttggatgaagattgaagggaaaataactcaagtgcataatatttattttcaatttttaatgagactttatcctcatcacaacattaatactgtacatagtatgccaaaatatccattaatttgtctagaatagtacaagactttttaaagcaattgtcctcacagagaccacatgtaatatactgaaatatgttcatttttaatggctttgttaacatcaaagaaatgctgcctaaatttgatttcagatgaggaaggagaaagtaaagtgtgcatagtaaggctgtaggtgaagagttgtgagataaatagttcactcagttgtacaaagcacaactagaactttttgttgggaggcttacatacatcttgaatattcttaatgtaataatgttgactattaagttggctacacagtcactgtatgtactaggaactggtttccttgacattctagaatcaatggctaggagaggcattaatctttgaggggctgaacatatcatgaagctgagtcagtatggaaaattttcaaataaacagggtgctgaagttccatctgtctcatctgcttatgataagttcttattgattagtgaatgtagcttaagcctttgtatgtgtcctcagggggcagaccgactttaagagggaccagataacgtttgaatggagggattatatttcaggtgttttagcttgaaatttattttttaaaaaaagaaaaatttaaaaaatatataaataaaatagaacaaagccggtgatgcaagttgatattataaacaggcagttttagcacagaaagaaaatactgacctgtctgcattctggtacggtgggtgcaggtcccagctgggtatgacatgatacatttttaattattctcaccagcaagtaaaaggaaaatgaacaatcttttggaattgtctttgaaaaggatcaaagagtaggaaattcacatttgacctaacattacttgcctatagaagtatggcatttccaagcttttgtctgaggagcatctcagagaagtgagagtaaatctgagttagcttaaaaattggtagggaggaagaaaatctctgcaaataatgattttatgtttgttggccaagtgaaatgatctatcattgtgtttgggaggttttattttcttatgtttttaaaattggtaaatgctttatagatgtatttttatccaagtgccactccaatttgtgtatgtaataaaattatttatattaaaagtgggaaataattgtcaacattttttttgagtatagatttattaggggtggcaaagaagagtgctagttagcagttttccatgtaaagttgtccttgactgatttgtccacatgtcagttgtaactcccccactccctgcaaaaggaattatttctaacccagatgtatcacttgaaactttttagaagcaaaataatcagggaagttcctagaaaggtgtttggctttttggtttttgagggttggggtaaagaagacttcccccacaactgtcagcacaaaacagggtattgatttttaactctgatgtttctattggagttgaatactaaataaataactataatgagggaaatacatttctaataaaattccctacattctagaaacatccctgttttaatttttttatctaaatctttttgtgctttatgtgtaaagaaaaaaatgtactgagttacaatgcattttattaacactatgtacataatagctgctttgtgttcagaatagtagcagttgctttgtatattaaagtgatccttgtgaatttgtgaaatattgtcataaagtgctttttcttactgtaatctttgtggtatcaactgtcataatgctctttttacacaaacatttatgtgcagtcacataaacatgcttttaaaaactctgtaagtctcttttttggggatgggatctctatattttgttgggttttttttgctagtagtgtgaagccatgttttattggacttaaagttacaatatattacaagcttgtgttggaaggcagcaaaactaattcagacaacaacatgtcttcagttactggatccctaattttcaggacaaaacctgtttttcaataagattgaacagtgcctatttgtggatttggagatgttactgtcaagatgactaatggagacatacgaccagctgtgtctgatgtcataaaacacgtgttcactgaaaggacaataagactatataccttctcaggtccccttgcaattctaaaactctgtgatcatataaattggaaggaaaggggaggggatatggttaatctttgcttaagctgtaagaataaaaaagttatctcctatactattaacttctgaaataagttctgagacgagacatctgaaaataagcagctgcattatttgtatgtttcttcactgccaagatgtgttcaagcctgctatacctgccattgtattggaaggcttaatgaatttcatttattttctgcaacaacgattacagaatttattgcacaaaatgagacattttgagagtgatattaattacatgagggacaataggcatgaactaggattgttctaagcaaatcggaatcgggtcaccctgccacgttcaggtgcttggaccttcaggaaaagattgcccatcttgtcatttgaccaggcactgaagtgacaagaccatccttgagaagtcacatccaaagataaaattctgatccatttctagttttagtgtttcgccactgaagacttaacatatgtcttttacactcaggttgcaaaacacaggcccaagacaaacttaacttctcccccaaatcttccttccgctggtttttccatctcgtaagtggtgccactatccatctgttaaattgtttaggggaaacctagaaaagcactaccttaatcagtgttatccttcttcttaactgtgcgtcctaatttctccacatctttcttaagtgcagtgaccaaaccggatgagaattctaacacgggcctgacatcaaatggaaaggaaggataatgtccaggagttggaatgttatccttgtttttaattaagatgcaattcacataaattaactttttaagtgaacaattaagtggtagtacatccacaatggtgtacaaccaccacttctatctagctccaaaacattctcatcactccaaaagtaaagtcccgttactctccattttctcctcccaccgcccttgtccctggcaaccaccaatctgcttcctgtttctttggatttacatccgggtatttcatgtgagactcatacactgtgtattacttctttcgtctagctttaatgtgttgttgaggttgatccattgtaacatgttatcactacttcattcctttttatagctaagtatactttttatagtaagtatgccattgtagatatataccacaagtttatcgattcatccagttgagttgtttctactgtttggctaatgttcatagtgctgttatgaatgttcgtgtacaagtatttgagtccgtgttttcaattatttggggtatatgcctgggagtggagttgctgggtcatgttgaaatcgcacatttaactttttgaggaactgtcaaactttccctcagcagctgtaccgttttaccttccaccattgatgtatgagggttccaatttctccacaccttcaccaacacttattttgccattttaaaaattatagccatcctcatgggtgtggtctctcattgtggttttgatttgcatttccctgattactaatgatgtggagcatcttttgttgtctttggccatctgcgtatcttctttgaagaaatgtctgttgaggtcctttgttcattgaaattttgttgttgggttctgagttccttatatattctgggtactaggcccttataatattttcgcctataagtttttgctttataatgtcctcattgttttcaaacttactttatgtaatatgtacacttctaaaaaaaagaaacatggaaaagggcaaactgtaaaaaaaaaaaaaaaaa

SEQ ID NO: 94 - DNA, 1241 bp, Homo sapiens
gaattcgcggccgccgcttctcacggcattcagcagcagcgttgctgtaaccgacaaagacaccttcgaattaagcacattcctcgattccagcaaagcaccgcaacatgaccgaaatgagcttcctgagcagcgaggtgttggtgggggacttgatgtcccccttcgacccgtcgggtttgggggctgaagaaagcctaggtctcttagatgattacctggaggtggccaagcacttcaaacctcatgggttctccagcgacaaggctaaggcgggctcctccgaatggctggctgtggatgggttggtcagtccctccaacaacagcaaggaggatgccttctccgggacagattggatgttggagaaaatggatttgaaggagttcgacttggatgccctgttgggtatagatgacctggaaaccatgccagatgaccttctgaccacgttggatgacacttgtgatctctttgcccccctagtccaggagactaataagcagcccccccagacggtgaacccaattggccatctcccagaaagtttaacaaaacccgaccaggttgcccccttcaccttcttacaacctcttcccctttccccaggggtcctgtcctccactccagatcattcctttagtttagagctgggcagtgaagtggatatcactgaaggagataggaagccagactacactgcttacgttgccatgatccctcagtgcataaaggaggaagacaccccttcagataatgatagtggcatctgtatgagcccagagtcctatctggggtctcctcagcacagcccctctaccaggggctctccaaataggagcctcccatctccaggtgttctctgtgggtctgcccgtcccaaaccttacgatcctcctggagagaagatggtagcagcaaaagtaaagggtgagaaactggataagaagctgaaaaaaatggagcaaaacaagacagcagccactaggtaccgccagaagaagagggcggagcaggaggctcttactggtgagtgcaaagagctggaaaagaagaacgaggctctaaaagagagggcggattccctggccaaggagatccagtacctgaaagatttgatagaagaggtccgcaaggcaagggggaagaaaagggtcccctagttgaggatagtcaggagcgtcaatgtgcttgtacatagagtgctgtagctgtgtgttccaataaattattttgtaggg

SEQ ID NO: 95 - DNA, 2924 bp, Homo sapiens
gacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcaggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgaccaggctactgtcagctcccctcggagaaaactgtgcagtctgcgtgtgaacagctcttctcctttagagcacaatggatctcgagggatcttccatacctaccagttctgcgcctcgaggccgcgactctaga

SEQ ID NO: 96 - DNA, 1745 bp, Homo sapiens
ccccgtagaaccgagggggtgggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggcttctcctccccacccgccctccccacgggcctcgggacctcaggtgggcccagttcaatctcccgctgctgctgctgcgcccttaccttccccagcgtcccagtctctggcagttttggaatggaagggttctggctgccccaacctgctgaagggcagaggtggagggtggggggcgctgagtagggactcacggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggctgcccctgccacctccctgacccgtctaatatataaatatagagatgtgtctatggctgaaaaaaaaaa

SEQ ID NO: 97 - DNA, 1611 bp, Homo sapiens
acataatttctggagccctgtaccaacgtgtggccacatattctgtcaggaaccctgtgtgatcatggtctggatctgcaacacgggccaggccaaagtcacagatcttgagatcacaggtggtgttgagcagcaggcaggcaggcaatcggtccgagtggctgtcggctcttcagctctccgctcggcgtcttccttcctctcccggtcagcgtcggcggctgcaccggcggcgggcagtcctgcgggaggggcgacaagagctgaggcgcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttccaacaggcccatctttccagggaagcattatcttgaccagctgaatcacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctaaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcactggcc

SEQ ID NO: 98 - DNA, 1702 bp, Homo sapiens
ccggcagtcccgagtgcttcccgcagagggctggtggtgggagcggagtggagtcgggcggggccgaagccgggccgtgggcgtagatgggggccgggcggcggcggagcggcggaacgcgggatgggctgcaccgtgagcgccgaggacaaggcggcggccgagcgctctaagatgatcgacaagaacctgcgggaggacggagagaaggcggcgcgggaggtgaagttgctgctgttgggtgctggggagtcagggaagagcaccatcgtcaagcagatgaagatcatccacgaggatggctactccgaggaggaatgccggcagtaccgggcggttgtctacagcaacaccatccagtccatcatggccattgtcaaagccatgggaaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctgaggggcagcggggcctggcgggatgggccaccgccgaatttgtaccccccaacccctgaggaagatgggggcaagaagatcacgctccccgcctgttcccccgccgcttttctcctctttcctctctttgttctcagctccccctgtcccctcagctccaaacgtaggggaggggttcgcacaggcctccctgtttgaagcctgcccttgtctgagatgctggtaatggccatggtacccccttctgggcatctgttctggtttttaaccattgtcttgttctgtgatgaggggaggggggcacatgctgagtctcccaaggctgcgtctggaggggcccctgcttctccagcctggacccccagctttgcccaacaccagcccctgccccagcccaagtccaaatgtttacgggagcctcctgcccagtcccccaaccccagccgctcggaggccccaaaggaaaaagcacaagaagcgtgagacgccaccattcctggaaaccacagtcc

SEQ ID NO: 99 - DNA, 1185 bp, Homo sapiens
atgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 100 - DNA, 4788 bp, Homo sapiens
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 101 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 102 - DNA, 1128 bp, Homo sapiens
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtccagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatattttatatttttaagaagtaccacttgaaacattttatgtattagttttgaaataataatggaaagtggctatgcagtttgaatatcctttgtttcagagccagatcatttcttggaaagtgtaggcttacctcaaataaatggctaactttatacatatttttaaagaaatatttatattgtatttatataatgtataaatggtttttataccaataaatggcattttaaaaaattc

SEQ ID NO: 103 - DNA, 5191 bp, Homo sapiens
gaattcagtaacccaggcattattttatcctcaagtcttaggttggttggagaaagataacaaaaagaaacatgattgtgcagaaacagacaaacctttttggaaagcatttgaaaatggcattccccctccacagtgtgttcacagtgtgggcaaattcactgctctgtcgtactttctgaaaatgaagaactgttacaccaaggtgaattatttataaattatgtacttgcccagaagcgaacagacttttactatcataagaacccttccttggtgtgctctttatctacagaatccaagacctttcaagaaaggtcttggattcttttcttcaggacactaggacataaagccacctttttatgatttgttgaaatttctcactccatcccttttgctgatgatcatgggtcctcagaggtcagacttggtgtccttggataaagagcatgaagcaacagtggctgaaccagagttggaacccagatgctctttccactaagcatacaactttccattagataacacctccctcccaccccaaccaagcagctccagtgcaccactttctggagcataaacataccttaactttacaacttgagtggccttgaatactgttcctatctggaatgtgctgttctctttcatcttcctctattgaagccctcctattcctcaatgccttgctccaactgcctttggaagattctgctcttatgcctccactggaattaatgtcttagtaccacttgtctattctgctatatagtcagtccttacattgctttcttcttctgatagaccaaactctttaaggacaagtacctagtcttatctatttctagatcccccacattactcagaaagttactccataaatgtttgtggaactgatttctatgtgaagacatgtgccccttcactctgttaactagcattagaaaaacaaatcttttgaaaagttgtagtatgcccctaagagcagtaacagttcctagaaactctctaaaatgcttagaaaaagatttattttaaattacctccccaataaaatgattggctggcttatcttcaccatcatgatagcatctgtaattaactgaaaaaaaataattatgccattaaaagaaaatcatccatgatcttgttctaacacctgccactctagtactatatctgtcacatggtctatgataaagttatctagaaataaaaaagcatacaattgataattcaccaaattgtggagcttcagtattttaaatgtatattaaaattaaattattttaaagatcaaagaaaactttcgtcatactccgtatttgataaggaacaaataggaagtgtgatgactcaggtttgccctgaggggatgggccatcagttgcaaatcgtggaatttcctctgacataatgaaaagatgagggtgcataagttctctagtagggtgatgatataaaaagccaccggagcactccataaggcacaaactttcagagacagcagagcacacaagcttctaggacaagagccaggaagaaaccaccggaaggaaccattctcactgtgtgtaaacatgacttccaagctggccgtggctctcttggcagccttcctgatttctgcagctctgtgtgaaggtaagcacatctttctgacctacagcgttttcctatgtctaaatgtgatccttagatagcaaagctattcttgatgctttggtaacaaacatcctttttattcagaaacagaatataatcttagcagtcaattaatgttaaattgaagatttagaaaaaactatatataacacttaggaaatataaaggtttgatcaatatagatattctgcttttataatttataccaggtagcatgcatatatttaacgtaaataagtaatttatagtatgtcctattgagaaccacggttacctatattatgtattaatattgagttgagcaaggtaactcagacaattccactccttgtagtatttcattgacaagcctcagatttgtcattaattcctgtctggtttaaagataccctgattatagaccaggcatgtataacttatttatatatttctgttaattctttctgaaggcaatttctatgctggagagtcttagcttgcctactataaataacactgtggtatcacagaggattatgcaatattgaccagataaaaataccatgaagatgttgatattgtacaaaaagaactctaactcttatataggaagttgttcaatgttgtcagttatgactgttttttaaaacaaagaactaactgaggtcaagggctaggagatattcaggaatgagttcactagaaacatgatgccttccatagtctccaaataatcatattggaattagaaggaagtagctggcagagctgtgcctgttgataaaatcaatccttaatcactttttcccccaacaggtgcagttttgccaaggagtgctaaagaacttagatgtcagtgcataaagacatactccaaacctttccaccccaaatttatcaaagaactgagagtgattgagagtggaccacactgcgccaacacagaaattatgtaagtactttaaaaaagattagatattttgttttagcaaacttaaaattaaggaaggtggaaatatttaggaaagttccaggtgttaggattacagtagtaaatgaaacaaaacaaaataaaaatatttgtctacatgacatttaaatatggtagcttccacaactactataaatgttattttggacttagactttatgcctgacttaaggaatcatgatttgaatgcaaaaactaaatattaatctgaaccatttctttcttatttcagtgtaaagctttctgatggaagagagctctgtctggaccccaaggaaaactgggtgcagagggttgtggagaagtttttgaagaggtaagttatatattttttaatttaaatttttcatttatcctgagacatataatccaaagtcagcctataaatttctttctgttgctaaaaatcgtcattaggtatctgcctttttggttaaaaaaaaaggaatagcatcaatagtgagtttgttgtacttatgaccagaaagaccatacatagtttgcccaggaaattctgggtttaagcttgtgtcctatactcttagtaaagttctttgtcactcccagtagtgtcctattttagatgataatttctttgatctccctatttatagttgagaatatagagcatttctaacacatgaatgtcaaagactatattgacttttcaagaaccctactttccttcttattaaacatagctcatctttatatttttaattttattttagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagatatacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttagtgtttttatgtgctctccaaattttttttactgtttctgattgtatggaaatataaaagtaaatatgaaacatttaaaatataatttgttgtcaaagtaatcaagtgtttgtcttttttttagttttagcttattgggattctctttgtttatatttaaaattatactttgatttagaaaacataaatgcttccccttagcattttgttatggaaaattacaaacttttatttttagaaaacagaactcctttccagaaataggttacaaacagtagtgtcctccacagaatgttggaaatgttttcaactccccactgtatactatcttgctaataagtctgtcttcagatttcgattaaccggtttgtatgtctgtgcactttagcatagctggacattaaagaggaaagagagtacatattataagttgcttatcagtaactgaggagtaaaactgataaatgtgaggcaaagaagtttaaaatatggttaaagcctaagcatatttgcaaacaaatcaaacaatactctgagaagtaaaaacataattatttaattaacaaatttcagtggataaattttataacaaattagacacagttgaaaataaaattagaaaactagaaaatagaacaaaagaaacttctggaattca

SEQ ID NO: 104 - DNA, 1572 bp, Homo sapiens
aatttctcactgcccctgtgataaactgtggtcactggctgtggcagcaactattataagatgctctgaaaactcttcagacactgaggggcaccagaggagcagactacaagaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaatgtaacccaacaagagcacattttatcatagcagagacatctgtatgcattcctgtcattacccattgtaacagagccacaaactaatactatgcaatgttttaccaataatgcaatacaaaagacctcaaaatacctgtgcatttcttgtaggaaaacaacaaaaggtaattatgtgtaattatactagaagttttgtaatctgtatcttatcattggaataaaatgacattcaataaataaaaa

SEQ ID NO: 105 - DNA, 1539 bp, Homo sapiens
ggaattccgggcccggtctttcctcccgccgccgccggcctggtcccggggactggcctccacgtccgactcgtccgagctgaagcccagcagcactttgctgccagccgcgggggcggcggaggcgcccccgggccctcccaggaggctctctgggccagaggccgagattcggcacaggcccccaggagtccgtaagtaggagaggtcgcccgagaccggccggacccccatccccgcggccgccgccgccgctggtcccgcggctgcgaccgtggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattgatcagttgaagctcattttaagactcgttggaaccccaggggctgagcttttgaagaaaatctcctcagagtctgcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgt

SEQ ID NO: 106 - DNA, 1155 bp, Homo sapiens
atgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtga

SEQ ID NO: 107 - DNA, 2000 bp, Homo sapiens
tttgggctgtgtgtgcgacgcgggtcggaggggcagtcgggggaaccgcgaagaagccgaggagcccggagccccgcgtgacgctcctctctcagtccaaaagcggcttttggttcggcgcagagagacccgggggtctagcttttcctcgaaaagcgccgccctgcccttggccccgagaacagacaaagagcaccgcagggccgatcacgctgggggcgctgaggccggccatggtcatggaagtgggcaccctggacgctggaggcctgcgggcgctgctgggggagcgagcggcgcaatgcctgctgctggactgccgctccttcttcgctttcaacgccggccacatcgccggctctgtcaacgtgcgcttcagcaccatcgtgcggcgccgggccaagggcgccatgggcctggagcacatcgtgcccaacgccgagctccgcggccgcctgctggccggcgcctaccacgccgtggtgttgctggacgagcgcagcgccgccctggacggcgccaagcgcgacggcaccctggccctggcggccggcgcgctctgccgcgaggcgcgcgccgcgcaagtcttcttcctcaaaggaggatacgaagcgttttcggcttcctgcccggagctgtgcagcaaacagtcgacccccatggggctcagccttcccctgagtactagcgtccctgacagcgcggaatctgggtgcagttcctgcagtaccccactctacgatcagggtggcccggtggaaatcctgccctttctgtacctgggcagtgcgtatcacgcttcccgcaaggacatgctggatgccttgggcataactgccttgatcaacgtctcagccaattgtcccaaccattttgagggtcactaccagtacaagagcatccctgtggaggacaaccacaaggcagacatcagctcctggttcaacgaggccattgacttcatagactccatcaagaatgctggaggaagggtgtttgtccactgccaggcaggcatttcccggtcagccaccatctgccttgcttaccttatgaggactaatcgagtcaagctggacgaggcctttgagtttgtgaagcagaggcgaagcatcatctctcccaacttcagcttcatgggccagctgctgcagtttgagtcccaggtgctggctccgcactgttcggcagaggctgggagccccgccatggctgtgctcgaccgaggcacctccaccaccaccgtgttcaacttccccgtctccatccctgtccactccacgaacagtgcgctgagctaccttcagagccccattacgacctctcccagctgctgaaaggccacgggaggtgaggctcttcacatcccattgggactccatgctccttgagaggagaaatgcaataactctgggaggggctcgagagggctggtccttatttatttaacttcacccgagttcctctgggtttctaagcagttatggtgatgacttagcgtcaagacatttgctgaactcagcacattcgggaccaatatatagtgggtacatcaagtccatctgacaaaatggggcagaagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggtttttttttagttgccaacagttgtatgtttgctgattatttatgacctgaaataatatatttcttcttctaagaagacattttgttacataaggatgacttttttatacaatggaataaattatggcatttctattg

SEQ ID NO: 108 - DNA, 5749 bp, Homo sapiens
cgcgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagggatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagcctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttagtgcaaggggaaacctgtgaaagctgcactcagagggaggagtctttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgagaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaaagttcatataatttaaagaaacactaaattagtttaaaatgaagcaatttatatctttatgcaaaaacatatgtctgtctttgcaaaggactgtaagcagattacaataaatcctttacttt

SEQ ID NO: 109 - DNA, 2062 bp, Homo sapiens
gtcagtccctcctgtagccgccgccgccgccgcccgccgcccctctgccagcagctccggcgccacctcgggccggcgtctccggcgggcgggagccaggcgctgacgggcgcggcgggggcggccgagcgctcctgcggctgcgactcaggctccggcgtctgcgcttccccatggggctggcctgcggcgcctgggcgctctgagattgtcactgctgttccaagggcacacgcagagggatttggaattcctggagagttgcctttgtgagaagctggaaatatttctttcaattccatctcttagttttccataggaacatcaagaaatcatgaacaactttggtaatgaagagtttgactgccacttcctcgatgaaggttttactgccaaggacattctggaccagaaaattaatgaagtttcttcttctgatgataaggatgccttctatgtggcagacctgggagacattctaaagaaacatctgaggtggttaaaagctctccctcgtgtcacccccttttatgcagtcaaatgtaatgatagcaaagccatcgtgaagacccttgctgctaccgggacaggatttgactgtgctagcaagactgaaatacagttggtgcagagtctgggggtgcctccagagaggattatctatgcaaatccttgtaaacaagtatctcaaattaagtatgctgctaataatggagtccagatgatgacttttgatagtgaagttgagttgatgaaagttgccagagcacatcccaaagcaaagttggttttgcggattgccactgatgattccaaagcagtctgtcgtctcagtgtgaaattcggtgccacgctcagaaccagcaggctccttttggaacgggcgaaagagctaaatatcgatgttgttggtgtcagcttccatgtaggaagcggctgtaccgatcctgagaccttcgtgcaggcaatctctgatgcccgctgtgtttttgacatgggggctgaggttggtttcagcatgtatctgcttgatattggcggtggctttcctggatctgaggatgtgaaacttaaatttgaagagatcaccggcgtaatcaacccagcgttggacaaatactttccgtcagactctggagtgagaatcatagctgagcccggcagatactatgttgcatcagctttcacgcttgcagttaatatcattgccaagaaaattgtattaaaggaacagacgggctctgatgacgaagatgagtcgagtgagcagacctttatgtattatgtgaatgatggcgtctatggatcatttaattgcatactctatgaccacgcacatgtaaagccccttctgcaaaagagacctaaaccagatgagaagtattattcatccagcatatggggaccaacatgtgatggcctcgatcggattgttgagcgctgtgacctgcctgaaatgcatgtgggtgattggatgctctttgaaaacatgggcgcttacactgttgctgctgcctctacgttcaatggcttccagaggccgacgatctactatgtgatgtcagggcctgcgtggcaactcatgcagcaattccagaaccccgacttcccacccgaagtagaggaacaggatgccagcaccctgcctgtgtcttgtgcctgggagagtgggatgaaacgccacagagcagcctgtgcttcggctagtattaatgtgtagatagcactctggtagctgttaactgcaagtttagcttgaattaagggatttggggggaccatgtaacttaattactgctagttttgaaatgtctttgtaagagtagggtcgccatgatgcagccatatggaagactaggatatgggtcacacttatctgtgttcctatggaaactatttgaatatttgttttatatggatttttattcactcttcagacacgctactcaagagtgcccctcagctgctgaacaagcatttgtagcttgtacaatggcagaatgggccaaaagcttagtgttgtgacctgtttttaaaataaagtatcttgaaataattaggc

SEQ ID NO: 110 - DNA, 3155 bp, Homo sapiens
gtttcattttgcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgtatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccaacctgcccagctgctgccgctggaggatccggaaagagtttccgaagagtgaagggcagtacagtggcttcaagagtccttactgaagccaggcaccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtcccccaaaaaaaaaaaagagtccttaccaatagcaggggctgcagtagccatgttaacatgacatttaccagcaacttgaacttcacctgcaaagctctgtggccacattttcagccaaagggaaatatgctttcatcttctgttgctctctgtgtctgagagcaaagtgacctggttaaacaaaccagaatccctctacatggactcagagaaaagagattgagatgtaagtctcaactctgtccccaggaagttgtgtgaccctaggcctctcacctctgtgcctctgtctccttgttgcccaactactatctcagagatattgtgaggacaaattgagacagtgcacatgaactgtcttttaatgtgtaaagatctacatgaatgcaaaacatttcattatgaggtcagactaggataatgtccaactaaaaacaaacccttttcatcctggctggagaatgtggagaactaaaggtggccacaaattctttgacactcaagtcccccaagacctaagggttttatctcctccccttgaatatgggtggctctgattgctttatccaaaagtggaagtgacattgtgtcagtttcagatcctgatcttaagaggctgacagcttctacttgctgtcccttggaactcttgctatcggggaagccagacgccatttaaaagtctgcctatcctggccaggtgtggtggctcacacctgtaatcccagcactttgggagaccaaggcgggcggatcacttaaagtcaggagtccaagaccagactcgccaacatggtgaaaccgtatctctaataaaaatacaaaaattagctgggcatggtgcgggcacctgtagtcctagctatcaagaggctgagacaggagaaacacttgaacctgggaggtggaggttgcattgagctgagatcgtgccactgcactccaggctgggtgacagagcgagactccatctcaaaaaaaaaaaaaagaaaaa

SEQ ID NO: 111 - DNA, 871 bp, Homo sapiens
ctgccaggcagtgcccttcccggagcgtgccctcgccgctgagctcccctgaacagcagctgcagcagccatggccccgccctgggtgcccgccatgggcttcacgctggcgcccagcctggggtgcttcgtgggctcccgctttgtccacggcgagggtctccgctggtacgccggcctgcagaagccctcgtggcacccgccccactgggtgctgggccctgtctggggcacgctctactcagccatggggtacggctcctacctggtctggaaagagctgggaggcttcacagagaaggctgtggttcccctgggcctctacactgggcagctggccctgaactgggcatggccccccatcttctttggtgcccgacaaatgggctgggccttggtggatctcctgctggtcagtggggcggcggcagccactaccgtggcctggtaccaggtgagcccgctggccgcccgcctgctctacccctacctggcctggctggccttcgcgaccacactcaactactgcgtatggcgggacaaccatggctggcatgggggacggcggctgccagagtgagtgcccggcccaccagggactgcagctgcaccagcaggtgccatcacgcttgtgatgtggtggccgtcacgctttcatgaccactgggcctgctagtctgtcagggccttggcccaggggtcagcagagcttcagaggttgccccacctgagcccccacccgggagcagtgtcctgtgctttctgcatgcttagagcatgttcttggaacatggaattttataagctgaataaagtttttgacttcctttaaaaaaaaaaaaaaaaaaaaaaaaaaaaaaaaaaaaaaaaa

SEQ ID NO: 112 - DNA, 2133 bp, Homo sapiens
atgctgtccttccagtaccccgacgtgtaccgcgacgagaccgccatacaggattatcatggtcataaaatttgtgacccttacgcctggcttgaagaccccgacagtgaacagactaaggcctttgtggaggcccagaataagattactgtgccatttcttgagcagtgtcccatcagaggtttatacaaagagagaatgactgaactatatgattatcccaagtatagttgccacttcaagaaaggaaaacggtatttttatttttacaatacaggtttgcagaaccagcgagtattatatgtacaggattccttagagggtgaggccagagtgttcctggaccccaacatactgtctgacgatggcacagtggcactccgaggttatgcgttcagcgaagatggtgaatattttgcctatggtctgagtgccagtggctcagactgggtgacaatcaagttcatgaaagttgatggtgccaaagagcttccagatgtgcttgaaagagtcaagttcagctgtatggcctggacccatgatgggaagggaatgttctacaactcataccctcaacaggatggaaaaagtgatggcacagagacatctaccaatctccaccaaaagctctactaccatgtcttgggaaccgatcagtcagaagatattttgtgtgctgagtttcctgatgaacctaaatggatgggtggagctgagttatctgatgatggctgctatgtcttgttatcaataagggaaggatgtgatccagtaaaccgactctggtactgtgacctacagcaggaatccagtggcatcgcgggaatcctgaagtgggtaaaactgattgacaactttgaaggggaatatgactacgtgaccaatgagggggcggtgttcacattcaagacgaatcgccagtctcccaactatcgcgtgatcaacattgacttcagggatcctgaagagtctaagtggaaagtacttgttcctgagcatgagaaagatgtcttagaatggatagcttgtgtcaggtccaacttcttggtcttatgctacctccatgacgtcaagaacattctgcagctccatgacctgactactggtgctctccttaagaccttcccgctcgatgtcggcagcattgtagggtacagcggtcagaagaaggacactgaaatcttctatcagtttacttcctttttatctccaggtatcatttatcactgtgatctgaccaaagaggagctggagccaagagtcttccgcgaggtgaccgtgaaaggaattgatgcttctgactaccagacagtccagcttttctaccctagcaaggatggtacgaagattccaatgttcattgtgcataaaaaaagcataaaattggatggctctcatccagctttcttatatggctatggcggcttcaacatatccatcacacccaactacagtgtttccaggcttatttttgtgagacacatgggtggtatcctggcagtggccaacatcagaggaggtggcgaatatggagagacgtggcataaaggtggtatcttggccaacaaacaaaactgctttgatgactttcagtgtgctgctgagtatctgatcaaggaaggttacacatctcccaagaggctgactattaatggaggttcaaatggaggcctcttagtggctgcttgtgcaaatcagagacctgacctctttggttgtgttattgcccaagttggagtaatggacatgctgaagtttcataaatataccatcggccatgcttggaccactgattatgggtgctcggacagcaaacaacactttgaatggcttgtcaaatactctccattgcataatgtgaagttaccagaagcagatgacatccagtacccgtccatgctgctcctcactgctgaccatgatgaccgcgtggtcccgcttcactccctgaagttcattgccacccttcagtacatcgtgggccgcagcaggaagcaaagcaaccccctgcttatccacgtggacaccaaggcgggccacggggcggggaagcccacagccaaagtgatagaggaagtctcagacatgtttgcgttcatcgcgcggtgcctgaacatcgactggattccgtaa

SEQ ID NO: 113 - DNA, 1375 bp, Homo sapiens
gcaaacagccggggctccagcgggagaacgataatgcaaagtgctatgttcttggctgttcaacacgactgcagacccatggacaagagcgcaggcagtggccacaagagcgaggagaagcgagaaaagatgaaacggacccttttaaaagattggaagacccgtttgagctacttcttacaaaattcctctactcctgggaagcccaaaaccggcaaaaaaagcaaacagcaagctttcatcaagccttctcctgaggaagcacagctgtggtcagaagcatttgacgagctgctagccagcaaatatggtcttgctgcattcagggcttttttaaagtcggaattctgtgaagaaaatattgaattctggctggcctgtgaagacttcaaaaaaaccaaatcaccccaaaagctgtcctcaaaagcaaggaaaatatatactgacttcatagaaaaggaagctccaaaagagataaacatagattttcaaaccaaaactctgattgcccagaatatacaagaagctacaagtggctgctttacaactgcccagaaaagggtatacagcttgatggagaacaactcttatcctcgtttcttggagtcagaattctaccaggacttgtgtaaaaagccacaaatcaccacagagcctcatgctacatgaaatgtaaaagggagcccagaaatggaggacatttcattctttttcctgaggggaaggactgtgacctgccataaagactgaccttgaattcagcctgggtgttcaggaaacatcactcagaactattgattcaaagttgggtagtgaatcaggaagccagtaactgactaggagaagctggtatcagaacagcttccctcactgtgtacagaacgcaagaagggaataggtggtctgaacgtggtgtctcactctgaaaagcaggaatgtaagatgatgaaagagacaatgtaatactgttggtccaaaagcatttaaaatcaatagatctgggattatgtggccttaggtagctggttgtacatctttccctaaatcgatccatgttaccacatagtagttttagtttaggattcagtaacagtgaagtgtttactatgtgcaacggtattgaagttcttatgaccacagatcatcagtactgttgtctcatgtaatgctaaaactgaaatggtccgtgtttgcattgttaaaaatgatgtgtgaaatagaatgagtgctatggtgttgaaaactgcagtgtccgttatgagtgccaaaaatctgtcttgaaggcagctacactttgaagtggtctttgaatacttttaataaatttattttgataaataatattgaacaaaaaaaaaaaaaaaaaaaaaaaaaa

SEQ ID NO: 114 - DNA, 1069 bp, Homo sapiens
aagtaattcctagacccgtaggtggccgcagagccggttacctctggttctgcgccagcgtgccccacccgcaggacggccgggttctttgatttgtacactttctaaaaccaaacccgagaggaagggcaggctcagggtggggatgccctgaaatattcgagagcaggaccgtttctactgaagagaagtttacaagaacgctctgtctggggcgggcgaggcctctgcgaggcgggtccgggagcgagggcagggcgtgggccgcgcgcccggggtcgggggagtcgggggcaggaagagggggaggagacagggctgggggagcgccctgccgagcgcccgccaggctcctcccgctcccgcgccgcctccctctacccacccgccgcacgtactaaggaaggcgcacagcccgccgcgctcgcctctccgccccgcgtccagctcgcccagctcgcccagcgtccgccgcgcctcggccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagtacacatgaagcagaagggaaagaagtaggcagaaatgagcagttcgctcctccctgataagagttgtcccaaagggtcgcttaaggaatctgccccacagcttcccccatagaaggatttcatgagcagatcaggacacttagcaaatgtaaaaataaaatctaactctcatttgacaagcagagaaagaaaagttaaataccagataagcttttgatttttgtattgtttgcatccccttgccctcaataaataaagttcttttttagttccaaatttgaaaaaaaaaaaaaaa

SEQ ID NO: 115 - DNA, 4535 bp, Homo sapiens
ggtccctcccctcctggctctggggtcgggcgcgcaccccgccccgtagcgcggcccctccctggcgagcgcaaccccatccagcgggagcgcggagccgcggccgcggggaagcattaagtttattcgcctcaaagtgacgcaaaaattcttcaagagctctttggcggcggctatctagagatcagaccatgtgagggcccgcgggtacaaatacggccgcgccggcgcccctccgcacagccagcgccgccgggtgcctcgagggcgcgaggccagcccgcctgcccagcccgggaccagcctccccgcgcagcctggcaggtctcctggaggcaaggcgaccttgcttgccctctcttgcagaataacaaggggcttagccacaggagttgctggcaagtggaaagaagaacaaatgagtcaatcccgacgtgtcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatataattttattgctggaatctactagaaccttctaatccatgtgctgctgtggcatcaggaaaggaagatgtaagaagctaaaatgaaaaatagtgtgtccatgcaagcttgtgagtctgtgtatattgttgtttcagtgtattcttatctctagtccaatattttgggcccattacaaatatatgaattccccaaatttttcttacattaacaaattctaccaactcaattgtgtatggaggttattatttgaagggtacaatcactacaacatgctctgccacccactccttttccagtgacactacttgagccacacactttcctttacaggccagcctctggcgtttgctgcacctcattgccaccttcctgtctctctgtgctaaacattcaggacagtgttccacaggcagatctggcctatttcattagtcaccatggcttggctgtgaagtacgttgaaggtggatcttgtcacatgccccttcagtgttcacctggccctctggtttaagttctgtctgccttacgtgactgagtttgactgtccaggttgctttgctcggtgaagagaggagggtaaatcggattctcgtttagcactgggttatacagatctggcaccctaacctaaaccaaggcatcttcactccaagagcagttggagagtctgggttagccttacgtggacctcgccgctcgctggcggtcacgattgtgagccctccagataatttttaaggttgagtctaagtaaggctgcttgggaaatggtcagctaagtaaatcacctttcatttcacataaggcccttaatatagataagtaaatttggcctttggtgtctcgtgactctcagaggcgtaggtagaggagcaaattaatatttgcagcatgggaattccttatcagaattttgaggggaataaatcctcatcagagacaaaaggacttaatcatctggccacctatcacttcagttctctgtataaatgaaatttaattctaacaaccttataaaaagaaggtccagacagcagaggaaacatcctgtccaattctaggttttcctcccttggcctcctttccccagcattgtctaccctggcccacttcctgcattctccccatgccctgctatttctgattctttgcttctcctagcgagatactttccttatatgatagctgctgagaagtttcccagaactgctagaggaaaagaagtggggaatttaggaaatatccctcactgacctaactccattatcttcactctttccttcttcctgccacctcatgcccattctctttactgtctagcatgctgaaagaaggaagtgatctaaatgccagcgtgttcagtggtaaatattagttggtgcaaaagaaaaaccatgattacttttgcactaacctaatagctttgcaaattttaagaacttgctttatgaagatattcggatatggattctccccaccccacatacttagacattgttcaaatatactacttttaaaaaaacaccttttcaaacagaattagcgttttgccaagtctggtattaatggaattgtacaggagctttgaaagttttcaaactttattaaactaaaaaaaaaaaatcgaaaa

SEQ ID NO: 116 - DNA, 1800 bp, Homo sapiens
actgcgacccggagccgcccggactgacggagcccactgcggtgcgggcgttggcgcgggcacggaggacccgggcaggcatcgcaagcgaccccgagcggagccccggagccatggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactgagatgagatcctcaaaaatcatcaaagtgtttaattgtataaaacagtgtttccagtgacacaactcatccagaactgtcttagtcataccatccatccctggtgaaagagtaaaaccaaaggttattatttcctttccatgg

SEQ ID NO: 117 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 118 - DNA, 987 bp, Homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 119 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 120 - DNA, 2245 bp, Homo sapiens
acgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcccgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaa

SEQ ID NO: 121 - DNA, 1526 bp, Homo sapiens
ccggaagtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtgtaaaaaaaaaaaaaaaaa

SEQ ID NO: 122 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 123 - DNA, 2439 bp, Homo sapiens
gagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcttttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaa

SEQ ID NO: 124 - DNA, 2276 bp, Homo sapiens
gaacgtggtataaaaggggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagagtgttgatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactgggcatggtggctcactgtctgtaatgtattacctgaggcagaagaccacctgagggtaggagtcaagatcagcctgggcaacatagtgagacgctgtctctacaaaaaataattagcctggcctggtggtgcatgcctagtcctagctgatctggaggctgacgtgggaggattgcttgagcctagagtgagctattatcatgccactgtacagcctgggtgttcacagatcttgtgtctcaaaggtaggcagaggcaggaaaagcaaggagccagaattaagaggttgggtcagtctgcagtgagttcatgcatttagaggtgttcttcaagatgactaatgtcaaaaattgagacatctgttgcggttttttttttttttttttcccctggaatgcagtggcgtgatctcagctcactgcagcctccgcctcctgggttcaagtgattctagtgcctcagcctcctgagtagctgggataatgggcgtgtgccaccatgcccagctaatttttgtatttttagtatagatggggtttcatcattttgaccaggctggtctcaaactcttgacctcagctgatgcgcctgccttggcctcccaaactgctgagattacagatgtgagccaccgcaccctacctcattttctgtaacaaagctaagcttgaacactgttgatgttcttgagggaagcatattgggctttaggctgtaggtcaagtttatacatcttaattatggtggaattcctatgtagagtctaaaaagccaggtacttggtgctacagtcagtctccctgcagagggttaaggcgcagactacctgcagtgaggaggtactgcttgtagcatatagagcctctccctagctttggttatggaggctttgaggttttgcaaacctgaccaatttaagccataagatctggtcaaagggatacccttcccactaaggacttggtttctcaggaaattatatgtacagtgcttgctggcagttagatgtcaggacaatctaagctgagaaaaccccttctctgcccaccttaacagacctctagggttcttaacccagcaatcaagtttgcctatcctagaggtggcggatttgatcatttggtgtgttgggcaatttttgttttactgtctggttccttctgcgtgaattaccaccaccaccacttgtgcatctcagtcttgtgtgttgtctggttacgtattccctgggtgataccattcaatgtcttaatgtacttgtggctcagacctgagtgcaaggtggaaataaacatcaaacatcttttcatta

SEQ ID NO: 125 - DNA, 1229 bp, Homo sapiens
gtctgacgggcgatggcgcagccaatagacaggagcgctatccgcggtttctgattggctactttgttcgcattataaaaggcacgcgcgggcgcgaggcccttctctcgccaggcgtcctcgtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttctttaaaaagtaaaaaaaaaaaaaaaaaaaaaaaaa

SEQ ID NO: 126 - DNA, 1142 bp, Homo sapiens
cttttccaagcggctgccgaagatggcggaggtgcaggtcctggtgcttgatggtcgaggccatctcctgggccgcctggcggccatcgtggctaaacaggtactgctgggccggaaggtggtggtcgtacgctgtgaaggcatcaacatttctggcaatttctacagaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccaccgccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaaa

SEQ ID NO: 127 - DNA, 2405 bp, Homo sapiens
tccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaaaaaaaaaaaaaaaaa

SEQ ID NO: 128 - DNA, 1867 bp, Homo sapiens
ggttcgctgtggcgggcgcctgggccgccggctgtttaacttcgcttccgctggcccatagtgatctttgcagtgacccagcagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttaaaaaaaaaaaaaaaaaaaaaa

SEQ ID NO: 129 - DNA, 5241 bp, Homo sapiens
agagcgtcgggatatcgggtggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaaaaaaaaa

SEQ ID NO: 130 - DNA, 2602 bp, Homo sapiens
gagccgcggctaaggaacgcgggccgcccacccgctcccggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattccttctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaaaaaaaa

SEQ ID NO: 131 - DNA, 3003 bp, Homo sapiens
ctttctccttccccttcttccgggctcccgtcccggctcatcacccggcctgtggcccactcccaccgccagctggaaccctggggactacgacgtccctcaaaccttgcttctaggagataaaaagaacatccagtcatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggatacccaaggagacgaagctgaagcaggagaaggaggggaaaattaaccggccttccaacttttgtctgcctcattctaaaatttacacagtagaccatttgtcatccatgctgtcccacaaatagttttttgtttacgatttatgacaggtttatgttacttctatttgaatttctatatttcccatgtggtttttatgtttaatattaggggagtagagccagttaacatttagggagttatctgttttcatcttgaggtggccaatatggggatgtggaatttttatacaagttataagtgtttggcatagtacttttggtacattgtggcttcaaaagggccagtgtaaaactgcttccatgtctaagcaaagaaaactgcctacatactggtttgtcctggcggggaataaaagggatcattggttccagtcacaggtgtagtaattgtgggtactttaaggtttggagcacttacaaggctgtggtagaatcataccccatggataccacatattaaaccatgtatatctgtggaatactcaatgtgtacacctttgactacagctgcagaagtgttcctttagacaaagttgtgacccattttactctggataagggcagaaacggttcacattccattatttgtaaagttacctgctgttagctttcattatttttgctacactcattttatttgtatttaaatgttttaggcaacctaagaacaaatgtaaaagtaaagatgcaggaaaaatgaattgcttggtattcattacttcatgtatatcaagcacagcagtaaaacaaaaacccatgtatttaacttttttttaggatttttgcttttgtgatttttttttttttgatacttgcctaacatgcatgtgctgtaaaaatagttaacagggaaataacttgagatgatggctagctttgtttaatgtcttatgaaattttcatgaacaatccaagcataattgttaagaacacgtgtattaaattcatgtaagtggaataaaagttttatgaatggacttttcaactactttctctacagcttttcatgtaaattagtcttggttctgaaacttctctaaaggaaattgtacattttttgaaatttattccttattccctcttggcagctaatgggctcttaccaagtttaaacacaaaatttatcataacaaaaatactactaatataactactgtttccatgtcccatgatcccctctcttcctccccaccctgaaaaaaatgagttcctattttttctgggagagggggggattgattagaaaaaaatgtagtgtgttccatttaaaattttggcatatggcattttctaacttaggaagccacaatgttcttggcccatcatgacattgggtagcattaactgtaagttttgtgcttccaaatcactttttggtttttaagaatttcttgatactcttatagcctgccttcaattttgatcctttattctttctatttgtcaggtgcacaagattaccttcctgttttagccttctgtcttgtcaccaaccattcttacttggtggccatgtacttggaaaaaggccgcatgatctttctggctccactcagtgtctaaggcaccctgcttcctttgcttgcatcccacagactatttccctcatcctatttactgcagcaaatctctccttagttgatgagactgtgtttatctccctttaaaaccctacctatcctgaatggtctgtcattgtctgcctttaaaatccttcctctttcttcctcctctattctctaaataatgatggggctaagttatacccaaagctcactttacaaaatatttcctcagtactttgcagaaaacaccaaacaaaaatgccattttaaaaaaggtgtattttttcttttagaatgtaagctcctcaagagcagggacaatgttttctgtatgttctattgtgcctagtacactgtaaatgctcaataaatattgatgatgggaggcagtgagtcttgatgataagggtgagaaactgaaatcccaaacactgttttgttgcttgttttattatgacctcagattaaattgggaaatattggcccttttgaataattgtcccaaatattacattcaaataaaagtgcaatggagaaaaaaaaaaa

SEQ ID NO: 132 - DNA, 1869 bp, Homo sapiens
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

(19) United States
(12) Patent Application Publication
(10) Pub. No.: US 2011/0172501 A1
(43) Pub. Date: Jul. 14, 2011

(54) SYSTEM AND METHODS FOR MEASURING BIOMARKER PROFILES

(76) Inventors: Irina Antonijevic, Raritan, NJ (US); Joseph Tamm, Hawthorne, NJ (US); Roman Artymyshyn, Flemington, NJ (US); Christophe P.G. Gerald, Mahwah, NJ (US); Jan Bastholm Vistisen, Aars (DK)

(21) Appl. No.: 13/000,405

(22) PCT Filed: Aug. 27, 2009
(86) PCT No.: PCT/US2009/055144

§ 371 Date: Mar. 18, 2011

Related U.S. Application Data
(60) Provisional application No. 61/092,270, filed on Aug. 27, 2008.

(51) Int. Cl. A61B 5/00

(52) U.S. Cl. 600/300

(57) ABSTRACT

The present invention relates to methods and systems for diagnosing patients with affective disorders. The methods are also useful for predicting the susceptibility for an affective disorder in a subject.

This application contains a Sequence Listing, submitted in electronic form as filename 71021-WO-PCT_SequenceListing_ST25.txt, of size 148,658 bytes, created on Aug. 25, 2009. The sequence listing is hereby incorporated by reference in its entirety.

### 1 FIELD OF THE INVENTION

The present invention provides methods and compositions of identifying transcription profiles in a subject suffering from a disorder by profiling and comparing mRNA expression levels of genes in control subjects relative to that of diseased subjects. The present invention further provides methods and compositions for predicting and diagnosing disorders, such as affective disorders, in a subject by determining a transcription profile related to biomarkers in such subject.

### 2 BACKGROUND OF THE INVENTION

Throughout this application various publications are referred to by citations within parenthesis. The disclosures of these publications, in their entireties, are hereby incorporated by reference into this application in order to more fully describe the state of the art to which the invention pertains.

Current psychiatric diagnostic classifications, particularly those for affective disorders, lack a distinct clinical description, and include no biological features to delineate one diagnostic entity from another. Although today's classifications allow to further specify the clinical features of affective disorders, e.g. major depressive disorder, the criteria remain a matter of significant debate and do not necessarily follow a biological rationale (Parker, et al. Am. J. Psychiatry 2000, 157(8): 1195-1203).

Among affective disorders, many clinical segments exist, such as bipolar disorders I and II, dysthymia, and major depressive disorders, including psychotic depression, severe vs. mild or moderate depression, melancholic vs. atypical depression, etc. As such, no distinct biological markers or biomarkers have been described for these segments. Moreover, lack of segmentation for specific disorders can have treatment implications. Furthermore, comorbidity is problematic for physicians who cannot delineate the presence of two disorders.

Altogether, the clinical assessments in psychiatry and the non-specific clinical diagnostic criteria highlight the need for biological markers in order to recognize patients that share a similar biology. This seems a particular dilemma for affective disorders, as there is emerging evidence for the existence of subtypes that show clinical differences and distinct biological features (Gold and Chrousos, Mol. Psychiatry 2002, 7(3): 254-275). So far, however, no biological markers have been consistently shown to delineate a segment of the patient population with respect to affective disorders.

Previous studies have explored tests that measure biological changes in subjects with depression vs. control subjects, or subjects before and after treatment, such as the dexamethasone/corticotrophin releasing hormone (DEX/CRH) test. However, such tests have been examined in small numbers of patients, have not been reproduced, and/or have not linked a biological read-out with a specific phenotype. (Ising, M. et al., Biol. Psychiatry, 2006 Nov. 20, e-pub ahead of print; Kunugi, H. et al., Neuropsychopharm. 2006, 31(1): 212-20). This is pertinent as clinically relevant biomarkers must be associated with a specific biology and a specific phenotype, and ideally, should be returned to normal levels by treatment.

Protein biomarkers have been identified for diabetes, Alzheimer's Disease, and cancer. (See, for Example, U.S. Pat. Nos. 7,125,663; 7,097,989; 7,074,576; and 6,925,389.) However, methods for detection of protein biomarkers, such as mass spectrometry and specific binding to antibodies, often yield irreproducible data, and these methods are not favorable to high throughput use.

High throughput expression analysis methods using microarrays, have been used to assess gene expression changes with mixed results or no relevant outcome (Brenner, S. et al Nat Biotechnol. 2000, 18(6):597-8; Schena et al. Science. 1995, 270(5235):467-70; Velculescu, V. E. et al, Science. 1995, 270(5235):484-7). Due to the large ratio of measured gene expressions to the number of subjects, and given the heterogeneity of depressive disorders, a large number of false positives are to be expected with microarray data. (See, for review, Iwamoto K, and Kato T., Neuroscientist 2006, 12(4):349-61; Bunney W E, et al., Am J Psychiatry 2003, 160(4):657-66; and Iga J, Ueno S, and Ohmori T., Ann Med 2008, 40(5):336-42.) Sibille et al. (Neuropsychopharm. 2004, 29(2):351-61) performed a large scale genomic analysis, however found no evidence for molecular differences that correlated with depression and suicide, and could not reproduce changes in expression levels for genes that were previously found to be associated with depression. Because of such difficulties, consistent profiles have not been identified.

Focused arrays and qPCR for multiple relevant genes have been used for identifying stress related genes, but these studies have not yet identified a diagnostic profile related to depression (Rokutan et al, J. Med. Invest. 2005, 52(3-4):137-44; Ohmori et al., J. Med. Invest. 2005, 52 (Suppl):266-71). In rat brain regions, transcriptional changes of particular genes have been implicated in the control of mood and anxiety, however these changes are not correlated to human blood samples (WO2007106685A2).

### 3 SUMMARY OF THE INVENTION

The present invention provides a method of diagnosing an affective disorder in a test subject, the method comprising: evaluating whether a plurality of features of a plurality of biomarkers in a biomarker profile of the test subject satisfies a value set, wherein satisfying the value set predicts that the test subject has said affective disorder, and wherein the plurality of features are measurable aspects of the plurality of biomarkers, the plurality of biomarkers comprising at least two biomarkers listed in Table 1A.

The present invention also provides a computer program product, wherein the computer program product comprises a computer readable storage medium and a computer program mechanism embedded therein, the computer program mechanism comprising instructions for carrying out the diagnostic method.

One aspect of the invention provides a computer comprising one or more processors and a memory coupled to the one or more processors, the memory storing instructions for carrying out the diagnostic method.

Another aspect of the invention provides a method of determining a likelihood that a test subject exhibits a symptom of an affective disorder, the method comprising: evaluating whether a plurality of features of a plurality of biomarkers in a biomarker profile of the test subject satisfies a value set, wherein satisfying the value set provides said likelihood that the test subject exhibits a symptom of an affective disorder, and wherein the plurality of features are measurable aspects of the plurality of biomarkers, the plurality of biomarkers comprising at least two biomarkers listed in Table 1A.

The present invention provides, in another aspect, a transcription profile which is a measure of transcriptional analysis for each biological sample collected from a plurality of control subjects. For example, the present invention provides a transcription profile which is a measure of transcriptional analysis for each biological sample collected from a plurality of depressed, severely depressed, or bipolar subjects. The present invention further provides a transcription profile which is a measure of transcriptional analysis for each biological sample collected from a plurality of borderline personality disorder subjects. The present invention also provides a transcription profile which is a measure of transcriptional analysis for each biological sample collected from a plurality of PTSD subjects.

The invention also provides that a transcription profile comprising the collective measure of a first plurality of control subjects is stored, for example in a database. A transcription profile comprising the collective measure of a second plurality of subjects, for example, diseased subjects, is compared to the transcription profile of the first plurality of control subjects using a classification algorithm. The classification algorithm provides output that classifies each of the subjects.

The present invention provides a method for diagnosing an affective disorder by identifying a transcription profile in a patient, comparing such transcription profile to the profile of a control subject or group of control subjects, thereby diagnosing the patient's affective disorder based on the presence or absence of changes in the transcription profile.

One aspect of the invention provides a method for diagnosing a subject with an affective disorder comprising:
(a) obtaining biological samples from a plurality of control subjects and from a plurality of diseased subjects;
(b) measuring the mRNA expression level of genes in the samples of the plurality of control subjects and the plurality of diseased subjects, wherein the genes are selected from the group consisting of ADA, ARRB1, ARRB2, CD8a, CD8b, CREB1, CREB2, DPP4, ERK1, ERK2, Gi2, Gs, GR, IL 1b, IL6, IL8, INDO, MAPK14, MAPK8, MKP1, MR, ODC1, P2X7, PBR, PREP, RGS2, S100A10, SERT and VMAT2;
(c) collecting and storing the mRNA expression levels for each gene from the plurality of control subjects and the plurality of diseased subjects as mRNA data in a computer medium;
(d) processing such mRNA data by means of a classification algorithm; and
(e) providing output data which classifies the subject,
thereby diagnosing the subject with an affective disorder.

The present invention further provides methods for predicting a subject's susceptibility to an affective disorder by comparing the subject's transcription profile of genes selected from the group consisting of ADA, ARRB1, ARRB2, CD8a, CD8b, CREB1, CREB2, DPP4, ERK1, ERK2, Gi2, Gs, GR, IL1b, IL6, IL8, INDO, MAPK14, MAPK8, MKP1, MR, ODC1, P2X7, PBR, PREP, RGS2, S100A10, SERT and VMAT2, to the transcription profile of genes of a plurality of control subjects.

One aspect of the invention provides a method for predicting the likelihood of a subject exhibiting symptoms of an affective disorder comprising:
(a) obtaining biological samples from a plurality of control subjects and from a plurality of diseased subjects;
(b) measuring the mRNA expression level of genes in the samples of the plurality of control subjects and the plurality of diseased subjects, wherein the genes are selected from the group consisting of ADA, ARRB1, ARRB2, CD8a, CD8b, CREB1, CREB2, DPP4, ERK1, ERK2, Gi2, Gs, OR, IL 1b, IL6, IL8, INDO, MAPK14, MAPK8, MKP1, MR, ODC1, P2X7, PBR, PREP, RGS2, S100A10, SERT and VMAT2;
(c) collecting and storing the mRNA expression levels for each gene from the plurality of control subjects and the plurality of diseased subjects as mRNA data in a computer medium;
(d) processing such mRNA data by means of a classification algorithm; and
(e) providing output data which classifies the subject,
thereby predicting the likelihood of a subject exhibiting symptoms of an affective disorder.

### 4 BRIEF DESCRIPTION OF THE FIGURES

FIG. 1 is an illustration of a computer system in accordance with an embodiment of the present invention.

FIGS. 2A and 2B. Scatterplots showing relative mRNA levels of ARRB1 (beta-arrestin 1) and Gi2 (guanine nucleotide binding protein alpha i2), respectively, in control subjects vs. depressed subjects, as measured by copies/ng cDNA by qPCR methods (p<0.001; Mann Whitney test).

FIGS. 3A and 3B. Scatterplots showing relative mRNA levels of MAPK14 (p38 mitogen-activated protein kinase 14) and ODC1 (ornithine decarboxylase 1), respectively, in control subjects vs. depressed subjects, as measured by copies/ng cDNA by qPCR methods (p<0.001; Mann Whitney test).

FIGS. 4A, 4B and 4C. Scatterplots showing relative mRNA levels of ERK1 (extracellular signal-regulated kinase 1), Gi2 (guanine nucleotide binding protein alpha i2), and MAPK14 (p38 mitogen-activated protein kinase 14), respectively, in control subjects vs. severely depressed subjects, as measured by copies/ng cDNA by qPCR methods (p<0.001; Mann Whitney test).

FIGS. 5A, 5B and 5C. Scatterplots showing relative mRNA levels of Gi2 (guanine nucleotide binding protein alpha i2), GR (alpha-glucocorticoid receptor), and MAPK14 (p38 mitogen-activated protein kinase 14), respectively, in control subjects vs. severely depressed/bipolar subjects, as measured by copies/ng cDNA by qPCR methods (p<0.001; Mann Whitney test).

FIGS. 6A, 6B and 6C. Scatterplots showing relative mRNA levels of Gi2 (guanine nucleotide binding protein alpha i2), MAPK14 (p38 mitogen-activated protein kinase 14), and MR (mineralocorticoid receptor), respectively, in control subjects vs. borderline personality disorder subjects, as measured by copies/ng cDNA by qPCR methods (p<0.001; Mann Whitney test).

FIGS. 7A, 7B and 7C. Scatterplots showing relative mRNA levels of ARRB2 (beta-arrestin 2), ERK2 (extracellular signal-regulated kinase 2), and RGS2 (regulator of G-protein signaling 2), respectively, in 196 control subjects vs. 66 acute PTSD subjects, as measured by copies/ng cDNA by qPCR methods (p<0.001; Mann Whitney test).

FIGS. 8A and 8B. FIG. 8A is an illustration of the performance of the SLR algorithm, which performs both the gene selection and training, scoring an accuracy of 93%, PPV=93%, and NPV=94% in the classification of depressed subjects vs. controls. The Support Vector Machine (SVM) classifier, preceded by RF gene selection, scores an accuracy of 88%, PPV=89% and NPV=88% in the classification of depressed subjects vs. controls. FIG. 8B shows Random Forest (RF) selecting 14 genes and Stepwise Logistic Regression (SLR) selecting 17 genes from Table 1A based on the statistical parameters of each method in the classification of depressed subjects vs. controls. The overlapping genes selected by both RF and SLR methods at the selection step of the classification process are shown in gray.

FIG. 9 depicts genes for which the mean expression levels (transcript values) were significantly different (p<0.05) between severely depressed patients and controls. These genes are ranked according to the magnitude of the calculated −Log(p) value, as seen in Table 5A.

FIG. 10 represents the distribution of severely depressed subjects and control subjects according to the transcription profile consisting of ERK1 and MAPK14 for each subject. Severely depressed subjects are represented by open circles (∘) and control subjects are represented by closed triangles (▴). The X and Y axis depict transcript values (copies/ng cDNA) for ERK1 and MAPK14, respectively.

FIG. 11 represents the distribution of severely depressed subjects and control subjects according to the transcription profile consisting of Gi2 and IL1b for each subject. Severely depressed subjects are represented by open circles (∘) and control subjects are represented by closed triangles (▴). The X and Y axis depict transcript values (copies/ng cDNA) for Gi2 and IL1b, respectively.

FIG. 12 represents the distribution of severely depressed subjects and control subjects according to the transcription profile consisting of ERK1 and IL1b for each subject. Severely depressed subjects are represented by open circles (∘) and control subjects are represented by closed triangles (▴). The X and Y axis depict transcript values (copies/ng cDNA) for ERK1 and IL 1b, respectively.

FIG. 13 represents the distribution of severely depressed subjects and control subjects according to the transcription profile consisting of ARRB1 and MAPK14 for each subject. Severely depressed subjects are represented by open circles (∘) and control subjects are represented by closed triangles (▴). The X and Y axis depict transcript values (copies/ng cDNA) for ARRB1 and MAPK14, respectively.

### 5 DETAILED DESCRIPTION OF THE INVENTION

The present invention allows for the rapid and accurate diagnosis of an affective disorder by evaluating biomarker features in biomarker profiles. These biomarker profiles are constructed from biological samples of subjects.

### 5.1 Definitions

As used herein, “affective disorder” shall mean a mental disorder characterized by a consistent, pervasive alteration of mood, and affecting thoughts, emotions and behaviors. Examples of affective disorders include, but are not limited to, depressive disorders, anxiety disorders, bipolar disorders, dysthymia and schizoaffective disorders. Anxiety disorders include, but are not limited to, generalized anxiety disorder, panic disorder, obsessive-compulsive disorder, phobias, and post-traumatic stress disorder. Depressive disorders include, but are not limited to, major depressive disorder (MDD), catatonic depression, melancholic depression, atypical depression, psychotic depression, postpartum depression, bipolar depression and mild, moderate or severe depression. Personality disorders include, but are not limited to, paranoid, antisocial and borderline personality disorders.

A “biomarker” is virtually any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a biological sample, or any other characteristic that is objectively measured and evaluated as an indicator of normal biologic processes, pathogenic processes, or pharmacologic responses to a therapeutic intervention, or an indication thereof. See Atkinson, A. J., et al. Biomarkers and Surrogate Endpoints: Preferred Definitions and Conceptual Framework, Clinical Pharm. & Therapeutics, 2001 March; 69(3): 89-95. “Derived from” as used in this context refers to a compound that, when detected, is indicative of a particular molecule being present in the biological sample. For example, detection of a particular cDNA can be indicative of the presence of a particular RNA transcript in the biological sample. As another example, detection of or binding to a particular antibody can be indicative of the presence of a particular antigen (e.g., protein) in the biological sample. Here, a discriminating molecule or fragment is a molecule or fragment that, when detected, indicates presence or abundance of an above-identified compound.

A biomarker can, for example, be isolated from the biological sample, directly measured in the biological sample, or detected in or determined to be in the biological sample. A biomarker can, for example, be functional, partially functional, or non-functional. In one embodiment, a biomarker is isolated and used, for example, to raise a specifically-binding antibody that can facilitate biomarker detection in a variety of diagnostic assays. Any immunoassay may use any antibodies, antibody fragment or derivative thereof capable of binding the biomarker molecules (e.g., Fab, F(ab′)₂, Fv, or scFv fragments). Such immunoassays are well-known in the art. In addition, if the biomarker is a protein or fragment thereof, it can be sequenced and its encoding gene can be cloned using well-established techniques.

As used herein, the term “a species of a biomarker” refers to any discriminating portion or discriminating fragment of a biomarker described herein, such as a splice variant of a particular gene described herein (e.g., a gene listed in Table 1A, infra). Here, a discriminating portion or discriminating fragment is a portion or fragment of a molecule that, when detected, indicates presence or abundance of the above-identified transcript, cDNA, amplified nucleic acid, or protein.

A “biomarker profile” comprises a plurality of one or more types of biomarkers (e.g., an mRNA molecule, a cDNA molecule, a protein and/or a carbohydrate, or an indication thereof, etc.), together with a feature, such as a measurable aspect (e.g., abundance) of the biomarkers. A biomarker profile comprises at least two such biomarkers, where the biomarkers can be in the same or different classes, such as, for example, a nucleic acid and a carbohydrate. A biomarker profile may also comprise at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 or more biomarkers. In one embodiment, a biomarker profile comprises hundreds, or even thousands, of biomarkers. A biomarker profile can further comprise one or more controls or internal standards. In one embodiment, the biomarker profile comprises at least one biomarker that serves as an internal standard. The term “indication” as used herein in this context merely refers to a situation where the biomarker profile contains symbols, data, abbreviations or other similar indicia for a nucleic acid, an mRNA molecule, a cDNA molecule, a protein and/or a carbohydrate, or any other form of biomarker, rather than the biomarker molecular entity itself. For instance, an exemplary biomarker profile of the present invention comprises the names of the genes in Table 1A.

Each biomarker in a biomarker profile includes a corresponding “feature.” A “feature”, as used herein, refers to a measurable aspect of a biomarker. A feature can include, for example, the presence or absence of biomarkers in the biological sample from the subject as illustrated in exemplary biomarker profile 1:

### Exemplary Biomarker Profile 1

| | Feature |
|---|---|
| Biomarker | Presence in sample |
| transcript of gene A | Present |
| transcript of gene B | Absent |

In exemplary biomarker profile 1, the feature value for the transcript of gene A is “presence” and the feature value for the transcript of gene B is “absence.”

A feature can include, for example, the abundance of a biomarker in the biological sample from a subject as illustrated in exemplary biomarker profile 2:

### Exemplary Biomarker Profile 2

| | Feature |
|---|---|
| | Abundance in sample |
| Biomarker | in relative units |
| transcript of gene A | 300 |
| transcript of gene B | 400 |

In exemplary biomarker profile 2, the feature value for the transcript of gene A is 300 units and the feature value for the transcript of gene B is 400 units.

A feature can also be a ratio of two or more measurable aspects of a biomarker as illustrated in exemplary biomarker profile 3:

### Exemplary Biomarker Profile 3

| | Feature |
|---|---|
| | Ratio of abundance of |
| | transcript of gene A/ |
| Biomarker | transcript of gene B |
| transcript of gene A | 300/400 |
| transcript of gene B | |

In exemplary biomarker profile 3, the feature value for the transcript of gene A and the feature value for the transcript of gene B is 0.75 (300/400).

In some embodiments, there is a one-to-one correspondence between features and biomarkers in a biomarker profile as illustrated in exemplary biomarker profile 1, above. In some embodiments, the relationship between features and biomarkers in a biomarker profile of the present invention is more complex, as illustrated in Exemplary biomarker profile 3, above.

Those of skill in the art will appreciate that other methods of computation of a feature can be devised and all such methods are within the scope of the present invention. For example, a feature can represent the average of an abundance of a biomarker across biological samples collected from a subject at two or more time points. Furthermore, a feature can be the difference or ratio of the abundance of two or more biomarkers from a biological sample obtained from a subject in a single time point. A biomarker profile may also comprise at least two, three, four, five, 10, 20, 30 or more features. In one embodiment, a biomarker profile comprises hundreds, or even thousands, of features.

In some embodiments, features of biomarkers are measured using quantitative PCR (qPCR). The use of qPCR to measure gene transcript abundance is well known. In some embodiments, features of biomarkers are measured using microarrays. The construction of microarrays and the techniques used to process microarrays in order to obtain abundance data is well known, and is described, for example, by Draghici, 2003, Data Analysis Tools for DNA Microarrays, Chapman & Hall/CRC, and international publication number WO 03/061564. A microarray comprises a plurality of probes. In some instances, each probe recognizes, e.g., binds to, a different biomarker. In some instances, two or more different probes on a microarray recognize, e.g., bind to, the same biomarker. Thus, typically, the relationship between probe spots on the microarray and a subject biomarker is a two to one correspondence, a three to one correspondence, or some other form of correspondence. However, it can be the case that there is a unique one-to-one correspondence between probes on a microarray and biomarkers.

As used herein, the term “complementary,” in the context of a nucleic acid sequence (e.g., a nucleotide sequence encoding a gene described herein), refers to the chemical affinity between specific nitrogenous bases as a result of their hydrogen bonding properties. For example, guanine (G) forms a hydrogen bond with only cytosine (C), while adenine forms a hydrogen bond only with thymine (T) in the case of DNA, and uracil (U) in the case of RNA. These reactions are described as base pairing, and the paired bases (G with C, or A with T/U) are said to be complementary. Thus, two nucleic acid sequences may be complementary if their nitrogenous bases are able to form hydrogen bonds. Such sequences are referred to as “complements” of each other. Such complement sequences can be naturally occurring, or, they can be chemically synthesized by any method known to those skilled in the art, as for example, in the case of antisense nucleic acid molecules which are complementary to the sense strand of a DNA molecule or an RNA molecule (e.g., an mRNA transcript). See, e.g., Lewin, 2002, Genes VII. Oxford University Press Inc., New York, N.Y.

As used herein, a “data analysis algorithm” is an algorithm used to construct a decision rule using biomarker profiles of subjects in a training population. Representative data analysis algorithms are described below. A “decision rule” is the final product of a data analysis algorithm, and is characterized by one or more value sets, where each of these value sets is indicative of an aspect of an affective disorder, the onset of an affective disorder, a prediction that a subject will an affective disorder, or a likelihood that a subject exhibits a symptom of an affective disorder. In one specific example, a value set represents a prediction that a subject will develop an affective disorder. In another example, a value set represents a prediction that a subject will not develop an affective disorder.

A “decision rule” is a method used to evaluate biomarker profiles. Such decision rules can take on one or more forms that are known in the art, as exemplified in Hastie et al., 2001, The Elements of Statistical Learning, Springer-Verlag, New York. A decision rule may be used to act on a data set of features to, inter alia, predict the presence of an affective disorder, or the likelihood that a subject exhibits or has a symptom of an affective disorder, or exhibits a susceptibility to developing an affective disorder. Exemplary decision rules that can be used in some embodiments of the present invention are described in further detail below.

As used herein, the term “endophenotype” shall mean a heritable characteristic, such as a biomarker, that is associated with illness, which characteristic is present whether or not the individual is symptomatic. (For review see Lenox et al., 2002, American Journal of Medical Genetics (Neuropsychiatric Genetics) 114:391-406)

As used herein, the terms “gene expression profile” and “transcription profile” are biomarker profiles determined by relative measurement of messenger ribonucleic acid (mRNA) levels of selected genes. Transcription profiles are measured by transcriptional analysis of genes from a biological sample of a subject or patient.

As used herein, “healthy control subjects,” “healthy controls,” and “control subjects” shall mean subjects that are free of major current medical or psychiatric problems, but may, e.g. suffer from headaches. Control subjects preferably have low body mass index (BMI, less than 30), no drug use for the past three months, and low or zero stress scores, family history scores, and symptom scores. Control subjects may be free from any history of psychiatric diseases, any history of substance abuse, any family history of psychiatric diseases, any early life stressors or any recent stressors, as determined by a self-administered questionnaire. Control subjects can, but need not be further evaluated by a physician prior to obtaining biological samples.

The terms “obtain” and “obtaining,” as used herein, mean “to come into possession of,” or “coming into possession of,” respectively. This can be done, for example, by retrieving data from a data store in a computer system. This can also be done, for example, by direct measurement.

As used herein, the term “phenotype” shall mean measurable and/or observable biological, clinical or behavioral characteristics that are the result of a subject's genotype and the environment.

As used herein, the terms “protein”, “peptide”, and “polypeptide” are, unless otherwise indicated, interchangeable.

As used herein, “PTSD control subjects” shall mean subjects that have not been subjected to an extreme traumatic stressor and have been assessed by a physician to be free of any neuropsychiatric disease. The PTSD control subjects of this invention are generally matched subjects, for example, from the same geographical region and of the same gender as the subjects exhibiting the disorder.

As used herein, the term “specifically,” and analogous terms, in the context of an antibody, refers to peptides, polypeptides, and antibodies or fragments thereof that specifically bind to an antigen or a fragment and do not specifically bind to other antigens or other fragments. A peptide or polypeptide that specifically binds to an antigen may bind to other peptides or polypeptides with lower affinity, as determined by standard experimental techniques, for example, by any immunoassay well-known to those skilled in the art. Such immunoassays include, but are not limited to, radioimmunoassays (RIAs) and enzyme-linked immunosorbent assays (ELISAs). Antibodies or fragments that specifically bind to an antigen may be cross-reactive with related antigens. Preferably, antibodies or fragments thereof that specifically bind to an antigen do not cross-react with other antigens. See, e.g., Paul, ed., 2003, Fundamental Immunology, 5th ed., Raven Press, New York at pages 69-105, for a discussion regarding antigen-antibody interactions, specificity and cross-reactivity, and methods for determining all of the above.

As used herein, a “subject” is an animal, preferably a mammal, more preferably a non-human primate, and most preferably a human. The terms “subject,” “individual,” “candidate,” and “patient” are used interchangeably herein. In some embodiments, the subject is an animal. In other embodiments, the subject is a mammal.

As used herein, a “test subject,” typically, is any subject that is not in a training population used to construct a decision rule. A test subject can optionally be suspected of having an affective disorder or a likelihood of developing an affective disorder.

As used herein, a “training population” is a set of samples from a population of subjects used to construct a decision rule, using a data analysis algorithm, for evaluation of the biomarker profiles of subjects at risk of having an affective disorder. In a preferred embodiment, a training population includes samples from subjects that have an affective disorder and subjects that do not have an affective disorder.

As used herein, a “validation population” is a set of samples from a population of subjects used to determine the accuracy, or other performance metric, of a decision rule. In a preferred embodiment, a validation population includes samples from subjects that have an affective disorder and subjects that do not have an affective disorder. In a preferred embodiment, a validation population does not include subjects that are part of the training population used to train the decision rule for which an accuracy, or other performance metric, is sought.

As used herein, a “value set” is a combination of values, or ranges of values for features in a biomarker profile. The nature of this value set and the values therein is dependent upon the type of features present in the biomarker profile and the data analysis algorithm used to construct the decision rule that dictates the value set. To illustrate, reconsider exemplary biomarker profile 2:

### Exemplary Biomarker Profile 2

| | Feature |
|---|---|
| | Abundance in sample |
| Biomarker | in relative units |
| transcript of gene A | 300 |
| transcript of gene B | 400 |

In this example, the biomarker profile of each member of a training population is obtained. Each such biomarker profile includes a measured feature, here abundance, for the transcript of gene A, and a measured feature, here abundance, for the transcript of gene B. These feature values, here abundance values, are used by a data analysis algorithm to construct a decision rule. In this example, the data analysis algorithm is a decision tree, described below, and the final product of this data analysis algorithm, the decision rule, is a decision tree. The decision rule defines value sets. One such value set is predictive of an affective disorder. A subject whose biomarker feature values satisfy this value set has the affective disorder. An exemplary value set of this class is exemplary value set 1:

### Exemplary Value Set 1

| | Value set component |
|---|---|
| | (Abundance in sample |
| Biomarker | in relative units) |
| transcript of gene A | <400 |
| transcript of gene B | <600 |

Another such value set is predictive of an affective disorder free state. A subject whose biomarker feature values satisfy this value set is not diagnosed as having an affective disorder. An exemplary value set of this class is exemplary value set 2:

### Exemplary Value Set 2

| | Value set component |
|---|---|
| | (Abundance in sample |
| Biomarker | in relative units) |
| transcript of gene A | >400 |
| transcript of gene B | >600 |

In the case where the data analysis algorithm is a neural network analysis and the final product of this neural network analysis is an appropriately weighted neural network, one value set is those ranges of biomarker profile feature values that will cause the weighted neural network to indicate that a subject has an affective disorder. Another value set is those ranges of biomarker profile feature values that will cause the weighted neural network to indicate that a subject does not have an affective disorder.

As used herein, the term “probe spot” in the context of a microarray refers to a single stranded DNA molecule (e.g., a single stranded cDNA molecule or synthetic DNA oligomer), referred to herein as a “probe,” that is used to determine the abundance of a particular nucleic acid in a sample. For example, a probe spot can be used to determine the level of mRNA in a biological sample (e.g., a collection of cells) from a test subject. In a specific embodiment, a typical microarray comprises multiple probe spots that are placed onto a glass slide (or other substrate) in known locations on a grid. The nucleic acid for each probe spot is a single stranded contiguous portion of the sequence of a gene or gene of interest (e.g., a 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer or larger) and is a probe for the mRNA encoded by the particular gene or gene of interest. Each probe spot is characterized by a single nucleic acid sequence, and is hybridized under conditions that cause it to hybridize only to its complementary DNA strand or mRNA molecule. As such, there can be many probe spots on a substrate, and each can represent a unique gene or sequence of interest. In addition, two or more probe spots can represent the same gene sequence. In some embodiments, a labeled nucleic sample is hybridized to a probe spot, and the amount of labeled nucleic acid specifically hybridized to a probe spot can be quantified to determine the levels of that specific nucleic acid (e.g., mRNA transcript of a particular gene) in a particular biological sample. Probes, probe spots, and microarrays, generally, are described in Draghici, 2003, Data Analysis Tools for DNA Microarrays, Chapman & Hall/CRC, Chapter, 2.

### 5.2 Methods for Screening Subjects

The present invention allows for accurate, rapid prediction and/or diagnosis of affective disorders through detection of two or more features of a biomarker profile of a test individual suspected of having an affective disorder in a biological sample from the individual.

In specific embodiments of the invention, subjects suspected of having an affective disorder are screened using the methods of the present invention. In accordance with these embodiments, the methods of the present invention can be employed to screen, for example, subjects admitted to a psychiatric ward and/or those who have experienced some sort of psychological trauma.

In specific embodiments, a biological sample such as, for example, blood, is taken. In some embodiments, a biological sample is blood, a cerebrospinal fluid, a peritoneal fluid, an interstitial fluid, red blood cells, white blood cells or platelets. White blood cells (leukocytes) include, but are not limited to: neutrophils, basophils, eosinophils, lymphocytes, monocytes and macrophages. In some embodiments a biological sample is some component of whole blood. In one embodiment, present invention utilizes whole blood sampling with ready-to-use collection tubes containing an RNA stabilizer or preservative. This protocol is proven and ensures very little variability, provided the proper sample handling procedures are followed. The present invention provides reliable and robust transcriptional markers that can be used in high throughput analysis for large sample sets. This reliable method is shown to differentiate controls and patients. In some embodiments some portion of the mixture of proteins, nucleic acid, and/or other molecules (e.g., metabolites) within a cellular fraction or within a liquid (e.g., plasma or serum fraction) of the blood is resolved as a biomarker profile. This can be accomplished by measuring features of the biomarkers in the biomarker profile. In some embodiments, the biological sample is whole blood but the biomarker profile is resolved from biomarkers expressed or otherwise found in white blood cells that are isolated from the whole blood. In some embodiments, the biological sample is whole blood but the biomarker profile is resolved from biomarkers expressed or otherwise found in red blood cells that are isolated from the whole blood.

A biomarker profile can comprise at least two biomarkers, where the biomarkers can be in the same or different classes, such as, for example, a nucleic acid and a carbohydrate. In some embodiments, a biomarker profile comprises at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 96, 100, 105, 110, 115, 120, 125, 130, 135, 140, 145, 150, 155, 160, 165, 170, 175, 180, 185, 190, 195 or 200 or more biomarkers. In one embodiment, a biomarker profile comprises hundreds, or even thousands, of biomarkers. In some embodiments, a biomarker profile comprises at least 2, 3, 4, 5, 6, 7, 8, 9, 10, 11, 12, 13, 14, 15, 16, 17, 18, 19, 20, 25, 30, 35, 40, 45, 50, or more biomarkers. In one example, in some embodiments, a biomarker profile comprises at least 2, 3, 4, 5, 6, 7, 8, 9, 10, 11, 12, 13, 14, 15, 16, 17, 18, 19, 20, or more biomarkers selected from Table 1A.

In typical embodiments, each biomarker in the biomarker profile is represented by a feature. In other words, there is a correspondence between biomarkers and features. In some embodiments, the correspondence between biomarkers and features is 1:1, meaning that for each biomarker there is a feature. In some embodiments, there is more than one feature for each biomarker. In some embodiments the number of features corresponding to one biomarker in the biomarker profile is different than then number of features corresponding to another biomarker in the biomarker profile. As such, in some embodiments, a biomarker profile can include at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 96, 100, 105, 110, 115, 120, 125, 130, 135, 140, 145, 150, 155, 160, 165, 170, 175, 180, 185, 190, 195 or 200 or more features, provided that there are at least 2, 3, 4, 5, 6, or 7 or more biomarkers in the biomarker profile. In some embodiments, a biomarker profile can include at least 2, 3, 4, 5, 6, 7, 8, 9, 10, 11, 12, 13, 14, 15, 16, 17, 18, 19, 20, 25, 30, 35, 40, 45, 50, or more features. Regardless of embodiment, these features can be determined through the use of any reproducible measurement technique or combination of measurement techniques. Such techniques include those that are well known in the art including any technique described herein or, for example, any technique disclosed in Section 5.4, infra. Typically, such techniques are used to measure feature values using a biological sample taken from a subject at a single point in time or multiple samples taken at multiple points in time. In one embodiment, an exemplary technique to obtain a biomarker profile from a sample taken from a subject is a cDNA microarray (see, e.g., Section 5.4.1.2, infra). In another embodiment, an exemplary technique to obtain a biomarker profile from a sample taken from a subject is a protein-based assay or other form of protein-based technique such as described in the BD Cytometric Bead Array (CBA) Human Inflammation Kit Instruction Manual (BD Biosciences) or the bead assay described in U.S. Pat. No. 5,981,180, each of which is incorporated herein by reference in their entirety, and in particular for their teachings of various methods of assay protein concentrations in biological samples. In still another embodiment, the biomarker profile is mixed, meaning that it comprises some biomarkers that are nucleic acids, or indications thereof, and some biomarkers that are proteins, or indications thereof. In such embodiments, both protein based and nucleic acid based techniques are used to obtain a biomarker profile from one or more samples taken from a subject. In other words, the feature values for the features associated with the biomarkers in the biomarker profile that are nucleic acids are obtained by nucleic acid based measurement techniques (e.g., a nucleic acid microarray) and the feature values for the features associated with the biomarkers in the biomarker profile that are proteins are obtained by protein based measurement techniques. In some embodiments biomarker profiles can be obtained using a kit, such as a kit described in Section 5.3 below.

### 5.3 Kits

The invention also provides kits that are useful in diagnosing an affective disorder in a subject. In some embodiments, the kits of the present invention comprise at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 96, 100, 105, 110, 115, 120, 125, 130, 135, 140, 145, 150, 155, 160, 165, 170, 175, 180, 185, 190, 195 or 200 or more biomarkers and/or reagents to detect the presence or abundance of such biomarkers. In other embodiments, the kits of the present invention comprise at least 2, but as many as several hundred or more biomarkers. In some embodiments, the kits of the present invention comprise at least 2, 3, 4, 5, 6, 7, 8, 9, 10, 11, 12, 13, 14, 15, 16, 17, 18, 19, 20 or more biomarkers selected from Table 1A, or reagents to detect the presence or abundance of such biomarkers. In accordance with the definition of biomarkers given in Section 5.1, in some instances, a biomarker is in fact a discriminating molecule of, for example, a gene, mRNA, or protein rather than the gene, mRNA, or protein itself. Thus, a biomarker can be a molecule that indicates the presence or abundance of a particular gene, mRNA or protein, or fragment thereof, identified in Table 1A rather than the actual gene, mRNA or protein itself. In some embodiments, the kits of the present invention comprise at least 2, but as many as several hundred or more biomarkers. In some embodiments, at least twenty-five percent, at least thirty percent, at least thirty-five percent, at least forty percent, at least sixty percent, at least eighty percent of the biomarkers and/or reagents to detect the presence or abundance of the biomarkers are selected from the biomarkers from Table 1A and/or reagents to detect the presence or abundance of biomarkers selected from Table 1A.

The biomarkers of the kits of the present invention can be used to generate biomarker profiles according to the present invention. Examples of classes of compounds of the kit include, but are not limited to, proteins and fragments thereof, peptides, proteoglycans, glycoproteins, lipoproteins, carbohydrates, lipids, nucleic acids (e.g., DNA, such as cDNA or amplified DNA, or RNA, such as mRNA), organic or inorganic chemicals, natural or synthetic polymers, small molecules (e.g., metabolites), or discriminating molecules or discriminating fragments of any of the foregoing. In a specific embodiment, a biomarker is of a particular size, (e.g., at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 1000, 2000, 3000, 5000, 10 k, 20 k, 100 k Daltons or greater). The biomarker(s) may be part of an array, or the biomarker(s) may be packaged separately and/or individually. The kit may also comprise at least one internal standard to be used in generating the biomarker profiles of the present invention. Likewise, the internal standard or standards can be any of the classes of compounds described above.

In one embodiment, the invention provides kits comprising probes and/or primers that may or may not be immobilized at an addressable position on a substrate, such as found, for example, in a microarray. In a particular embodiment, the invention provides such a microarray.

In some embodiments of the invention, a kit may comprise a specific biomarker binding component, such as an aptamer. If the biomarkers comprise a nucleic acid, the kit may provide an oligonucleotide probe that is capable of forming a duplex with the biomarker or with a complementary strand of a biomarker. The oligonucleotide probe may be detectably labeled. In such embodiments, the probes are themselves biomarkers that fall within the scope of the present invention.

The kits of the present invention may also include additional compositions, such as buffers, that can be used in constructing the biomarker profile. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

Some kits of the present invention comprise a microarray. In one embodiment this microarray comprises a plurality of probe spots, wherein at least twenty percent of the probe spots in the plurality of probe spots correspond to biomarkers in Table 1A. In some embodiments, at least twenty-five percent, at least thirty percent, at least thirty-five percent, at least forty percent, at least sixty percent, or at least eighty percent of the probe spots in the plurality of probe spots correspond to biomarkers in Table 1A, and/or reagents to detect the presence on abundance of biomarkers in Table 1A. Such probe spots are biomarkers within the scope of the present invention. In some embodiments, the microarray consists of between about two and about one hundred probe spots on a substrate. In some embodiments, the microarray consists of between about two and about one hundred probe spots on a substrate. As used in this context, the term “about” means within five percent of the stated value, within ten percent of the stated value, or within twenty-five percent of the stated value. In some embodiments, such microarrays contain one or more probe spots for inter-microarray calibration or for calibration with other microarrays such as reference microarrays using techniques that are known to those of skill in the art. In some embodiments such microarrays are nucleic acid microarrays. In some embodiments, such microarrays are protein microarrays.

Some kits of the present invention are implemented as a computer program product that comprises a computer program mechanism embedded in a computer-readable storage medium. Further, any of the methods of the present invention can be implemented in one or more computers or other forms of apparatus. Examples of apparatus include but are not limited to, a computer, and a spectroscopic measuring device (e.g., a microarray reader or microarray scanner). Further still, any of the methods of the present invention can be implemented in one or more computer program products. Some embodiments of the present invention provide a computer program product that encodes any or all of the methods disclosed herein. Such methods can be stored on a CD-ROM, DVD, magnetic disk storage product, or any other tangible computer-readable data or tangible program storage product. Such methods can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices. Such methods encoded in the computer program product can also be distributed electronically, via the Internet or otherwise.

Some kits of the present invention provide a computer program product that contains one or more programs that individually or collectively carry out any of the methods of the present invention. These program modules can be stored on a CD-ROM, DVD, magnetic disk storage product, or any other tangible computer-readable data or program storage product. The program modules can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices. The software modules in the computer program product can also be distributed electronically, via the Internet or otherwise.

Some kits of the present invention comprise a computer having one or more processing units and a memory coupled to the one or more processing units. The memory stores instructions for evaluating whether a plurality of features in a biomarker profile of a test subject at risk for having an affective disorder satisfies a value set. In some embodiments, satisfying the value set diagnoses the subject as having an affective disorder. In some embodiments, satisfying the value set diagnoses the subject as not having an affective disorder. In one embodiment, the plurality of features corresponds to biomarkers listed in Table 1A.

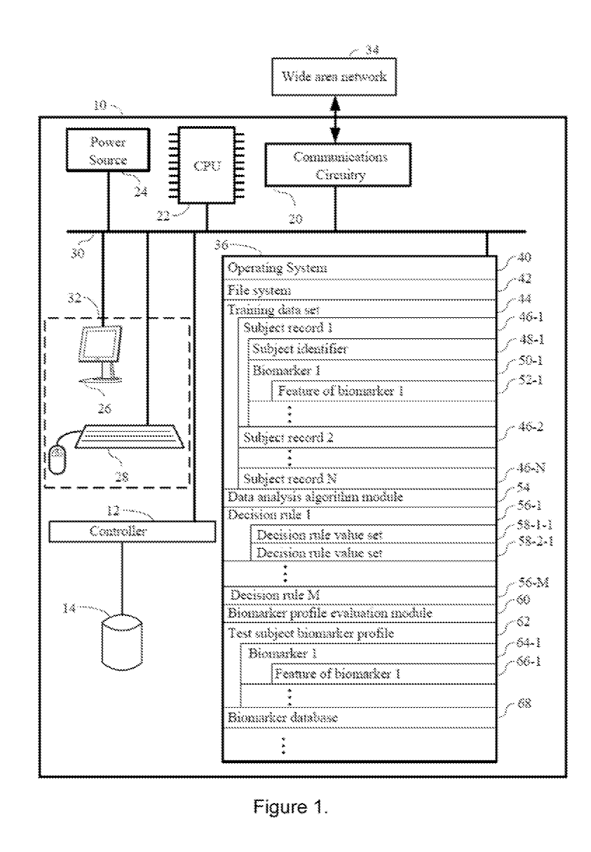

FIG. 1 details an exemplary system that supports the functionality described above. The system is preferably a computer system 10 having:
a central processing unit 22;
a main non-volatile storage unit 14, for example, a hard disk drive, for storing software and data, the storage unit 14 controlled by storage controller 12;
a system memory 36, preferably high speed random-access memory (RAM), for storing system control programs, data, and application programs, comprising programs and data loaded from non-volatile storage unit 14; system memory 36 may also include read-only memory (ROM);
a user interface 32, comprising one or more input devices (e.g., keyboard 28) and a display 26 or other output device;
a network interface card 20 for connecting to any wired or wireless communication network 34 (e.g., a wide area network such as the Internet);
an internal bus 30 for interconnecting the aforementioned elements of the system; and
a power source 24 to power the aforementioned elements.

Operation of computer 10 is controlled primarily by operating system 40, which is executed by central processing unit 22. Operating system 40 can be stored in system memory 36. In addition to operating system 40, in a typical implementation, system memory 36 includes:
file system 42 for controlling access to the various files and data structures used by the present invention;
a training data set 44 for use in construction one or more decision rules in accordance with the present invention;
a data analysis algorithm module 54 for processing training data and constructing decision rules;
one or more decision rules 56;
a biomarker profile evaluation module 60 for determining whether a plurality of features in a biomarker profile of a test subject satisfies a first value set or a second value set;
a test subject biomarker profile 62 comprising biomarkers 64 and, for each such biomarkers, features 66; and
a database 68 of select biomarkers of the present invention (e.g., Table 1A) and/or one or features for each of these select biomarkers.

Training data set 46 comprises data for a plurality of subjects 46. For each subject 46 there is a subject identifier 48 and a plurality of biomarkers 50. For each biomarker 50, there is at least one feature 52. Although not shown in FIG. 1, for each feature 52, there is a feature value. For each decision rule 56 constructed using data analysis algorithms, there is at least one decision rule value set 58.

As illustrated in FIG. 1, computer 10 comprises software program modules and data structures. The data structures stored in computer 10 include training data set 44, decision rules 56, test subject biomarker profile 62, and biomarker database 68. Each of these data structures can comprise any form of data storage system including, but not limited to, a flat ASCII or binary file, an Excel spreadsheet, a relational database (SQL), or an on-line analytical processing (OLAP) database (MDX and/or variants thereof). In some specific embodiments, such data structures are each in the form of one or more databases that include hierarchical structure (e.g., a star schema). In some embodiments, such data structures are each in the form of databases that do not have explicit hierarchy (e.g., dimension tables that are not hierarchically arranged).

In some embodiments, each of the data structures stored or accessible to system 10 are single data structures. In other embodiments, such data structures in fact comprise a plurality of data structures (e.g., databases, files, archives) that may or may not all be hosted by the same computer 10. For example, in some embodiments, training data set 44 comprises a plurality of Excel spreadsheets that are stored either on computer 10 and/or on computers that are addressable by computer 10 across wide area network 34. In another example, training data set 44 comprises a database that is either stored on computer 10 or is distributed across one or more computers that are addressable by computer 10 across wide area network 34.

It will be appreciated that many of the modules and data structures illustrated in FIG. 1 can be located on one or more remote computers. For example, some embodiments of the present application are web service-type implementations. In such embodiments, biomarker profile evaluation module 60 and/or other modules can reside on a client computer that is in communication with computer 10 via network 34. In some embodiments, for example, biomarker profile evaluation module 60 can be an interactive web page.

In some embodiments, training data set 44, decision rules 56, and/or biomarker database 68 illustrated in FIG. 1 are on a single computer (computer 10) and in other embodiments one or more of such data structures and modules are hosted by one or more remote computers (not shown). Any arrangement of the data structures and software modules illustrated in FIG. 1 on one or more computers is within the scope of the present invention so long as these data structures and software modules are addressable with respect to each other across network 34 or by other electronic means. Thus, the present invention fully encompasses a broad array of computer systems.

Still another embodiment of the present invention provides a graphical user interface for determining whether a subject has an affective disorder. The graphical user interface comprises a display field for a displaying a result encoded in a digital signal embodied on a carrier wave received from a remote computer. The plurality of features are measurable aspects of a plurality of biomarkers. The plurality of biomarkers comprise at least two biomarkers listed in Table 1A. The result has a first value when a plurality of features in a biomarker profile of a test subject satisfies a first value set. The result has a second value when a plurality of features in a biomarker profile of a test subject satisfies a second value set.

### 5.4 Generation of Biomarker Profiles

According to one embodiment, the methods of the present invention comprise generating a biomarker profile from a biological sample taken from a subject. The biological sample may be, for example, a peripheral tissue, whole blood, a cerebrospinal fluid, a peritoneal fluid, an interstitial fluid, red blood cells, white blood cells or platelets.

### 5.4.1 Methods of Detecting Nucleic Acid Biomarkers

In specific embodiments of the invention, biomarkers in a biomarker profile are nucleic acids. Such biomarkers and corresponding features of the biomarker profile may be generated, for example, by detecting the expression product (e.g., a polynucleotide or polypeptide) of one or more genes described herein (e.g., a gene listed in Table 1A). In a specific embodiment, the biomarkers and corresponding features in a biomarker profile are obtained by detecting and/or analyzing one or more nucleic acids expressed from a gene disclosed herein (e.g., a gene listed in Table 1A) using any method well known to those skilled in the art including, but by no means limited to, hybridization, microarray analysis, RT-PCR, nuclease protection assays and Northern blot analysis.

In certain embodiments, nucleic acids detected and/or analyzed by the methods and compositions of the invention include RNA molecules such as, for example, expressed RNA molecules which include messenger RNA (mRNA) molecules, mRNA spliced variants as well as regulatory RNA, cRNA molecules (e.g., RNA molecules prepared from cDNA molecules that are transcribed in vitro) and discriminating fragments thereof. Nucleic acids detected and/or analyzed by the methods and compositions of the present invention can also include, for example, DNA molecules such as genomic DNA molecules, cDNA molecules, and discriminating fragments thereof (e.g., oligonucleotides, ESTs, STSs, etc.).

The nucleic acid molecules detected and/or analyzed by the methods and compositions of the invention may be naturally occurring nucleic acid molecules such as genomic or extragenomic DNA molecules isolated from a sample, or RNA molecules, such as mRNA molecules, present in, isolated from or derived from a biological sample. The sample of nucleic acids detected and/or analyzed by the methods and compositions of the invention comprise, e.g., molecules of DNA, RNA, or copolymers of DNA and RNA. Generally, these nucleic acids correspond to particular genes or alleles of genes, or to particular gene transcripts (e.g., to particular mRNA sequences expressed in specific cell types or to particular cDNA sequences derived from such mRNA sequences). The nucleic acids detected and/or analyzed by the methods and compositions of the invention may correspond to different exons of the same gene, e.g., so that different splice variants of that gene may be detected and/or analyzed.

In specific embodiments, the nucleic acids are prepared in vitro from nucleic acids present in, or isolated or partially isolated from biological a sample. For example, in one embodiment, RNA is extracted from a sample (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., 2001, Molecular Cloning: A Laboratory Manual. 3^{rd} ed. Cold Spring Harbor Laboratory Press (Cold Spring Harbor, N.Y.).

### 5.4.1.1 Nucleic Acid Arrays

In certain embodiments of the invention, nucleic acid arrays are employed to generate features of biomarkers in a biomarker profile by detecting the expression of any one or more of the genes described herein (e.g., a gene listed in Table 1A). In one embodiment of the invention, a microarray, such as a cDNA microarray, is used to determine feature values of biomarkers in a biomarker profile. The diagnostic use of cDNA arrays is well known in the art. (See, e.g., Zou et. al., 2002, Oncogene 21:4855-4862; as well as Draghici, 2003, Data Analysis Tools for DNA Microarrays, Chapman & Hall/CRC). Exemplary methods for cDNA microarray analysis are described below.

In certain embodiments, the feature values for biomarkers in a biomarker profile are obtained by hybridizing to the array detectably labeled nucleic acids representing or corresponding to the nucleic acid sequences in mRNA transcripts present in a biological sample (e.g., fluorescently labeled cDNA synthesized from the sample) to a microarray comprising one or more probe spots.

Nucleic acid arrays, for example, microarrays, can be made in a number of ways, of which several are described herein below. Preferably, the arrays are reproducible, allowing multiple copies of a given array to be produced and results from said microarrays compared with each other. Preferably, the arrays are made from materials that are stable under binding (e.g., nucleic acid hybridization) conditions. Those skilled in the art will know of suitable supports, substrates or carriers for hybridizing test probes to probe spots on an array, or will be able to ascertain the same by use of routine experimentation.

Arrays, for example, microarrays, used can include one or more test probes. In some embodiments each such test probe comprises a nucleic acid sequence that is complementary to a subsequence of RNA or DNA to be detected. Each probe typically has a different nucleic acid sequence, and the position of each probe on the solid surface of the array is usually known or can be determined. Arrays useful in accordance with the invention can include, for example, oligonucleotide microarrays, cDNA based arrays, SNP arrays, spliced variant arrays and any other array able to provide a qualitative, quantitative or semi-quantitative measurement of expression of a gene described herein (e.g., a gene listed in Table 1A). Some types of microarrays are addressable arrays. More specifically, some microarrays are positionally addressable arrays. In some embodiments, each probe of the array is located at a known, predetermined position on the solid support so that the identity (e.g., the sequence) of each probe can be determined from its position on the array (e.g., on the support or surface). In some embodiments, the arrays are ordered arrays. Microarrays are generally described in Draghici, 2003, Data Analysis Tools for DNA Microarrays, Chapman & Hall/CRC.

In some embodiments of the present invention, an expressed transcript (e.g., a transcript of a gene described herein) is represented in the nucleic acid arrays. In such embodiments, a set of binding sites can include probes with different nucleic acids that are complementary to different sequence segments of the expressed transcript. Exemplary nucleic acids that fall within this class can be of length of 15 to 200 bases, 20 to 100 bases, 25 to 50 bases, 40 to 60 bases or some other range of bases. Each probe sequence can also comprise one or more linker sequences in addition to the sequence that is complementary to its target sequence. As used herein, a linker sequence is a sequence between the sequence that is complementary to its target sequence and the surface of support. For example, the nucleic acid arrays of the invention can comprise one probe specific to each target gene or exon. However, if desired, the nucleic acid arrays can contain at least 2, 5, 10, 100, or 1000 or more probes specific to some expressed transcript (e.g., a transcript of a gene described herein, e.g., in Table 1A). For example, the array may contain probes tiled across the sequence of the longest mRNA isoform of a gene.

It will be appreciated that when cDNA complementary to the RNA of a cell, for example, a cell in a biological sample, is made and hybridized to a microarray under suitable hybridization conditions, the level of hybridization to the site in the array corresponding to a gene described herein (e.g., a gene listed in Table 1A) will reflect the prevalence in the cell of mRNA or mRNAs transcribed from that gene. Alternatively, in instances where multiple isoforms or alternate splice variants produced by particular genes are to be distinguished, detectably labeled (e.g., with a fluorophore) cDNA complementary to the total cellular mRNA can be hybridized to a microarray, and the site on the array corresponding to an exon of the gene that is not transcribed or is removed during RNA splicing in the cell will have little or no signal (e.g., fluorescent signal), and a site corresponding to an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In one embodiment, hybridization levels at different hybridization times are measured separately on different, identical microarrays. For each such measurement, at hybridization time when hybridization level is measured, the microarray is washed briefly, preferably in room temperature in an aqueous solution of high to moderate salt concentration (e.g., 0.5 to 3 M salt concentration) under conditions which retain all bound or hybridized nucleic acids while removing all unbound nucleic acids. The detectable label on the remaining, hybridized nucleic acid molecules on each probe is then measured by a method which is appropriate to the particular labeling method used. The resulting hybridization levels are then combined to form a hybridization curve. In another embodiment, hybridization levels are measured in real time using a single microarray. In this embodiment, the microarray is allowed to hybridize to the sample without interruption and the microarray is interrogated at each hybridization time in a non-invasive manner. In still another embodiment, one can use one array, hybridize for a short time, wash and measure the hybridization level, put back to the same sample, hybridize for another period of time, wash and measure again to get the hybridization time curve.

In some embodiments, nucleic acid hybridization and wash conditions are chosen so that the nucleic acid biomarkers to be analyzed specifically bind or specifically hybridize to the complementary nucleic acid sequences of the array, typically to a specific array site, where its complementary DNA is located.

Arrays containing double-stranded probe DNA situated thereon can be subjected to denaturing conditions to render the DNA single-stranded prior to contacting with the target nucleic acid molecules. Arrays containing single-stranded probe DNA (e.g., synthetic oligodeoxyribonucleic acids) may need to be denatured prior to contacting with the target nucleic acid molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., latest edition, Current Protocols in Molecular Biology, Greene Publishing and Wiley-Interscience, New York. When the cDNA microarrays of Shena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization With Nucleic Acid Probes, Elsevier Science Publishers B.V.; Kricka, 1992, Nonisotopic DNA Probe Techniques, Academic Press, San Diego, Calif.; and Zou et. al., 2002, Oncogene 21:4855-4862; and Draghici, Data Analysis Tools for DNA Microanalysis, 2003, CRC Press LLC, Boca Raton, Fla., pp. 342-343.

In a specific embodiment, a microarray can be used to sort out RT-PCR products that have been generated by the methods described, for example, below in Section 5.4.1.2.

### 5.4.1.2 RT-PCR

In certain embodiments, to determine the feature values of biomarkers in a biomarker profile of the invention, the level of expression of one or more of the genes described herein (e.g., a gene listed in Table 1A) is measured by amplifying RNA from a sample using reverse transcription (RT) in combination with the polymerase chain reaction (PCR). In accordance with this embodiment, the reverse transcription may be quantitative or semi-quantitative. The RT-PCR methods taught herein may be used in conjunction with the microarray methods described above, for example, in Section 5.4.1.1. For example, a bulk PCR reaction may be performed, the PCR products may be resolved and used as probe spots on a microarray.

Total RNA, or mRNA from a sample is used as a template and a primer specific to the transcribed portion of the gene(s) is used to initiate reverse transcription. Methods of reverse transcribing RNA into cDNA are well known and described in Sambrook et al., 2001, supra. Primer design can be accomplished based on known nucleotide sequences that have been published or available from any publicly available sequence database such as GenBank. For example, primers may be designed for any of the genes described herein (see, e.g., in Table 1A). Further, primer design may be accomplished by utilizing commercially available software (e.g., Primer Designer 1.0, Scientific Software etc.). The product of the reverse transcription is subsequently used as a template for PCR.

PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts. The method of PCR is well known in the art. PCR, is performed, for example, as described in Mullis and Faloona, 1987, Methods Enzymol. 155:335.

PCR can be performed using template DNA or cDNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10 M PCR buffer 1 (Perkin-Elmer, Foster City, Calif.), 0.4 μl of 1.25 M dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

Quantitative RT-PCR (“QRT-PCR”), which is quantitative in nature, can also be performed to provide a quantitative measure of gene expression levels. In QRT-PCR reverse transcription and PCR can be performed in two steps, or reverse transcription combined with PCR can be performed concurrently. One of these techniques, for which there are commercially available kits such as Taqman (Perkin Elmer, Foster City, Calif.) or as provided by Applied Biosystems (Foster City, Calif.) is performed with a transcript-specific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96-well plates so that samples derived from many individuals are processed and measured simultaneously. The Taqman system has the additional advantage of not requiring gel electrophoresis and allows for quantification when used with a standard curve.

A second technique useful for detecting PCR products quantitatively is to use an intercolating dye such as the commercially available QuantiTect SYBR Green PCR (Qiagen, Valencia Calif.). RT-PCR is performed using SYBR green as a fluorescent label which is incorporated into the PCR product during the PCR stage and produces a flourescense proportional to the amount of PCR product.

Both Taqman and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other systems to quantitatively measure mRNA expression products are known including MOLECULAR BEACONS® which uses a probe having a fluorescent molecule and a quencher molecule, the probe capable of forming a hairpin structure such that when in the hairpin form, the fluorescence molecule is quenched, and when hybridized the fluorescence increases giving a quantitative measurement of gene expression.

Additional techniques to quantitatively measure RNA expression include, but are not limited to, polymerase chain reaction, ligase chain reaction, Qbeta replicase (see, e.g., International Application No. PCT/US87/00880), isothermal amplification method (see, e.g., Walker et al., 1992, PNAS 89:382-396), strand displacement amplification (SDA), repair chain reaction, Asymmetric Quantitative PCR (see, e.g., U.S. Publication No. US 2003/30134307A1) and the multiplex microsphere bead assay described in Fuja et al., 2004, Journal of Biotechnology 108:193-205.

### 5.4.2 Methods of Detecting Proteins

In specific embodiments of the invention, feature values of biomarkers in a biomarker profile can be obtained by detecting proteins, for example, by detecting the expression product (e.g., a nucleic acid or protein) of one or more genes described herein (e.g., a gene listed in Table 1A), or post-translationally modified, or otherwise modified, or processed forms of such proteins. In a specific embodiment, a biomarker profile is generated by detecting and/or analyzing one or more proteins and/or discriminating fragments thereof expressed from a gene disclosed herein (e.g., a gene listed in Table 1A) using any method known to those skilled in the art for detecting proteins including, but not limited to protein microarray analysis, immunohistochemistry and mass spectrometry.

Standard techniques may be utilized for determining the amount of the protein or proteins of interest (e.g., proteins expressed from genes listed in Table 1A) present in a sample. For example, standard techniques can be employed using, e.g., immunoassays such as, for example Western blot, immunoprecipitation followed by sodium dodecyl sulfate polyacrylamide gel electrophoresis, (SDS-PAGE), immunocytochemistry, and the like to determine the amount of protein or proteins of interest present in a sample. One exemplary agent for detecting a protein of interest is an antibody capable of specifically binding to a protein of interest, preferably an antibody detectably labeled, either directly or indirectly.

For such detection methods, if desired a protein from the sample to be analyzed can easily be isolated using techniques which are well known to those of skill in the art. Protein isolation methods can, for example, be such as those described in Harlow and Lane, 1988, Antibodies: A Laboratory Manual, Cold Spring Harbor Laboratory Press (Cold Spring Harbor, N.Y.).

### 5.5 Data Analysis Algorithms

Biomarkers whose corresponding feature values are capable of diagnosing an affective disorder are identified in the present invention. The identity of these biomarkers and their corresponding features (e.g., expression levels) can be used to develop a decision rule, or plurality of decision rules, that discriminate between subjects that have an affective disorder and subjects that do not. Once a decision rule has been built using these exemplary data analysis algorithms or other techniques known in the art, the decision rule can be used to classify a test subject into one of the two or more phenotypic classes (e.g., has an affective disorder, does not have an affective disorder). This is accomplished by applying the decision rule to a biomarker profile obtained from the test subject. Such decision rules, therefore, have enormous value as diagnostic indicators.

The present invention provides, in one aspect, for the evaluation of a biomarker profile from a test subject to biomarker profiles obtained from a training population. In some embodiments, each biomarker profile obtained from subjects in the training population, as well as the test subject, comprises a feature for each of a plurality of different biomarkers. In some embodiments, this comparison is accomplished by (i) developing a decision rule using the biomarker profiles from the training population and (ii) applying the decision rule to the biomarker profile from the test subject. As such, the decision rules applied in some embodiments of the present invention are used to determine whether a test subject has an affective disorder.

In some embodiments of the present invention, when the results of the application of a decision rule indicate that the subject has an affective disorder, the subject is diagnosed as a “affective disorder” subject. If the results of an application of a decision rule indicate that the subject does not have the disorder, the subject is diagnosed as a “not affective disorder” subject. Thus, in some embodiments, the result in the above-described binary decision situation has four possible outcomes:
(i) truly has affective disorder, where the decision rule indicates that the subject has an affective disorder and the subject does in fact have the affective disorder (true positive, TP);
(ii) falsely has affective disorder, where the decision rule indicates that the subject has an affective disorder, but in fact, the subject does not have the affective disorder (false positive, FP);
(iii) truly does not have affective disorder, where the decision rule indicates that the subject does not have the an affective disorder and the subject, in fact, does not have the affective disorder (true negative, TN); or
(iv) falsely does not have the affective disorder, where the decision rule indicates that the subject does not have the affective disorder and the subject, in fact, does have the affective disorder (false negative, FN).

It will be appreciated that other definitions for TP, FP, TN, FN can be made. While all such alternative definitions are within the scope of the present invention, for ease of understanding the present invention, the definitions for TP, FP, TN, and FN given by definitions (i) through (iv) above will be used herein, unless otherwise stated.

As will be appreciated by those of skill in the art, a number of quantitative criteria can be used to communicate the performance of the comparisons made between a test biomarker profile and reference biomarker profiles (e.g., the application of a decision rule to the biomarker profile from a test subject). These include positive predicted value (PPV), negative predicted value (NPV), specificity, sensitivity, accuracy, and certainty. In addition, other constructs such a receiver operator curves (ROC) can be used to evaluate decision rule performance. As used herein:

$$PPV = \frac{TP}{TP + FP}$$

$$NPV = \frac{TN}{TN + FN}$$

$$specificity = \frac{TN}{TN + FP}$$

$$sensitivity = \frac{TP}{TP + FN}$$

$$accuracy = certainty = \frac{TP + TN}{N}$$

Here, N is the number of samples compared (e.g., the number of test samples). For example, consider the case in which there are ten subjects for which the affective disorder classification is sought. Biomarker profiles are constructed for each of the ten test subjects. Then, each of the biomarker profiles is evaluated by applying a decision rule, where the decision rule was developed based upon biomarker profiles obtained from a training population. In this example, N, from the above equations, is equal to 10. Typically, N is a number of samples, where each sample was collected from a different member of a population. This population can, in fact, be of two different types. In one type, the population comprises subjects whose samples and phenotypic data (e.g., feature values of biomarkers and an indication of whether or not the subject has the affective disorder) was used to construct or refine a decision rule. Such a population is referred to herein as a training population. In the other type, the population comprises subjects that were not used to construct the decision rule. Such a population is referred to herein as a validation population. Unless otherwise stated, the population represented by N is either exclusively a training population or exclusively a validation population, as opposed to a mixture of the two population types. It will be appreciated that scores such as accuracy will be higher (closer to unity) when they are based on a training population as opposed to a validation population. Nevertheless, unless otherwise explicitly stated herein, all criteria used to assess the performance of a decision rule (or other forms of evaluation of a biomarker profile from a test subject) including certainty (accuracy) refer to criteria that were measured by applying the decision rule corresponding to the criteria to either a training population or a validation population. Furthermore, the definitions for PPV, NPV, specificity, sensitivity, and accuracy defined above can also be found in Draghici, Data Analysis Tools for DNA Microanalysis, 2003, CRC Press LLC, Boca Raton, Fla., pp. 342-343.

In some embodiments, N is more than one, more than five, more than ten, more than twenty, between ten and 100, more than 100, or less than 1000 subjects. A decision rule (or other forms of comparison) can have at least about 99% certainty, or even more, in some embodiments, against a training population or a validation population. In other embodiments, the certainty is at least about 97%, at least about 95%, at least about 90%, at least about 85%, at least about 80%, at least about 75%, at least about 70%, at least about 65%, or at least about 60% against a training population or a validation population (and therefore against a single subject that is not part of a training population such as a clinical patient). The useful degree of certainty may vary, depending on the particular method of the present invention. As used herein, “certainty” means “accuracy.” In one embodiment, the sensitivity and/or specificity is at is at least about 97%, at least about 95%, at least about 90%, at least about 85%, at least about 80%, at least about 75%, or at least about 70% against a training population or a validation population. In some embodiments, such decision rules are used to predict whether a subject has an affective disorder with the stated accuracy. In some embodiments, such decision rules are used to diagnoses an affective disorder with the stated accuracy. In some embodiments, such decision rules are used to determine a likelihood that a subject has a symptom of an affective disorder with the stated accuracy.

The number of features that may be used by a decision rule to classify a test subject with adequate certainty is two or more. In some embodiments, it is three or more, four or more, ten or more, or between 10 and 200. Depending on the degree of certainty sought, however, the number of features used in a decision rule can be more or less, but in all cases is at least two. In one embodiment, the number of features that may be used by a decision rule to classify a test subject is optimized to allow a classification of a test subject with high certainty.

Relevant data analysis algorithms for developing a decision rule include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977); tree-based algorithms such as classification and regression trees (CART) and variants (see, e.g., Breiman, 1984, Classification and Regression Trees, Belmont, Calif.: Wadsworth International Group); generalized additive models (see, e.g., Tibshirani, 1990, Generalized Additive Models, London: Chapman and Hall); and neural networks (see, e.g., Neal, 1996, Bayesian Learning for Neural Networks, New York: Springer-Verlag; and Insua, 1998, Feedforward neural networks for nonparametric regression In: Practical Nonparametric and Semiparametric Bayesian Statistics, pp. 181-194, New York: Springer, as well as Section 5.5.2, below).

In one embodiment, comparison of a test subject's biomarker profile to a biomarker profiles obtained from a training population is performed, and comprises applying a decision rule. The decision rule is constructed using a data analysis algorithm, such as a computer pattern recognition algorithm. Other suitable data analysis algorithms for constructing decision rules include, but are not limited to, logistic regression or a nonparametric algorithm that detects differences in the distribution of feature values (e.g., a Wilcoxon Signed Rank Test (unadjusted and adjusted)). The decision rule can be based upon two, three, four, five, 10, 20 or more features, corresponding to measured observables from one, two, three, four, five, 10, 20 or more biomarkers. In one embodiment, the decision rule is based on hundreds of features or more. Decision rules may also be built using a classification tree algorithm. For example, each biomarker profile from a training population can comprise at least three features, where the features are predictors in a classification tree algorithm (see Section 5.5.1, below). The decision rule predicts membership within a population (or class) with an accuracy of at least about at least about 70%, of at least about 75%, of at least about 80%, of at least about 85%, of at least about 90%, of at least about 95%, of at least about 97%, of at least about 98%, of at least about 99%, or about 100%.

Suitable data analysis algorithms are known in the art, some of which are reviewed in Hastie et al., supra. In a specific embodiment, a data analysis algorithm of the invention comprises Classification and Regression Tree (CART; Section 5.5.1, below), Multiple Additive Regression Tree (MART), Prediction Analysis for Microarrays (PAM) or Random Forest analysis (Section 5.5.1, below). Such algorithms classify complex spectra from biological materials, such as a blood sample, to distinguish subjects as normal or as possessing biomarker expression levels characteristic of a particular disease state. In other embodiments, a data analysis algorithm of the invention comprises ANOVA and nonparametric equivalents, linear discriminant analysis, logistic regression analysis, nearest neighbor classifier analysis, neural networks (Section 5.5.2, below), principal component analysis, quadratic discriminant analysis, regression classifiers and support vector machines (Section 5.5.4, below), relevance vector machines and genetic algorithms (Section 5.5.5, below). While such algorithms may be used to construct a decision rule and/or increase the speed and efficiency of the application of the decision rule and to avoid investigator bias, one of ordinary skill in the art will realize that computer-based algorithms are not required to carry out the methods of the present invention.

Decision rules can be used to evaluate biomarker profiles, regardless of the method that was used to generate the biomarker profile. For example, suitable decision rules that can be used to evaluate biomarker profiles generated using gas chromatography, as discussed in Harper, “Pyrolysis and GC in Polymer Analysis,” Dekker, New York (1985). Further, Wagner et al., 2002, Anal. Chem. 74:1824-1835 disclose a decision rule that improves the ability to classify subjects based on spectra obtained by static time-of-flight secondary ion mass spectrometry (TOF-SIMS). Additionally, Bright et al., 2002, J. Microbiol. Methods 48:127-38, disclose a method of distinguishing between bacterial strains with high certainty (79-89% correct classification rates) by analysis of MALDI-TOF-MS spectra. Dalluge, 2000, Fresenius J. Anal. Chem. 366:701-711, discusses the use of MALDI-TOF-MS and liquid chromatography-electrospray ionization mass spectrometry (LC/ESI-MS) to classify profiles of biomarkers in complex biological samples.

### 5.5.1 Decision Trees

One type of decision rule that can be constructed using the feature values of the biomarkers identified in the present invention is a decision tree. Here, the “data analysis algorithm” is any technique that can build the decision tree, whereas the final “decision tree” is the decision rule. A decision tree is constructed using a training population and specific data analysis algorithms. Decision trees are described generally by Duda, 2001, Pattern Classification, John Wiley & Sons, Inc., New York. pp. 395-396. Tree-based methods partition the feature space into a set of rectangles, and then fit a model (like a constant) in each one.

The training population data includes the features (e.g., expression values, or some other observable) for the biomarkers of the present invention across a training set population. One specific algorithm that can be used to construct a decision tree is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412. CART, MART, and C4.5 are described in Hastie et al., 2001, The Elements of Statistical Learning, Springer-Verlag, New York, Chapter 9. Random Forests are described in Breiman, 1999, “Random Forests—Random Features,” Technical Report 567, Statistics Department, U.C. Berkeley, September 1999.

In some embodiments of the present invention, decision trees are used to classify subjects using features for combinations of biomarkers of the present invention. Decision tree algorithms belong to the class of supervised learning algorithms. The aim of a decision tree is to induce a classifier (a tree) from real-world example data. This tree can be used to classify unseen examples that have not been used to derive the decision tree. As such, a decision tree is derived from training data. Exemplary training data contains data for a plurality of subjects (the training population). For each respective subject there is a plurality of features the class of the respective subject (e.g., has affective disorder/does not have affective disorder). In one embodiment of the present invention, the training data is expression data for a combination of biomarkers across the training population.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, but are not limited to classification and regression trees (CART), multivariate decision trees, ID3, and C4.5.

In one approach, when a decision tree is used, the gene expression data for a select combination of genes described in the present invention across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of biomarkers described in the present invention is used to construct the decision tree. Then, the ability for the decision tree to correctly classify members in the test set is determined. In some embodiments, this computation is performed several times for a given combination of biomarkers. In each computational iteration, the members of the training population are randomly assigned to the training set and the test set. Then, the quality of the combination of biomarkers is taken as the average of each such iteration of the decision tree computation.

In addition to univariate decision trees in which each split is based on a feature value for a corresponding biomarker, among the set of biomarkers of the present invention, or the relative feature values of two such biomarkers, multivariate decision trees can be implemented as a decision rule. In such multivariate decision trees, some or all of the decisions actually comprise a linear combination of feature values for a plurality of biomarkers of the present invention. Such a linear combination can be trained using known techniques such as gradient descent on a classification or by the use of a sum-squared-error criterion. To illustrate such a decision tree, consider the expression:

0.04x₁+0.16x₂<500

Here, X₁ and X₂ refer to two different features for two different biomarkers from among the biomarkers of the present invention. To poll the decision rule, the values of features X₁ and X₂ are obtained from the measurements obtained from the unclassified subject. These values are then inserted into the equation. If a value of less than 500 is computed, then a first branch in the decision tree is taken. Otherwise, a second branch in the decision tree is taken. Multivariate decision trees are described in Duda, 2001, Pattern Classification, John Wiley & Sons, Inc., New York, pp. 408-409.

Another approach that can be used in the present invention is multivariate adaptive regression splines (MARS). MARS is an adaptive procedure for regression, and is well suited for the high-dimensional problems addressed by the present invention. MARS can be viewed as a generalization of stepwise linear regression or a modification of the CART method to improve the performance of CART in the regression setting. MARS is described in Hastie et al., 2001, The Elements of Statistical Learning, Springer-Verlag, New York, pp. 283-295.

### 5.5.2 Neural Networks

In some embodiments, the feature data measured for select biomarkers of the present invention (e.g., RT-PCR data, mass spectrometry data, microarray data) can be used to train a neural network. A neural network is a two-stage regression or classification decision rule. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, New York. Neural networks are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, N.Y. What is disclosed below is some exemplary forms of neural networks.

The basic approach to the use of neural networks is to start with an untrained network, present a training pattern to the input layer, and to pass signals through the net and determine the output at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the classifier defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear classifier. In some embodiments, starting values for weights are chosen to be random values near zero. Hence the classifier starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range [−0.7, +0.7].

A recurrent problem in the use of three-layer networks is the optimal number of hidden units to use in the network. The number of inputs and outputs of a three-layer network are determined by the problem to be solved. In the present invention, the number of inputs for a given neural network will equal the number of biomarkers selected from the training population. The number of output for the neural network will typically be just one. However, in some embodiments more than one output is used so that more than just two states can be defined by the network. For example, a multi-output neural network can be used to discriminate between, healthy phenotypes, various stages of an affective disorder. If too many hidden units are used in a neural network, the network will have too many degrees of freedom and is trained too long, there is a danger that the network will overfit the data. If there are too few hidden units, the training set cannot be learned. Generally speaking, however, it is better to have too many hidden units than too few. With too few hidden units, the classifier might not have enough flexibility to capture the nonlinearities in the date; with too many hidden units, the extra weight can be shrunk towards zero if appropriate regularization or pruning, as described below, is used. In typical embodiments, the number of hidden units is somewhere in the range of 5 to 100, with the number increasing with the number of inputs and number of training cases.

One general approach to determining the number of hidden units to use is to apply a regularization approach. In the regularization approach, a new criterion function is constructed that depends not only on the classical training error, but also on classifier complexity. Specifically, the new criterion function penalizes highly complex classifiers; searching for the minimum in this criterion is to balance error on the training set with error on the training set plus a regularization term, which expresses constraints or desirable properties of solutions:

J=Jₚₐₜ+λJ_{reg}.

The parameter λ is adjusted to impose the regularization more or less strongly. In other words, larger values for λ will tend to shrink weights towards zero: typically cross-validation with a validation set is used to estimate λ. This validation set can be obtained by setting aside a random subset of the training population. Other forms of penalty have been proposed, for example the weight elimination penalty (see, e.g., Hastie et al., 2001, The Elements of Statistical Learning, Springer-Verlag, New York).

Another approach to determine the number of hidden units to use is to eliminate—prune—weights that are least needed. In one approach, the weights with the smallest magnitude are eliminated (set to zero). Such magnitude-based pruning can work, but is nonoptimal; sometimes weights with small magnitudes are important for learning and training data. In some embodiments, rather than using a magnitude-based pruning approach, Wald statistics are computed. The fundamental idea in Wald Statistics is that they can be used to estimate the importance of a hidden unit (weight) in a classifier. Then, hidden units having the least importance are eliminated (by setting their input and output weights to zero). Two algorithms in this regard are the Optimal Brain Damage (OBD) and the Optimal Brain Surgeon (OBS) algorithms that use second-order approximation to predict how the training error depends upon a weight, and eliminate the weight that leads to the smallest increase in training error.

Optimal Brain Damage and Optimal Brain Surgeon share the same basic approach of training a network to local minimum error at weight w, and then pruning a weight that leads to the smallest increase in the training error. The predicted functional increase in the error for a change in full weight vector δw is:

$$δ J = {( \frac{\partial J}{\partial w} )}^{t} \cdot δ w + \frac{1}{2} δ {w}^{t} \cdot \frac{{\partial }^{2} J}{\partial {w}^{2}} \cdot δ w + O ( {‖ δ w ‖}^{3} )$$

where

$$\frac{{\partial }^{2} J}{\partial {w}^{2}}$$

is the Hessian matrix. The first term vanishes at a local minimum in error; third and higher order terms are ignored. The general solution for minimizing this function given the constraint of deleting one weight is:

$$δ w = - \frac{{w}_{q}}{{[ {H}^{- 1} ]}_{qq}} {H}^{- 1} \cdot {u}_{q} and {L}_{q} = \frac{1}{2} - \frac{{w}_{q}^{2}}{{[ {H}^{- 1} ]}_{qq}}$$

Here, u_{q} is the unit vector along the qth direction in weight space and L_{q} is approximation to the saliency of the weight q—the increase in training error if weight q is pruned and the other weights updated δw. These equations require the inverse of H. One method to calculate this inverse matrix is to start with a small value, H₀⁻¹=α⁻¹I, where α is a small parameter—effectively a weight constant. Next the matrix is updated with each pattern according to

$${H}_{m + 1}^{- 1} = {H}_{m}^{- 1} - \frac{{H}_{m}^{- 1} {X}_{m + 1} {X}_{m + 1}^{T} {H}_{m}^{- 1}}{\frac{n}{{a}_{m}} + {X}_{m + 1}^{T} {H}_{m}^{- 1} {X}_{m + 1}} \qquad Eqn . 1$$

where the subscripts correspond to the pattern being presented and aₘ decreases with m. After the full training set has been presented, the inverse Hessian matrix is given by H⁻¹=Hₙ⁻¹. In algorithmic form, the Optimal Brain Surgeon method is:

```
  begin initialize n_{H}, w, θ
      train a reasonably large network to minimum error
      do compute H⁻¹ by Eqn. 1
        $\begin{matrix}{q}^{*} ← arg {min}_{q} {w}_{q}^{2} / ( {2 [ {H}^{- 1} ]}_{qq} ) ( saliency {L}_{q} ) \\ w ← w - \frac{{w}_{{q}^{*}}}{{[ {H}^{- 1} ]}_{{q}^{*} {q}^{*}}} {H}^{- 1} {e}_{{q}^{*}} ( saliency {L}_{q} )\end{matrix}$
      until J(w) > θ
    return w
  end
```

The Optimal Brain Damage method is computationally simpler because the calculation of the inverse Hessian matrix in line 3 is particularly simple for a diagonal matrix. The above algorithm terminates when the error is greater than a criterion initialized to be θ. Another approach is to change line 6 to terminate when the change in J(w) due to elimination of a weight is greater than some criterion value. In some embodiments, the back-propagation neural network See, for example Abdi, 1994, “A neural network primer,” J. Biol System. 2, 247-283.

### 5.5.3 Clustering

In some embodiments, features for select biomarkers of the present invention are used to cluster a training set. For example, consider the case in which ten features (corresponding to ten biomarkers) described in the present invention is used. Each member m of the training population will have feature values (e.g. expression values) for each of the ten biomarkers. Such values from a member m in the training population define the vector:

### X₁ₘ X₂ₘ X₃ₘ X₄ₘ X₅ₘ X₆ₘ X₇ₘ X₈ₘ X₉ₘ X₁₀ₘ

where Xᵢₘ is the expression level of the i^{th} biomarker in organism m. If there are m organisms in the training set, selection of i biomarkers will define m vectors. Note that the methods of the present invention do not require that each the expression value of every single biomarker used in the vectors be represented in every single vector m. In other words, data from a subject in which one of the i^{th} biomarkers is not found can still be used for clustering. In such instances, the missing expression value is assigned either a “zero” or some other normalized value. In some embodiments, prior to clustering, the feature values are normalized to have a mean value of zero and unit variance.

Those members of the training population that exhibit similar expression patterns across the training group will tend to cluster together. A particular combination of genes of the present invention is considered to be a good classifier in this aspect of the invention when the vectors cluster into the trait groups found in the training population. For instance, if the training population includes class a: subjects that do not have an affective disorder under study, and class b: subjects that have the affective order under study, an ideal clustering classifier will cluster the population into two groups, with one cluster group uniquely representing class a and the other cluster group uniquely representing class b.

Clustering is described on pages 211-256 of Duda and Hart, Pattern Classification and Scene Analysis, 1973, John Wiley & Sons, Inc., New York, (hereinafter “Duda 1973”). As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda 1973.

Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

### 5.5.4 Support Vector Machines

In some embodiments of the present invention, support vector machines (SVMs) are used to classify subjects using feature values of the genes described in the present invention. SVMs are a relatively new type of learning algorithm. See, for example, Cristianini and Shawe-Taylor, 2000, An Introduction to Support Vector Machines, Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y., Duda, Pattern Classification, Second Edition, 2001, John Wiley & Sons, Inc.; and Hastie, 2001, The Elements of Statistical Learning, Springer, New York; and Furey et al., 2000, Bioinformatics 16, 906-914. When used for classification, SVMs separate a given set of binary labeled data training data with a hyper-plane that is maximally distance from them. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In one approach, when a SVM is used, the feature data is standardized to have mean zero and unit variance and the members of a training population are randomly divided into a training set and a test set. For example, in one embodiment, two thirds of the members of the training population are placed in the training set and one third of the members of the training population are placed in the test set. The expression values for a combination of genes described in the present invention is used to train the SVM. Then the ability for the trained SVM to correctly classify members in the test set is determined. In some embodiments, this computation is performed several times for a given combination of molecular markers. In each iteration of the computation, the members of the training population are randomly assigned to the training set and the test set. Then, the quality of the combination of biomarkers is taken as the average of each such iteration of the SVM computation.

### 5.5.5. Relevance Vector Machines and Genetic Algorithms

A Relevance Vector Machine (RVM) is a kernel based Bayesian statistical model usable in regression as well as supervised multi-class classification problems (Tipping, M: Sparse Bayesian Learning and the Relevance Vector Machine, Journal of Machine Learning Research 1, 2001, 211-244). Used as a classification tool, the trained RVM makes probabilistic predictions regarding the class membership of new data points. In the RVM model it is assumed that a predefined set of explanatory variables (i.e. genes or biomarkers) affects the class membership probability through a logistic link function. To determine the optimum set of explanatory variables selected from a number of candidate variables, the RVM model is operating inside a Genetic optimization algorithm (Deb, K: Multi-Objective Optimization using Evolutionary Algorithms, Wiley, 2001), which evaluates a large number of RVMs that are trained and tested on different subsets of candidate variables. The performance of each variable subset is evaluated through cross validation.

### 5.5.6 Other Data Analysis Algorithms

The data analysis algorithms described above are merely examples of the types of methods that can be used to construct a decision rule for discriminating converters from nonconverters. Moreover, combinations of the techniques described above can be used. Some combinations, such as the use of the combination of decision trees and boosting, have been described. However, many other combinations are possible. In addition, in other techniques in the art such as Projection Pursuit and Weighted Voting can be used to construct decision rules.

### 5.6 Biomarkers

In a particular embodiment, the biomarker profile comprises at least two different biomarkers listed in Table 1A. The biomarker profile further comprises a respective corresponding feature for the at least two biomarkers. Such biomarkers can be, for example, mRNA transcripts, cDNA or some other nucleic acid, for example amplified nucleic acid, or proteins. Generally, the at least two biomarkers are derived from at least two different genes. In the case where a biomarker in the at least two different biomarkers is listed in Table 1A, the biomarker can be, for example, a transcript made by the listed gene, a complement thereof, or a discriminating fragment or complement thereof, or a cDNA thereof, or a discriminating fragment of the cDNA, or a discriminating amplified nucleic acid molecule corresponding to all or a portion of the transcript or its complement, or a protein encoded by the gene, or a discriminating fragment of the protein, or an indication of any of the above. In accordance with such embodiments, the biomarker profiles of the present invention can be obtained using any standard assay known to those skilled in the art, or in an assay described herein, to detect a biomarker. Such assays are capable, for example, of detecting the products of expression (e.g., nucleic acids and/or proteins) of a particular gene or allele of a gene of interest (e.g., a gene disclosed in Table 1A). In one embodiment, such an assay utilizes a nucleic acid microarray.

In some embodiments the biomarker profile has between 2 and 29 biomarkers listed in Table 1A. In some embodiments, the biomarker profile has between 3 and 20 biomarkers listed in Table 1A. In some embodiments, the biomarker profile has between 4 and 15 biomarkers listed in Table 1A. In some embodiments, the biomarker profile has at least 2 biomarkers listed in Table 1A. In some embodiments, the biomarker profile has at least 3 biomarkers listed in Table 1A. In some embodiments, the biomarker profile has at least 4 biomarkers listed in Table 1A. In some embodiments, the biomarker profile has at least 2, 3, 4, 5, 6, 7, 8, 9, 10, 11, 12, 13, 14, 15, 16, 17, 18, 19, 20, 25 or more biomarkers listed in Table 1A. In some embodiments, each such biomarker is a nucleic acid. In some embodiments, each such biomarker is a protein. In some embodiments, some of the biomarkers in the biomarker profile are nucleic acids and some of the biomarkers in the biomarker profile are proteins.

### 5.7 Specific Embodiments

One aspect of the present invention relates to methods of identifying the gene transcription profiles of subjects likely to exhibit symptoms of affective disorders. Such gene transcription profiles are based on transcription analysis of selected genes from biological samples of the subjects, such genes selected from Table 1A.

Using the present invention, it is possible to identify and analyze abundance (e.g. expression levels) of individual biomarkers that may be aggregated into a single profile. Such abundance profiles are used as signatures for disease classification. As discussed below, transcriptional analysis was done to determine the gene expression profile in whole blood samples of control subjects and diseased subjects. Abundance of genes selected from Table 1A is exemplified in Table 4, Table 5, and Table 6. Each of Table 4, Table 5, and Table 6 are representative examples of a gene transcription profile for depressed subjects, severely depressed subjects, and bipolar subjects, respectively, as compared to controls. In one embodiment, a subject having the depression gene transcription profile as shown in Table 4 is diagnosed as having depression. In another embodiment, a subject having the severe depression gene transcription profile as shown in Table 5 is diagnosed as having severe depression. In another embodiment, a subject having the bipolar gene transcription profile as shown in Table 6 is diagnosed as having a bipolar disorder. Further representative examples of a gene transcription profile are shown in Tables 4A and 5B.

In one example, the biomarkers used to determine a gene expression profile were selected from the genes described in Table 1A. Representative transcriptional biomarker probe sets are also described in Table 1A. The probe sets were used to perform quantitative PCR (qPCR) by well-known methods.

An aspect of the invention provides a transcription profile for each subject as determined by transcriptional analysis of genes selected from Table 1A.

Transcriptional analysis can be performed by methods well-known in the art. By way of example, RNA, including messenger RNA (mRNA) may be isolated from cellular material, or fluids containing cellular material, of the animal body, particularly a human body. It is understood that the cellular material contains the cellular contents including mRNA. Biological samples used in the invention may be selected, for example, from peripheral tissues, whole blood, cerebrospinal fluid, peritoneal fluid, and interstitial fluid.

In other embodiments of the invention, the biological sample is selected from the group consisting of whole blood, cerebrospinal fluid, and peripheral tissues. The invention may also be performed using fractions of whole blood selected from the group consisting of red blood cells (RBCs), white blood cells and platelets. White blood cells (leukocytes) include, but are not limited to: neutrophils, basophils, eosinophils, lymphocytes, macrophages and monocytes.

To measure gene expression in a sample, RNA or mRNA in that sample may be subjected to reverse transcription to create copy DNA, and then analyzed by standard methods using probes, or primer sequences, based on the DNA sequence. Each individual gene may be analyzed by polymerase chain reaction (PCR), quantitative PCR, in situ hybridization, Northern blot analysis, solid-support immobilization assays, such as bead-based assays or gene arrays, and other methods well-known in the art.

In accordance with an aspect of the present invention described herein, quantitative PCR (qPCR) is used to measure mRNA levels. One or more nucleic acid probes were used to measure mRNA levels from biological samples. Probes, or primers, are nucleotide (nt) sequences complementary to the genes of interest, and selection and synthesis of such probes/primers is done by methods well known to the skilled artisan. Probes/primers of the present invention are not limited to the nucleotide sequences described in Table 1A.

This invention further provides a method of classification of diseased subjects as compared to control subjects by determining the transcription profile of such subject as analyzed from a biological sample obtained from the subject.

The invention provides a distinctive transcription profile determined by transcriptional analysis of genes selected from Table 1A. Such transcription profile is determined to be distinct in a subject if it is determined to be similar to the transcription profile of known healthy control subjects or known diseased subjects. Similarity to a transcription profile of known healthy control subjects or known diseased subjects is determined by classification methods, such as classification algorithms, as described herein.

In some embodiments, transcription data is collected from a plurality of control subjects as described herein. Transcription data is collected from a plurality of subjects suffering from a disease or disorder, such as an affective disorder, as described herein. Data analysis algorithms are used with each set of transcription data as input in order to discriminate or distinguish the classifying genes contained in each transcription data set. Such algorithm is typically described as a classification algorithm, also known as a “classifier”. Data analysis algorithms used to perform this task are well known to those skilled in the art and the following examples may be used: Random Forest (Breiman, L., 2001, Machine Learning 45(1):5-32), Support Vector Machine (SVM) (Cortes, C. and Vapnik, V. 1995, Machine Learning, 20(3):273-97), Stepwise Logistic Regression (SLR) (Ersbøll, B. K. and Conradsen, K. (2005) An Introduction to Statistics. 7th ed. IMM; Draper, N. and Smith, H. (1981) Applied Regression Analysis, 2d Edition, New York: John Wiley & Sons, Inc.), recursive partitioning (RPART) (James K. E. et al, 2005, Statistics in Medicine, 24 (19): 3019-35), Penalized Logistic Regression Analysis (PELORA) (Dettling, M., 2003, Proceedings of the 3^{rd} International Workshop on Distributed Statistical Computing, March 20-22, Vienna Austria, Hornick, Leisch and Seilis, eds.), Neural Networks, Relevance Vector Machines (RVM), LogitBoost (Friedman, J., Hastie, T. and Tibshirani, R. 2000, Annals of Statistics 28(2):337-407), Prediction Analysis of Microarrays (PAM), and others (see V. N. Vapnik, Statistical Learning Theory, Wiley, New York, 1998). Such classification algorithms, or “classifiers”, are tuned and trained to provide output regarding the classification of patients based on their transcription data.

Classifying genes or biomarkers selected by the trained classification algorithm yield a predictive measure of the transcription data associated with the class to which a particular data set belongs, e.g. either the class related to control data or the class related to disease data.

While not wishing to be bound by any particular theory, the Random Forest algorithm is considered an ensemble learning method, which classifies objects based on the outputs from a large number of decision trees. Each decision tree is trained on a bootstrap sample of the available data, and each node in the decision tree is split by the best explanatory variables (i.e. genes or biomarkers). Random Forest can both provide automatic variable selection and describe non-linear interactions between the selected variables.

Stepwise Logistic Regression (SLR) is considered a statistical model which predicts the probability of occurrence of an event by fitting the data input to a logistic curve. In the logistic model it is assumed that a predefined set of explanatory variables (i.e. genes or biomarkers) affects the probability through a logistic link function. To determine the optimum set of explanatory variables selected from a number of candidate variables, a large number of logistic regression models are built from an initial model in a stepwise fashion and compared through the evaluation of Akaike Information Criteria (AIC) in order to determine the most accurate model (Burnham, K. P., and D. R. Anderson, 2002. Model Selection and Multimodel Inference: A Practical-Theoretic Approach, 2nd ed. Springer-Verlag).

Support Vector Machines (SVMs) are considered to belong to a family of generalized linear classifiers. Viewing the input data in 2-group classification as two sets of vectors in an n-dimensional space, an SVM separates the data by the hyperplane, which maximizes the margin between the two sets of vectors. The vectors, which take the minimum distance to the maximizing hyperplane, are called support vectors. SVM does not provide automatic variable (i.e. gene or biomarker) selection.

Relevance Vector Machines (RVMs) assume that a predefined set of explanatory variables (i.e. genes or biomarkers) affects the class membership probability through a logistic link function. RVMs seek to determine the optimum set of explanatory variables selected from a number of candidate variables. The RVM may operate with a Genetic optimization algorithm which evaluates and cross-validates many RVMs and selects the optimum set of candidate variables (i.e. genes or biomarkers).

Transcription profiles built with a classification algorithm are further trained using one of the aforementioned data analysis algorithms. Classification error is a measure of accuracy for which the trained classification algorithm predicts membership within a class. Classification error may be determined by cross-validation methods such as leave-one-out cross validation (LOOCV), K-fold validation, or ten-fold validation (Devijver, P. A., and J. Kittler, 1982, Pattern Recognition: A Statistical Approach, Prentice-Hall, London). Accuracy of the algorithm with a prescribed transcription profile may be measured by determining the number of true positives (TP), true negatives (TN), false positives (FP), and false negatives (FN) that were predicted by that algorithm during training. Accuracy is measured as:

Accuracy=(TP+TN)/TP+TN+FP+FN)

Positive Predictive Value (PPV), or the percentage of diseased subjects that have been scored positively by the algorithm is measured as:

PPV=TP/TP+FP

Negative Predictive Value (NPV), or the percentage of control subjects (that do not have the disease) and have been scored negatively by the algorithm is measured as:

NPV=TN/TN+FN

The performance of a classification algorithm is also determined by a Jaccard similarity coefficient (Jaccard Index), which assesses how well the classification has identified the correct variables (i.e. genes). Accuracy of a trained classification algorithm can be greater than about 60%, 65%, 70%, 75%, 80%, 85%, 90%, or 95%. Jaccard Index of a trained classification algorithm can be greater than about 60%, 65%, 70%, 75%, 80%, 85%, 90%, or 95%. PPV and NPV of a trained classification algorithm can be greater than about 60%, 65%, 70%, 75%, 80%, 85%, 90%, or 95%.

Classification of subjects may be useful for the diagnosis of a subject having an affective disorder or likely to exhibit the symptoms of an affective disorder. Gene transcription profiles for classification of subjects are based on the transcription analysis of genes in Table 1A. The transcription profile of a subject as analyzed by the methods described herein will be indicative of whether or not the subject belongs to the class of diseased subjects

In some embodiments, the present invention provides a method of diagnosing an affective disorder in a test subject, the method comprising evaluating whether a plurality of features of a plurality of biomarkers in a biomarker profile of the test subject satisfies a value set, wherein satisfying the value set predicts that the test subject has said affective disorder, and wherein the plurality of features are measurable aspects of the plurality of biomarkers, the plurality of biomarkers comprising at least two biomarkers listed in Table 1A. The method further comprises outputting a diagnosis of whether the test subject has the affective disorder to a user interface device, a monitor, a tangible computer readable storage medium, or a local or remote computer system; or displaying a diagnosis of whether the test subject has the affective disorder in user readable form.

In some embodiments of the invention, the plurality of biomarkers consists of between 2 and 29 biomarkers listed in Table 1A. In other embodiments, the plurality of biomarkers consists of between 3 and 20 biomarkers listed in Table 1A. In still other embodiments, the plurality of biomarkers comprises at least two, three, four or five biomarkers listed in Table 1A.

In some embodiments, the plurality of features consists of between 2 and 29 features corresponding to between 2 and 29 biomarkers listed in Table 1A. In other embodiments, the plurality of features consists of between 3 and 15 features corresponding to between 3 and 15 biomarkers listed in Table 1A. In still other embodiments, the plurality of features comprises at least 2 features corresponding to at least 2 biomarkers listed in Table 1A.

In other embodiments, the plurality of biomarkers comprises ERK1 and MAPK14. In other embodiments, the plurality of biomarkers comprises Gi2 and IL-1b. In other embodiments, the plurality of biomarkers comprises ARRB1 and MAPK14. In other embodiments, the plurality of biomarkers comprises ERK1 and IL1b.

In some aspects of the invention, each biomarker in said plurality of biomarkers is a nucleic acid. In other aspects, each biomarker is in said plurality of biomarkers is a DNA, a cDNA, an amplified DNA, an RNA, or an mRNA. In still other aspects, each biomarker in said plurality of biomarkers is a protein.

In other embodiments, a feature in said plurality of features in the biomarker profile of the test subject is a measurable aspect of a biomarker in the plurality of biomarkers and a feature value for said feature is determined using a biological sample taken from said test subject. In other embodiments, the feature is abundance of said biomarker in the biological sample. In still other embodiments, the biological sample is a peripheral tissue, whole blood, a cerebrospinal fluid, a peritoneal fluid, an interstitial fluid, red blood cells, white blood cells, or platelets.

In another embodiment, the feature in said plurality of features is a measurable aspect of a biomarker in said biomarker profile and a feature value for said feature is determined using a sample taken from said test subject. In some embodiments, a biomarker in the biomarker profile is an indication of a nucleic acid or an indication of a protein. In other embodiments, a biomarker in the biomarker profile is an indication of an mRNA molecule or an indication of a cDNA molecule. In some embodiments, the indication of an mRNA molecule or cDNA molecule is a transcript value such as copies per ng of cDNA. In other embodiments, a first biomarker in the biomarker profile is an indication of a nucleic acid and a second biomarker in the biomarker profile is an indication of a protein.

In some aspects of the invention, the value set comprises abundance of biomarkers as set forth in Table 4, and satisfying the value set of Table 4 predicts that the subject has depression. In other aspects, the value set comprises abundance of biomarkers as set forth in Table 5, and satisfying the value set of Table 5 predicts that the subject has severe depression. In other aspects, the value set comprises abundance of biomarkers as set forth in Table 6, and satisfying the value set of Table 6 predicts that the subject has bipolar depression. Further, the present invention provides value sets for a diagnosis of depression as in Table 4A and value sets for a diagnosis of severe depression as in Table 5B.

The value sets depicted in Tables 4, 5 and 6 are represented by abundance of biomarkers in copies per ng of cDNA, i.e. transcript of the biomarker gene. For example, the range of transcript values for a depressed subject for the biomarker ARRB1 in Table 4 is 189062±62727 copies/ng cDNA, which is equivalent to a range of 126335 to 251789 copies/ng cDNA. The range of transcript values for a depressed subject for the biomarker CD8a in Table 4 is 8304±5825 copies/ng cDNA, which is equivalent to a range of 2479 to 14129 copies/ng cDNA. In some aspects of the invention, satisfying the value set means having values within the given range for each biomarker.

In some embodiments, the value set comprising abundance of ERK1 within the range of 15148 to 35504 copies per ng of cDNA and abundance of MAPK14 within the range 39241 to 107071 copies per ng of cDNA predicts that the subject has depression. In other embodiments, the value set comprising abundance of Gi2 within the range of 61734 to 168500 copies per ng of cDNA and abundance of IL1b within the range 15939 to 43323 copies per ng of cDNA predicts that the subject has depression. In other embodiments, the value set comprising abundance of ARRB1 within the range of 126335 to 251789 copies per ng of cDNA and abundance of MAPK14 within the range 39241 to 107071 copies per ng of cDNA, predicts that the subject has depression. In other embodiments, the value set comprising abundance of ERK1 within the range of 15148 to 35504 copies per ng of cDNA and abundance of IL1b within the range 15939 to 43323 copies per ng of cDNA predicts that the subject has depression.

In other embodiments, the value set comprising a ratio of abundance of ERK1 divided by abundance of MAPK14 within the range 0.25 to 0.45 predicts that the subject has depression. In other embodiments, the value set comprising a ratio of abundance of Gi2 divided by abundance of IL1b within the range 0.16 to 0.36 predicts that the subject has depression. In other embodiments, the value set comprising a ratio of abundance of MAPK14 divided by abundance of ARRB1 within the range 0.29 to 0.49 predicts that the subject has depression. In other embodiments, the value set comprising a ratio of abundance of ERK1 divided by abundance of IL1b within the range 0.0.75 to 0.95 predicts that the subject has depression.

In other embodiments, the value set comprising a ratio of abundance of ERK1 divided by abundance of MAPK14 within the range 0.19 to 0.39 predicts that the subject has severe depression. In other embodiments, the value set comprising a ratio of abundance of Gi2 divided by abundance of IL1b within the range 0.18 to 0.38 predicts that the subject has severe depression. In other embodiments, the value set comprising a ratio of abundance of MAPK14 divided by abundance of ARRB1 within the range 0.32 to 0.52 predicts that the subject has severe depression. In other embodiments, the value set comprising a ratio of abundance of ERK1 divided by abundance of IL1b within the range 0.60 to 0.80 predicts that the subject has severe depression.

In other aspects of the above method, the method further comprises constructing, prior to the evaluating step, said biomarker profile. In other embodiments, the constructing step comprises' obtaining said plurality of features from a biological sample of said test subject. In some aspects, the biomarker profile is constructed by determining the ratio of abundance of biomarkers by dividing the feature value of a first biomarker by the feature value of a second biomarker. Such biomarker profile may be constructed using the values shown in Table 4, Table 5 or Table 6.

In other embodiments, the sample is a peripheral tissue, whole blood, a cerebrospinal fluid, a peritoneal fluid, an interstitial fluid, red blood cells, white blood cells, or platelets.

In still other aspects of the above method, the method further comprises constructing, prior to the evaluating step, said first value set. In other embodiments, the constructing step comprises applying a data analysis algorithm to features obtained from members of a population.

In some aspects, the features are measurable aspects of biomarkers comprising ERK1 and MAPK14, and feature values are determined using a blood sample taken from said test subject

In other embodiments, the population comprises a first plurality of biological samples from a first plurality of control subjects not having the affective disorder and a second plurality of biological samples from a second plurality of subjects having the affective disorder. In still other embodiments, the data analysis algorithm is a decision tree, predictive analysis of microarrays, a multiple additive regression tree, a neural network, a clustering algorithm, principal component analysis, a nearest neighbor analysis, a linear discriminant analysis; a quadratic discriminant analysis, a support vector machine, an evolutionary method, a relevance vector machine, a genetic algorithm, a projection pursuit, or weighted voting.

In another embodiment, the constructing step generates a decision rule and wherein said evaluating step comprises applying said decision rule to the plurality of features in order to determine whether they satisfy the first value set. In some embodiments, the decision rule classifies subjects in said population as (1) subjects that do not have the affective disorder and (ii) subjects that do have the affective disorder with an accuracy of seventy percent or greater. In other embodiments, the decision rule classifies subjects in said population as (i) subjects that do not have the affective disorder and (ii) subjects that do have the affective disorder with an accuracy of ninety percent or greater.

In certain aspects of the invention, the affective disorder is bipolar disorder I, bipolar disorder II, a dysthymic disorder, or a depressive disorder. In other aspects, the affective disorder is mild depression, moderate depression, severe depression, atypical depression, melancholic depression, or a borderline personality disorder. In still other aspects, the affective disorder is (i) post traumatic stress disorder or (ii) trauma without post traumatic stress disorder. In some aspects, the affective disorder is acute post traumatic stress disorder or remitted post traumatic stress disorder.

The present invention provides a kit used for diagnosing an affective disorder in a test subject, the kit comprising reagents and instructions for evaluating whether a plurality of features of a plurality of biomarkers in a biomarker profile of the test subject satisfies a value set, wherein satisfying the value set predicts that the test subject has said affective disorder, and wherein the plurality of features are measurable aspects of the plurality of biomarkers, the plurality of biomarkers comprising at least two biomarkers listed in Table 1A. In some aspects, the reagents comprise probes and/or primers that recognize nucleotide sequences of the biomarkers selected from Table 1A. The kits of the invention are used to generate biomarker profiles according to the invention. In some aspects, the kits of the invention provide instructions for testing and evaluating the biomarker profile of the test subject from a plurality of biomarkers comprising at least two biomarkers listed in Table 1A. In other aspects, the kits of the invention provide instructions containing value sets in order to determine if the biomarker profile of the test subject satisfies such value set.

The present invention also provides a computer program product, wherein the computer program product comprises a computer readable storage medium and a computer program mechanism embedded therein, the computer program mechanism comprising instructions for carrying out any of the above methods. In some embodiments, the computer program mechanism further comprises instructions for outputting a diagnosis of whether the test subject has the affective disorder to a user interface device, a monitor, a tangible computer readable storage medium, or a local or remote computer system; or displaying a diagnosis of whether the test subject has the affective disorder in user readable form.

The present invention also provides a computer comprising: one or more processors; a memory coupled to the one or more processors, the memory storing instructions for carrying out any of the above methods. In some aspects of the invention, the memory further comprises instructions for outputting a diagnosis of whether the test subject has the affective disorder to a user interface device, a monitor, a tangible computer readable storage medium, or a local or remote computer system; or displaying a diagnosis of whether the test subject has the affective disorder in user readable form.

The present invention further provides a method of determining a likelihood that a test subject exhibits a symptom of an affective disorder, the method comprising: evaluating whether a plurality of features of a plurality of biomarkers in a biomarker profile of the test subject satisfies a value set, wherein satisfying the value set provides said likelihood that the test subject exhibits a symptom of an affective disorder, and wherein the plurality of features are measurable aspects of the plurality of biomarkers, the plurality of biomarkers comprising at least two biomarkers listed in Table 1A.

In some embodiments, the plurality of biomarkers comprises ERK1 and MAPK14. In other embodiments, the plurality of biomarkers comprises Gi2 and IL-1b. In other embodiments, the plurality of biomarkers comprises ARRB1 and MAPK14. In other embodiments, the plurality of biomarkers comprises ERK1 and IL1b.

In some embodiments of the invention, the plurality of biomarkers comprises ERK1, PBR and MAPK14. In another embodiment, the plurality of biomarkers comprises PBR, Gi2 and IL 1b. In other embodiments, the plurality of biomarkers comprises ERK1, ARRB1 and MAPK14. In some embodiments, the plurality of biomarkers comprises MAPK14, ERK1 and CD8b. In other embodiments, the plurality of biomarkers comprises MAPK14, ERK1 and P2X7. In still other embodiments, the plurality of biomarkers comprises ARRB1, IL6 and CD8a. In certain embodiments, the plurality of biomarkers comprises ARRB1, ODC1 and P2X7.

In still other embodiments, the method further comprises outputting the likelihood that the test subject exhibits a symptom of an affective disorder to a user interface device, a monitor, a tangible computer readable storage medium, or a local or remote computer system; or displaying the likelihood that the test subject exhibits a symptom of an affective disorder in user readable form.

The present invention provides a transcription profile which is a measure of transcriptional analysis for each biological sample collected from a plurality of control subjects. The present invention provides a transcription profile which is a measure of transcriptional analysis for each biological sample collected from a plurality of depressed subjects, severely depressed subjects, or bipolar subjects. The present invention further provides a transcription profile which is a measure of transcriptional analysis for each biological sample collected from a plurality of borderline personality disorder subjects. The present invention provides a transcription profile which is a measure of transcriptional analysis for each biological sample collected from a plurality of PTSD subjects.

The invention also provides that a transcription profile comprising the collective measure of a first plurality of control subjects is stored, for example in a database. A transcription profile comprising the collective measure of a second plurality of subjects, for example, diseased subjects, is compared to the transcription profile of the first plurality of control subjects using a data analysis algorithm, particularly a trained classification algorithm. The trained classification algorithm classifies each set of subjects. Trained classification algorithms provide predictive values useful for diagnosing and assigning a classification. Trained classification algorithms provide predictive values useful for predicting the likelihood that a subject will exhibit symptoms of a disorder.

Another embodiment of this invention relates to diagnosing or predicting a subject's susceptibility to a disease or disorder or predicting the likelihood of exhibiting symptoms of a disorder based on the distinct transcription profile of the subject as compared to that of healthy control subjects and diseased subjects. Gene transcription profiles for diagnostic uses are based on transcription analysis of genes selected from Table 1A.

One aspect of the present invention relates to diagnosis of different types of affective disorders, particularly major depressive disorder, bipolar disorder, borderline personality disorder, and post-traumatic stress disorder.

Another aspect of the invention relates to differentiating patient populations by identifying transcription profiles. For example, patients that would normally be diagnosed for major depression, may be segmented by transcription profile into subtypes of depression, for example as melancholic and atypical depression. There is evidence for differential treatment response for these subtypes of depression. Patients that exhibit co-morbidity, i.e. meet the DSM-IV® criteria for more than one disorder, will benefit from identification of a transcription profile. Transcription profiles may identify a common biological basis for one disorder.

By way of the above methods, the present invention provides, in one embodiment, a transcription profile which is a measure of transcriptional analysis for biological samples collected from a plurality of healthy control subjects. The present invention also provides a transcription profile which is a measure of transcriptional analysis for biological samples collected from a plurality of affective disorder subjects. For example, the present invention also provides a transcription profile which is a measure of transcriptional analysis for biological samples collected from a plurality of depressed, severely depressed, or bipolar subjects. The present invention provides a transcription profile which is a measure of transcriptional analysis for biological samples collected from a plurality of depressed subjects as in Table 4. The present invention provides a transcription profile which is a measure of transcriptional analysis for biological samples collected from a plurality of severely depressed subjects as in Table 5. The present invention also provides a transcription profile which is a measure of transcriptional analysis for biological samples collected from a plurality of bipolar subjects as in Table 6. The present invention further provides a transcription profile which is a measure of transcriptional analysis for biological samples collected from a plurality of borderline personality disorder subjects. The present invention provides a transcription profile which is a measure of transcriptional analysis for biological samples collected from a plurality of PTSD subjects. In one embodiment of the invention, the biological sample is whole blood.

The invention also provides that a transcription profile comprising the collective measure of a first plurality of control subjects is stored, for example in a database. A transcription profile comprising the collective measure of a second plurality of subjects, for example, diseased subjects, is compared to the transcription profile of the first plurality of control subjects using a classification algorithm. The classification algorithm provides output that classifies each of the subjects.

In some aspects of the invention, the transcription profile is determined from the transcriptional analysis of genes selected from the group consisting of ADA, ARRB1, ARRB2, CD8a, CD8b, CREB1, CREB2, DPP4, ERK1, ERK2, Gi2, Gs, GR, IL1b, IL6, IL8, INDO, MAPK14, MAPK8, MKP1, MR, ODC1, P2X7, PBR, PREP, RGS2, S100A10, SERT and VMAT2.

In another embodiment, the transcription profile is determined from the transcriptional analysis of at least three genes selected from the group consisting of ADA, ARRB1, ARRB2, CD8a, CD8b, CREB1, CREB2, DPP4, ERK1, ERK2, Gi2, Gs, GR, IL 1b, IL6, IL8, INDO, MAPK14, MAPK8, MKP1, MR, ODC1, P2X7, PBR, PREP, RGS2, S100A10, SERT and VMAT2.

In some embodiments, the transcription profile is determined from the transcriptional analysis of genes selected from the group consisting of ARRB1, ARRB2, CD8a, CREB1, CREB2, ERK2, Gi2, MAPK14, ODC1, P2X7, and PBR.

In another embodiment, the transcription profile is determined from the transcriptional analysis of genes selected from the group consisting of CD8a, ERK1, MAPK14, P2X7, and PBR.

In another embodiment, the transcription profile is determined from the transcriptional analysis of genes selected from the group consisting of Gi2, GR, and MAPK14.

In another embodiment, the transcription profile is determined from the transcriptional analysis of genes selected from the group consisting of Gi2, GR, MAPK14, and MR.

In another embodiment, the transcription profile is determined from the transcriptional analysis of genes selected from the group consisting of ARRB1, ARRB2, CD8b, ERK2, IDO, IL-6, MR, ODC1, PREP and RGS2.

In another embodiment, the transcription profile is determined from the transcriptional analysis of genes selected from the group consisting of ARRB1, CREB1, ERK2, Gs, IL-6, MKP1, and RGS2.

In another embodiment, the transcription profile is determined from the transcriptional analysis of genes selected from the group consisting of ERK1 and MAPK14. In another embodiment, the transcription profile is determined from the transcriptional analysis of genes selected from the group consisting of Gi2 and IL1b. In another embodiment, the transcription profile is determined from the transcriptional analysis of genes selected from the group consisting of ARRB1 and MAPK14. In another embodiment, the transcription profile is determined from the transcriptional analysis of genes selected from the group consisting of ERK1 and IL1b.

In another embodiment, the transcription profile is determined from the transcriptional analysis of genes selected from the group consisting of ERK1, MAPK14, and P2X7. In another embodiment, the transcription profile is determined from the transcriptional analysis of genes selected from the group consisting of Gi2, IL1b, and PBR. In another embodiment, the transcription profile is determined from the transcriptional analysis of genes selected from the group consisting of ARRB1, ODC1, and P2X7. In another embodiment, the transcription profile is determined from the transcriptional analysis of genes selected from the group consisting of ARRB1, CD8a, and IL6. In another embodiment, the transcription profile is determined from the transcriptional analysis of genes selected from the group consisting of CD8b, ERK1, and MAPK14. In another embodiment, the transcription profile is determined from the transcriptional analysis of genes selected from the group consisting of ARRB1, ERK1, and MAPK14. In another embodiment, the transcription profile is determined from the transcriptional analysis of genes selected from the group consisting of ERK1, MAPK14, and PBR.

An aspect of the present invention provides a method for diagnosing an affective disorder in a subject comprising identifying a transcription profile in the subject, and, comparing such transcription profile to the profile of a control subject or group of healthy control subjects, thereby diagnosing whether the subject exhibits an affective disorder based on the presence or absence of changes or differences in the transcription profile.

In some embodiments of the invention, the affective disorder is selected from the group consisting of depression, severe depression, bipolar disorder, borderline personality disorder. In some embodiments, the affective disorder is selected from post traumatic stress disorder or trauma without post traumatic stress disorder. In other embodiments, the affective disorder is selected from acute post traumatic stress disorder or remitted post traumatic stress disorder.

One aspect of the invention provides a method for diagnosing whether a subject exhibits an affective disorder comprising:
(a) obtaining a biological sample from a subject suspected of having an affective disorder;
(b) measuring mRNA levels in the biological sample, wherein the mRNA levels are mRNA levels of genes selected from the group consisting of ADA, ARRB1, ARRB2, CD8a, CD8b, CREB1, CREB2, DPP4, ERK1, ERK2, Gi2, Gs, GR, IL1b, IL6, IL8, INDO, MAPK14, MAPK8, MKP1, MR, ODC1, P2X7, PBR, PREP, RGS2, S100A10, SERT and VMAT2;
(c) collecting and storing the mRNA levels as mRNA data in a computer medium;
(d) processing such mRNA data via a classification algorithm, whereby the processing determines whether the mRNA data is the same or different from mRNA data of healthy control subjects; and
(e) providing output data which classifies the subject,
thereby diagnosing whether the subject exhibits an affective disorder.

The present invention further provides methods for predicting a subject's susceptibility to an affective disorder by comparing the subject's transcription profile of genes selected from the group consisting of ADA, ARRB1, ARRB2, CD8a, CD8b, CREB1, CREB2, DPP4, ERK1, ERK2, Gi2, Gs, GR, IL1b, IL6, IL8, INDO, MAPK14, MAPK8, MKP1, MR, ODC1, P2X7, PBR, PREP, RGS2, S100A10, SERT and VMAT2, to the transcription profile of said genes of a plurality of healthy control subjects.

One aspect of the invention provides a method for predicting the likelihood of a subject exhibiting symptoms of an affective disorder comprising:
(a) obtaining a biological sample from a subject;
(b) measuring mRNA levels wherein the mRNA levels are mRNA levels of genes selected from the group consisting of ADA, ARRB1, ARRB2, CD8a, CD8b, CREB1, CREB2, DPP4, ERK1, ERK2, Gi2, Gs, GR, IL1b, IL6, IL8, INDO, MAPK14, MAPK8, MKP1, MR, ODC1, P2X7, PBR, PREP, RGS2, S100A10, SERT and VMAT2;
(c) collecting and storing the mRNA levels as mRNA data in a computer medium;
(d) processing such mRNA data via a classification algorithm, whereby the processing determines whether the mRNA data is the same or different from mRNA data of healthy control subjects; and
(e) providing output data which classifies the subject,
thereby predicting the likelihood of a subject exhibiting symptoms of an affective disorder.

In another embodiment, the methods can comprise measuring mRNA levels of at least two genes selected from the group consisting of ADA, ARRB1, ARRB2, CD8a, CD8b, CREB1, CREB2, DPP4, ERK1, ERK2, Gi2, Gs, GR, IL1b, IL6, IL8, INDO, MAPK14, MAPK8, MKP1, MR, ODC1, P2X7, PBR, PREP, RGS2, S100A10, SERT and VMAT2.

In other embodiments, the methods comprise measuring mRNA levels of any 3, 4, 5, 6, 7, 8, 9, 10, 11, 12, 13, 14, 15, 16, 17, 18, 19, 20, 21, 22, 23, 24, 25, 26, 27, or 28 genes listed in Table 1A.

In other embodiments, the methods comprise measuring mRNA levels of genes selected from the group consisting of ARRB1, ARRB2, CD8a, CREB1, CREB2, ERK2, Gi2, MAPK14, ODC1, P2X7, and PBR.

In another embodiment, the methods comprise measuring mRNA levels of genes selected from the group consisting of CD8a, ERK1, MAPK14, P2X7, and PBR.

In another embodiment, the methods comprise measuring mRNA levels of genes selected from the group consisting of Gi2, GR, and MAPK14.

In another embodiment, the methods comprise measuring mRNA levels of genes selected from the group consisting of Gi2, GR, MAPK14, and MR.

In another embodiment, the methods comprise measuring mRNA levels of genes selected from the group consisting of ARRB1, ARRB2, CD8b, ERK2, IDO, IL-6, MR, ODC1, PREP and RGS2.

In another embodiment, the methods comprise measuring mRNA levels of genes selected from the group consisting of ARRB1, CREB1, ERK2, Gs, IL-6, MKP1, and RGS2.

In another embodiment, the methods comprise measuring mRNA levels of genes selected from the group consisting of ERK1 and MAPK14. In another embodiment, the methods comprise measuring mRNA levels of genes selected from the group consisting of Gi2 and IL1b. In another embodiment, the methods comprise measuring mRNA levels of genes selected from the group consisting of ARRB1 and MAPK14. In another embodiment, the methods comprise measuring mRNA levels of genes selected from the group consisting of ERK1 and IL1b.

In another embodiment, the methods comprise measuring mRNA levels of genes selected from the group consisting of ERK1, MAPK14, and P2X7. In another embodiment, the methods comprise measuring mRNA levels of genes selected from the group consisting of Gi2, IL1b, and PBR. In another embodiment, the methods comprise measuring mRNA levels of genes selected from the group consisting of ARRB1, ODC1, and P2X7. In another embodiment, the methods comprise measuring mRNA levels of genes selected from the group consisting of ARRB1, CD8a, and IL6. In another embodiment, the methods comprise measuring mRNA levels of genes selected from the group consisting of CD8b, ERK1, and MAPK14. In another embodiment, the methods comprise measuring mRNA levels of genes selected from the group consisting of ARRB1, ERK1, and MAPK14. In another embodiment, the methods comprise measuring mRNA levels of genes selected from the group consisting of ERK1, MAPK14, and PBR.

In some embodiments of the invention, the affective disorder is selected from the group consisting of depression, severe depression, bipolar disorder, borderline personality disorder. In some embodiments, the affective disorder is selected from post traumatic stress disorder or trauma without post traumatic stress disorder. In other embodiments, the affective disorder is selected from acute post traumatic stress disorder or remitted post traumatic stress disorder.

In some embodiments, the above methods are computer-assisted methods.

### 5.7 Affective Disorders

The psychiatric or mental disorders described herein, and their clinical manifestations, are known to practicing psychiatrists. The specific symptoms of each disorder can be recognized by most psychiatrists.

The Diagnostic and Statistical Manual of Mental Disorders, Fourth Edition, Text Revision (DSM-IV-TR®), published by the American Psychiatric Association (October 1994, text revision May 2000), is the standard for clinical classification of mental disorders used by physicians in the United States. The symptomatology and diagnostic criteria for mental/psychiatric disorders are set out in the DSM-IV-TR® guidelines.

### 5.7.1 Depressive Disorders

The DSM-IV-TR® lists specific diagnostic criteria for depression and major depressive disorder (MDD).

The DSM-IV-TR® defines a major depressive episode as a syndrome in which, during the same 2-week period, at least five of the following symptoms present and manifest themselves as a change from a previous state of well-functioning (moreover, the symptoms must include either (1) or (2)):

1. Depressed mood
2. Diminished interest or pleasure
3. Significant weight loss or gain
4. Insomnia or hypersomnia
5. Psychomotor agitation or retardation
6. Fatigue or loss of energy
7. Feelings of worthlessness
8. Diminished ability to think or concentrate; indecisiveness
9. Recurrent thoughts of death, suicidal ideation, suicide attempt, or specific plan for suicide

DSM-IV-TR® further includes descriptions of symptoms that must be present in various subtypes of depression. Depression can be noted to be with or without psychotic symptoms and may have melancholic or catatonic features or be classified as an atypical depression.

Depending upon the number and severity of the symptoms exhibited by the patient, a depressive episode may be specified as mild, moderate or severe. Clinicians may also determine whether the patient is suffering from typical (melancholic), atypical, catatonic, or psychotic depression.

Clinically, depression is considered to be a very heterogeneous disease. Gene expression profiles of depressed patients may reflect this heterogeneity. Based on the present invention, it is possible to better define these subtypes of depression based on gene expression profiles, in order to better classify or diagnose patients. Subsequently, the development and administration of drugs can be tailored to patients suffering from subtypes of depression.

By obtaining and analyzing clinical history and symptom information from controls, gene expression profiles are also used to predict the likelihood of a subject exhibiting symptoms of the disorders described herein.

Depressive disorders, bipolar disorders and dysthymic disorders are considered part of the category of mood disorders.

The subject invention provides an objective measure of a transcription profile indicative of a depressive disorder, such as mild, moderate, or severe depression. The subject invention also provides transcription profiles for the classification of subtypes of depressive disorders. The invention further provides methods for diagnosing a subject with a depressive disorder, such as mild, moderate, or severe depression.

### 5.7.2 Bipolar Disorder

As described for depression, bipolar disorder (BD) is a heterogeneous disease and is divided into subcategories or subtypes, including bipolar I, bipolar II and cyclothymia. Bipolar disorder, also known as manic-depressive illness, is a brain disorder that causes unusual shifts in a person's mood, energy, and ability to function. Different from the normal “ups and downs” that all individuals experience, the symptoms of bipolar disorder are severe, and can result in damaged relationships, poor job or school performance, and even suicide.

BD manifests as intermittent episodes of mania and depression typically recurring across one's life span. Between episodes, most people with bipolar disorder are free of symptoms, or may have some residual symptoms. Depressive episodes are often present, and may be major or severe. Manic episodes are characterized by symptoms such as profound mood disturbances which are sufficient to cause impairment at work or danger to the patient or others, and are not the result of substance abuse or a medical condition, diminished need for sleep, excessive talking or pressured speech, and/or racing thoughts or flight of ideas, and more, as described according to the DSM-IV-TR®.

The present invention provides methods for diagnosing a subject with bipolar disorder. BD patients would benefit from an objective measure of transcription profiles indicative of bipolar disorder.

### 5.7.3 Borderline Personality Disorder

Borderline personality disorder (BPD) comprises a pattern of instability of self-image, interpersonal relationships and affects, with marked impulsivity. This instability often disrupts family and work life and an individual's self-identity.

The DSM-IV-TR® characterizes BPD as indicated by at least five of the following:

1. A pattern of unstable and intense interpersonal relationships characterized by alternating between extremes of over-idealization and devaluation.
2. Impulsivity in at least two areas that are potentially self-damaging, e.g., spending, sex, substance use, shoplifting, reckless driving, or binge eating.
3. Affective instability due to marked reactivity of mood.
4. Inappropriate, intense anger or lack of control of anger, e.g., frequent displays of temper, constant anger or recurrent physical fights.
5. Recurrent suicidal threats, gestures, or behavior or self-mutilating behavior.
6. Identity disturbance; marked and persistent unstable self-image.
7. Chronic feelings of emptiness or boredom.
8. Frantic efforts to avoid real or imagined abandonment.
9. Transient, stress-related paranoid ideation or severe dissociative symptoms.

Patients with BPD are among the most challenging and treatment-resistant patients seen in psychotherapy.

The present invention provides methods for diagnosing a subject with BPD. BPD patients would benefit from an objective measure of transcription profiles indicative of borderline personality disorder.

### 5.7.4 Post Traumatic Stress Disorder (PTSD)

The DSM-IV-TR® describes Post Traumatic Stress Disorder as the development of characteristic symptoms following exposure to an extreme traumatic stressor, involving direct personal experience of an event that involves actual or threatened death or serious injury. The person may have witnessed an event that involves death, injury, or a threat to physical integrity of another person. The person's response to the event involves intense fear, helplessness or horror. The person may have persistent recollections of the event, including images, thoughts, or perceptions, or may have recurrent distressing dreams of the event.

The present invention provides methods for diagnosing a subject with acute PTSD, remitted PTSD, or trauma without PTSD. Patients/subjects would benefit from an objective measure of transcription profiles indicative of acute PTSD, remitted PTSD, or trauma without PTSD.

It is possible to determine, differentiate, and/or distinguish between normal, or healthy, subjects and subjects suffering from affective disorders based on the transcription profiles identified by the above described methods. By way of example, the invention will be better understood by the experimental details that follow. One skilled in the art will readily appreciate that the specific methods and results discussed therein are merely illustrative of the invention as described more fully in the claims which follow thereafter.

### 6 EXPERIMENTAL DETAILS

Total RNA isolation. Human blood was collected into PAXgene™ blood RNA tubes (PreAnalytiX, Hombrechtikon, CH), mixed by inversion several times and stored at −20° or −80° C. until processing for RNA isolation. Processing was begun by incubating the samples at room temperature overnight followed by centrifugation at 3000×G for 10 minutes. The supernatant was decanted and the pellet resuspended in 5 ml water, followed by another centrifugation step. The washing and centrifugation steps were repeated a second time and the pellet was resuspended in the residual water remaining in the tube (about 100 ul). To this solution, 941 μl of Ambion ToTALLY RNA™ Lysis/Denaturation Solution (Ambion, Austin, Tex.) and 59 μl 3M sodium acetate, pH 5.5 (Ambion) was added, followed by mixing. After incubation at room temperature for 15 minutes, 770 μl of acid phenol/chloroform (Ambion) was added and the tubes were mixed by vortexing. The solution was transferred to 2 ml plastic screw capped tubes and incubated for 5 minutes at room temperature. The phenol extractions were spun for 1 minute at full speed in a microfuge (approximately 13,000×G) and the aqueous layer (1100 μl) was removed to a new tube containing 550 μl of 100% ethanol. After mixing, the solution was applied to one well of an Ambion RNAqueous®-96 Automated Kit filter plate and the RNA purified following the manufacturer's protocol. Following RNA elution, the sample was treated with DNase I (Invitrogen, Carlsbad, Calif.) a second time to remove residue genomic DNA. The RNA was incubated in 1×DNase digestion buffer, plus 3 units of enzyme for one hour at room temperature. The enzyme was inactived by the addition of EDTA to a final concentration of 13 mM followed by heating at 68° C. for 10 minutes. The mixture was desalted by passage over a MultiScreen® PCR_{micro96} plate (Millipore, Billerica, Mass.) and eluted in 50 μl of water. A 1 μl aliquot of the RNA was analyzed on the Agilent 2100 Bioanalyzer (Agilent, Waldbronn, Germany) and the remainder was stored at −80° C. The quality of the RNA sample was assessed using the RIN value calculated by the Bioanalyzer software.

cDNA Synthesis

The synthesis of cDNA was accomplished by mixing approximately 1 μg of total RNA with 1.5 μl random hexamers (Invitrogen, 500 ng/μl) in a final volume of 16.5 μl. Following incubation at 75° C. for 10 minutes and 25° C. for 10 minutes, 6 μl of first strand buffer (Invitrogen), 1.5 μl of 10 mM dNTPs (Invitrogen, 10 mM each dNTP), 1.25 μl Superscript II™ (Invitrogen, 200 units/ul), and 4 μl water were added. The final reaction volume was 30 μl and incubation was carried out at 25° C. for 10 minutes, 42° C. for 1 hour, and 95° C. for 10 minutes. Reactions were chilled to 4° C. until adding 70 μl of water followed by purification with a MultiScreen®PCR_{micro96} plate. Elution of cDNA was carried out with 100 μl of water and the resulting material was stored at −20° C. until quantitation. In some cases the volume of the cDNA reaction was doubled to increase the yield of material.

Quantification of cDNA

A dye intercalation assay was used to determine cDNA yields. 5 μl of cDNA is mixed with 7 μl of 0.5N NaOH, 50 mM EDTA in a final volume of 47 μl. The mixture was incubated at 65° C. for 1 hour to hydrolyze the RNA, and then neutralized by the addition of 10 μl of 1M Tris, pH7. The cDNA concentration in 25 μl aliquots of the hydrolysis reaction was measured using Quant-it™ Oligreen®ssDNA reagent (Invitrogen) according to the manufacturer's instructions. Unknown samples were compared to a standard curve generated using single stranded DNA of known concentration. All fluorescence readings were made using a Fusion™ alpha instrument (Packard, Meridan, Conn.). The values obtained from duplicate hydrolysis reactions were averaged for each unknown cDNA sample. If the duplicates were not within 15% of each other, a third sample was run, compared to the prior two determinations, and the two most similar values averaged.

Quantitative Polymerase Chain Reaction (qPCR)

All qPCR runs were performed on either an Applied Biosystems 7900HT Fast Real Time PCR System (Applied Biosystems, Foster City, Calif.) or an MX3000P® (Stratagene, La Jolla, Calif.), using the primer/probe sets shown in Tables 1A and 1B. All probes were labeled with FAM™ (Applera, Norwalk, Conn.) at the 5′ end and BHQ-1® quencher at the 3′ end and were synthesized by Biosearch (Novato, Calif.). Each primer/probe set was checked to insure that the efficiency of PCR amplification was approximately 100% over the expression range of the assay. Replica plates (96 well format) were constructed containing either 1 ng or 10 ng of cDNA per well from each human donor. The plates also contain 2 negative control wells (“NTC”, water only) and 3 wells of pooled, commercial cDNA derived from the blood of 10 individuals (reference cDNA). Each qPCR reaction was 25 μl (final volume) and contained the following components: 12.5 μl Brilliant QPCR Master Mix® (Stratagene), 400 nM forward primer, 400 nM reverse primer, 50 nM probe, and 60 nM/300 nM ROX™ (Applera) (MX3000P® 7900HT instrument). The cycling conditions were 95° C., 10 minutes followed by 40 cycles of 95° C., 15 seconds; 60° C., 1 minute. Duplicate qPCR runs were performed for each gene. Rarely, when the replicate plates for a gene were not sufficiently in agreement, a third qPCR plate was run. Depending on the Ct values obtained, either the values from all three plates were averaged or the odd plate was excluded from further analysis.

The instrument used for the qPCR run dictated the preliminary data analysis steps. However, in each case the aim was to set the amplification threshold near the midpoint of the amplification curve with the same threshold being used for all samples on a given plate. The threshold was similar, although not necessarily identical, for duplicate plates run for the same gene. For the MX3000P®, the following settings were used to initially determine the threshold: smoothing parameter=5, baseline calculation employing the MX4000 algorithm, and background-based threshold using cycles 6 through 14 with a sigma multiplier of 20. Minor adjustments of the threshold were made manually, if needed, to place it roughly in the middle of the amplification plot. For plates run on the 7900HT the instrument's default settings were used to initially set the threshold. Manual adjustments were made thereafter, if needed.

TABLE 1A
Primer/probe sequences for selected genes/biomarkers.

| | | Gene | |
|---|---|---|---|
| | | Acces- | |
| | | sion | |
| | | Number | |
| | Abbre- | (SEQ ID | Representative |
| Gene Name | viation | NO:) | Primer/probe sequences (5′ to 3′)^{†} |
| adenosine | ADA | NM_000022 | F = GGTGGTGGAGCTGTGTAAGAAGTAC (SEQ ID NO: 1) |
| deaminase | | (SEQ ID | R = CTTCCTGGGATGGTCTCATCTC (SEQ ID NO: 2) |
| | | NO: 88) | P = CAGCAGACCGTGGTAGCCATTGACCT (SEQ ID NO: 3) |
| beta- | ARRB1 | L04685 | F = AGACACGAACTTGGCCTCTAGC (SEQ ID NO: 4) |
| arrestin 1 | | (SEQ ID | R = TTGTAGGAAACAATGATCCCCAG (SEQ ID NO: 5) |
| | | NO: 89) | P = TTGAGGGAAGGTGCCAACCGTGAGAT (SEQ ID NO: 6) |
| beta- | ARRB2 | BC007427 | F = TCTTCCATGCTCCGTCACAC (SEQ ID NO: 7) |
| arrestin 2 | | (SEQ ID | R = CGAATCTCAAAGTCTACGCCG (SEQ ID NO: 8) |
| | | NO: 90) | P = AGCCAGGCCCAGAGGATACAGGAAA (SEQ ID NO: 9) |
| CD8 alpha | CD8a | M12824 | F = TTCCGCCGAGAGAACGAG (SEQ ID NO: 10) |
| | | (SEQ ID | R = AAGACCGGCACGAAGTGG (SEQ ID NO: 11) |
| | | NO: 91) | P = TCGGCCCTGAGCAACTCCATCATGTA (SEQ ID NO: 12) |
| CD8 beta | CD8b | M37601 | F = TGACAGTCACCACGAGTTCCTG (SEQ ID NO: 13) |
| | | (SEQ ID | R = TCTCCTGTTCCACCTCTTCACC (SEQ ID NO: 14) |
| | | NO: 92) | P = CTCTGGGATTCCGCAAAAGGGACTAT (SEQ ID NO: 15) |
| cAMP responsive | CREB1 | NM_134442 | F = CTGGCTAACAATGGTACCGATG (SEQ ID NO: 16) |
| element binding | | (SEQ ID | R = GTGGTCTGTGCATACTGTAGAATGG (SEQ ID NO: 17) |
| protein 1 | | NO: 93) | P = CATGACCAATGCAGCAGCCACTCA (SEQ ID NO: 18) |
| cAMP responsive | CREB2 | M86842 | F = CACGTTGGATGACACTTGTGATC (SEQ ID NO: 19) |
| element binding | | (SEQ ID | R = CTGGGAGATGGCCAATTGG (SEQ ID NO: 20) |
| protein 2 | | NO: 94) | P = ACTAATAAGCAGCCCCCCCAGACGGT (SEQ ID NO: 21) |
| dipeptidyl | DPP4 | M74777 | F = GTGTCATTCAGTAAAGAGGCGAAG (SEQ ID NO: 22) |
| peptidase IV | | (SEQ ID | R = CTCAGCCCTTTATCATTCACGC (SEQ ID NO: 23) |
| | | NO: 95) | P = TTCCGGTCCTGGTCTGCCCCTCTATA (SEQ ID NO: 24) |
| extracellular | ERK1 | M84490 | F = TGACGGAGTATGTGGCTACGC (SEQ ID NO: 25) |
| signal-regulated | | (SEQ ID | R = CCACAGACCAGATGTCGATGG (SEQ ID NO: 26) |
| kinase 1 | | NO: 96) | P = CTGGTACCGGGCCCCAGAGATCAT (SEQ ID NO: 27) |
| extracellular | ERK2 | M84489 | F = TAACGTTCTGCACCGTGACC (SEQ ID NO: 28) |
| signal-regulated | | (SEQ ID | R = CAGGCCAAAGTCACAGATCTTG (SEQ ID NO: 29) |
| kinase 2 | | NO: 97) | P = ACCTGCTGCTCAACACCACCTGTGAT (SEQ ID NO: 30) |
| guanine nucleotide | Gi2 | X04828 | F = AGGCGTGCTCCCTGATGAC (SEQ ID NO: 31) |
| binding protein | | (SEQ ID | R = GCTCCAGGTCGTTCAGGTAGTAG (SEQ ID NO: 32) |
| alpha i2 | | NO: 98) | P = AGGCCTGCTTTGGCCGCTCAA (SEQ ID NO: 33) |
| guanine nucleotide | Gs | AF493897 | F = GACTATGTGCCGAGCGATCAG (SEQ ID NO: 34) |
| binding protein | | (SEQ ID | R = GTCCACCTGGAACTTGGTCTCA (SEQ ID NO: 35) |
| alpha s(long) | | NO: 99) | P = CTGCTTCGCTGCCGTGTCCTGA (SEQ ID NO: 36) |
| alpha- | GR | X03225 | F = TCCCTGGTCGAACAGTTTTTTC (SEQ ID NO: 37) |
| glucocorticoid | | (SEQ ID | R = TTTGGGAGGTGGTCCTGTTG (SEQ ID NO: 38) |
| receptor | | NO: 100) | P = TGTAAGCTCTCCTCCATCCAGCTCCTCAA |
| | | | (SEQ ID NO: 39) |
| interleukin 1, | IL1b | NM_000576 | F = GATGGCCCTAAACAGATGAAGTG (SEQ ID NO: 40) |
| beta | | (SEQ ID | R = CCTGAAGCCCTTGCTGTAGTG (SEQ ID NO: 41) |
| | | NO: 101) | P = ATGGCGGCATCCAGCTACGAATCTC (SEQ ID NO: 42) |
| interleukin 6 | IL6 | M14584 | F = AGCCACTCACCTCTTCAGAACG (SEQ ID NO: 43) |
| | | (SEQ ID | R = CATGTCTCCTTTCTCAGGGCTG (SEQ ID NO: 44) |
| | | NO: 102) | P = CAAATTCGGTACATCCTCGACGGCAT (SEQ ID NO: 45) |
| interleukin 8 | IL8 | M28130 | F = CTGCTAGCCAGGATCCACAAG (SEQ ID NO: 46) |
| | | (SEQ ID | R = CTGTGAGGTAAGATGGTGGCTAATAC (SEQ ID NO: 47) |
| | | NO: 103) | P = CTTGTTCCACTGTGCCTTGGTTTCTCCTT |
| | | | (SEQ ID NO: 48) |
| indoleamine- | INDO | NM_002164 | F = GCTTCGAGAAAGAGTTGAGAAGTTAAAC |
| pyrrole 2,3 | | | (SEQ ID NO: 49) |
| dioxygenase | | (SEQ ID | R = GACCTTTGCCCCACACATATG (SEQ ID NO: 50) |
| | | NO: 104) | P = CTCACAGACCACAAGTCACAGCGCCTT |
| | | | (SEQ ID NO: 51) |
| p38 mitogen | MAPK14 | L35253 | F = CGGCAGGAGCTGAACAAGAC (SEQ ID NO: 52) |
| activated protein | | (SEQ ID | R = AGCAGCACACACAGAGCCATAG (SEQ ID NO: 53) |
| kinase 14 | | NO: 105) | P = CCGAGCGTTACCAGAACCTGTCTCCA (SEQ ID NO: 54) |
| mitogen-activated | MAPK8 | AY893269 | F = CCAACACCCGTACATCAATGTC (SEQ ID NO: 55) |
| protein | | (SEQ ID | R = CACTCTTCTATTGTGTGTTCCCTTTC (SEQ ID NO: 56) |
| kinase 8 | | NO: 106) | P = CACCACCAAAGATCCCTGACAAGCAGTT |
| | | | (SEQ ID NO: 57) |
| map kinase | MKP1 | X68277 | F = GCCAGGCAGGCATTTCC (SEQ ID NO: 58) |
| phosphatase 1 | | (SEQ ID | R = ATGCTTCGCCTCTGCTTCAC (SEQ ID NO: 59) |
| | | NO: 107) | P = TCAGCCACCATCTGCCTTGCTTACCTT |
| | | | (SEQ ID NO: 60) |
| mineralocorticoid | MR | M16801 | F = AGCCCAGAGGAAGGGACAAC (SEQ ID NO: 61) |
| receptor | | (SEQ ID | R = TGTGAGCGCTCGTGAGATTG (SEQ ID NO: 62) |
| | | NO: 108) | P = CTCCTGCAAAAGAACCCTCGGTCAACA |
| | | | (SEQ ID NO: 63) |
| ornithine | ODC1 | NM_002539 | F = CCATGTAGGAAGCGGCTGTAC (SEQ ID NO: 64) |
| decarboxylase 1 | | (SEQ ID | R = TCAGCCCCCATGTCAAAAAC (SEQ ID NO: 65) |
| | | NO: 109) | P = ATCCTGAGACCTTCGTGCAGGCAATCT |
| | | | (SEQ ID NO: 66) |
| purinergic | P2X7 | NM_002562 | F = GCTGTCGCTCCCATATTTATCC (SEQ ID NO: 67) |
| receptor P2X7 | | (SEQ ID | R = CACAATGGACTCGCACTTCTTC (SEQ ID NO: 68) |
| | | NO: 110) | P = CTGTCAGCCCTGTGTGGTCAACGAATAC |
| | | | (SEQ ID NO: 69) |
| benzodiazapine | PBR | BC001110 | F = CTGGTCTGGAAAGAGCTGGG (SEQ ID NO: 70) |
| receptor | | (SEQ ID | R = CAGCAGGAGATCCACCAAGG (SEQ ID NO: 71) |
| (peripheral-type) | | NO: 111) | P = CCCCATCTTCTTTGGTGCCCGAC (SEQ ID NO: 72) |
| prolyl | PREP | D21102 | F = GGGAATATGACTACGTGACCAATG (SEQ ID NO: 73) |
| endopeptidase | | (SEQ ID | R = GGATCCCTGAAGTCAATGTTGATC (SEQ ID NO: 74) |
| | | NO: 112) | P = CATTCAAGACGAATCGCCAGTCTCCC (SEQ ID NO: 75) |
| regulator of | RGS2 | NM_002923 | F = GATTGGAAGACCCGTTTGAGC (SEQ ID NO: 76) |
| G-protein | | (SEQ ID | R = CAGGAGAAGGCTTGATGAAAGC (SEQ ID NO: 77) |
| signaling 2 | | NO: 113) | P = CTGGGAAGCCCAAAACCGGCAA (SEQ ID NO: 78) |
| S100 calcium | S100A10 | NM_002966 | F = AGGAGTTCCCTGGATTTTTGG (SEQ ID NO: 79) |
| binding protein | | (SEQ ID | R = GCCCACTTTGCCATCTCTACAC (SEQ ID NO: 80) |
| A10 (p11) | | NO: 114) | P = CAAAAAGACCCTCTGGCTGTGGACAAAA |
| | | | (SEQ ID NO: 81) |
| serotonin | SERT | NM_001045 | F = CATGGCTGAGATGAGGAATGAAG (SEQ ID NO: 82) |
| transporter | | (SEQ ID | R = GCTGGCATGTTGGCTATCG (SEQ ID NO: 83) |
| | | NO: 115) | P = ACGCAGGTCCCAGCCTCCTCTTCAT (SEQ ID NO: 84) |
| vesicle | VMAT2 | L23205 | F = TGGATTCGTCAATGATGCCTATC (SEQ ID NO: 85) |
| monoamine | | (SEQ ID | R = ATGCCACATCCGCAATGG (SEQ ID NO: 86) |
| transporter 2 | | NO: 116) | P = AGACCTGCGGCACGTGTCCGTCTA (SEQ ID NO: 87) |

^{†}F = Forward primer sequence;
R = Reverse primer sequence;
P = Probe sequence

### Normalization of Gene Expression

In order to effectively compare gene expression profiles between different samples, it is preferable to control for variables that could mask any underlying biological changes. For example, day to day differences in the efficiency of enzymatic reactions, instrumentation performance, and pipeting will all influence the signal obtained on a given day. The preferred way to minimize the influence of these variables is through the use of multiple normalization genes (Andersen, C. L. et al., Cancer Res, 2004, 64:5245-5250; Jin, P. et al., BMC Genomics, 2004, 5:55; Huggett, J. et al., Genes and Immunity, 2005, 6:279-284). The ideal normalization gene is expressed at a conveniently measured level and is unchanged by manipulations that are part of the experimental design. Although the use of normalization genes is commonplace, researchers have often not verified whether the genes they use are stably expressed in their experimental system. To avoid this problem, a commercially available software program GeNorm™ (PrimerDesign Ltd., Southhampton, UK) was used. The method is based on the work published by Vandesompele, J. et al., Genome Biol, 2002, 3(7): RESEARCH0034.1-0034.11 (Epub Jun. 18, 2002) and allows one to determine if a candidate normalization gene is stably expressed or not. To select normalization genes, the literature was first scanned to identify genes that previously had been used by investigators to normalize gene expression in humans, with an emphasis on experiments conducted with blood samples (Vandesompele, J. et al. Genome Biol, Epub Jun. 18, 2002, 3(7): RESEARCH0034.1-0034.11, especially at page 0034.5, table 3; Applied Biosystems Application Note 2006, publication 127AP08-01, especially at page 3, FIG. 1). From this search, the genes shown in Table 1B were identified. To confirm that these genes were valid for normalization in the present experiments, the expression profile of seven genes was analyzed with Genorm™ using blood samples derived from different experimental sets, including normal subjects, depressed patients without drug treatment and depressed patients with drug treatment. In all sets, the combination of seven genes achieved good normalization, as determined by a pair wise variation value (V) of 0.15 or less (Vandesompele, J. et al., Genome Biol, Epub Jun. 18, 2002, 3(7): RESEARCH0034.1-0034.11).

Although Genorm™ states that it is only necessary to use the two or three best genes for normalization, a combination of more than three normalization genes should be considered for several reasons. First, using more normalization genes will aid in prediction considering that new drug treatments, genetic backgrounds, or disease states may influence the expression of normalization genes. More than three normalization genes are expected to improve the process by dampening the influence of any gene that is not stably expressed in a particular experiment. Also, by consistently using more than three genes to normalize expression data, expression results can be compared from all studies conducted over time. Because clinical samples do not always come matched with appropriate controls, the use of more than three normalization genes is an important consideration. While normalization with more than three genes is the preferred method when comparing gene expression across different experiments, it is still valid to use two or three genes within any particular experiment, provided all samples being compared are treated in the same manner.

TABLE 1B
Normalization genes.

| | | Gene Accession |
|---|---|---|
| | | Number |
| Gene Name | Abbreviation | (SEQ ID NO:) |
| beta-actin | ACTB | NM_001101 |
| | | (SEQ ID NO: 117) |
| beta-2-microglobulin | B2M | NM_004048 |
| | | (SEQ ID NO: 118) |
| glyceraldehyde-3-phosphate | GAPD | NM_002046 |
| dehydrogenase | | (SEQ ID NO: 119) |
| glucuronidase, beta | GUSB | NM_000181 |
| | | (SEQ ID NO: 120) |
| hydroxymethyl-bilane synthase | HMBS | NM_000190 |
| | | (SEQ ID NO: 121) |
| hypoxanthine phosphoribosyl- | HPRT1 | NM_000194 |
| transferase I | | (SEQ ID NO: 122) |
| phosphoglycerate kinase | PGK1 | NM_000291 |
| | | (SEQ ID NO: 123) |
| peptidylpropyl isomerase A | PPIA | NM_021130 |
| (cyclophilin A) | | (SEQ ID NO: 124) |
| ribosomal protein, large, P0 | RPLP0 | NM_001002 |
| | | (SEQ ID NO: 125) |
| ribosomal protein L13a | RPL13A | NM_012423 |
| | | (SEQ ID NO: 126) |
| succinate dehydrogenase | SDHA | NM_004168 |
| complex, subunit A | | (SEQ ID NO: 127) |
| TATA box binding protein | TBP | NM_003194 |
| (transcription factor IID) | | (M34960) |
| | | (SEQ ID NO: 128) |
| transferring receptor | TFRC | NM_003234 |
| (p90, CD71) | | (SEQ ID NO: 129) |
| ubiquitin C | UBC | NM_021009 |
| | | (M26880) |
| | | (SEQ ID NO: 130) |
| tyrosine 3-monooxygenase/ | YWHAZ | NM_003406 |
| tryptophan | | (SEQ ID NO: 131) |
| 5-monooxygenase activation | | |
| protein, zeta polypeptide | | |
| eukaryotic 18S ribosomal RNA | 18S | X03205 |
| | | (SEQ ID NO: 132) |

As described in section 5.4.1.2 above, primers may be designed for any of the genes described herein. The publicly available sequences for the genes identified in Table 1A and Table 1B are indicated by Gene Accession Number (GenBank database) and incorporated herein by reference in their entirety. The sequences for the genes identified in Table 1A and Table 1B are disclosed in the accompanying Sequence Listing as listed by the appropriate SEQ ID NO given in the Table.

### Transcriptional Data Analysis

The average Ct (cycle threshold) values for each unknown sample, derived from duplicate PCR plates, were determined for each gene. In a real time PCR assay, a positive reaction is detected by accumulation of a fluorescent signal. The Ct is defined as the number of cycles required for the fluorescent signal to cross the threshold (i.e. exceeds background level). Ct levels are inversely proportional to the amount of target nucleic acid in the sample (i.e. the lower the Ct level the greater the amount of target nucleic acid in the sample).

The relative expression level for each unknown cDNA sample, as well as the reference cDNA, was calculated by the 2^{-delta Ct} method (Livak, K. and Schmittgen, T., Methods, 2001, 25:402-408) using the average Cts from the seven normalization genes. Next, setting the relative expression level of the reference cDNA at 100%, all other samples were then expressed as a percentage of the reference. Finally, these percentages were converted to copies per ng of cDNA by multiplying the percentage by the number of copies of each gene contained in the reference cDNA.

### Univariate Statistical Analysis and Graphing

Correlations between gene expression values and clinical parameters derived from patient/subject questionnaires were investigated using the R statistical package. The questionnaire data was coded, as necessary, to facilitate comparisons. The gene expression data was log transformed prior to analysis and both parametric and non-parametric analyses were performed. The threshold for significance was set at p<0.05. See, for example, Table 3. Univariate tests were used to determine whether particular genes are consistently up- or down-regulated for a given population of subjects.

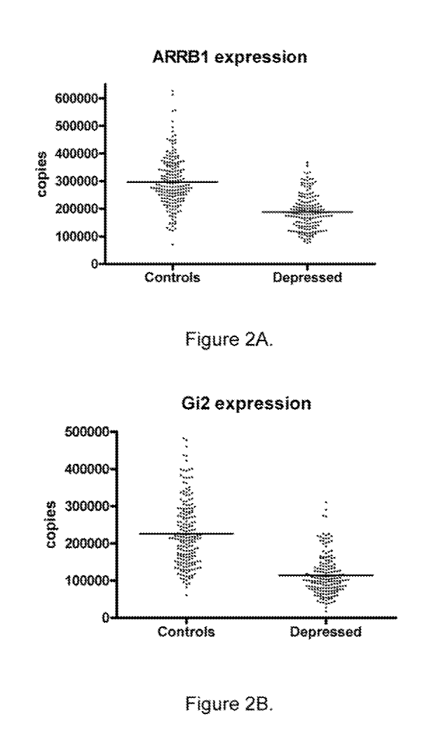

Scatter plots and the associated univariate statistical analyses comparing expression levels between control subjects and depressed patients were generated for each gene using GraphPad Prism4® (GraphPad Software, Inc, San Diego, Calif.). Because the gene expression values are not necessarily normally distributed, the non-parametric Mann-Whitney test was used to compare the groups. The significance threshold was set at p<0.05. Certain genes, and their relative expression levels in blood, are exemplified in FIGS. 2 through 7.

### Multivariate Analyses

In order to differentiate diseased patients from healthy control subjects, classification algorithms were used. A classification algorithm, typically a machine learning algorithm, runs through the following two steps: (1) selects a subset of genes from an mRNA transcription data set, whose gene expression levels collectively are found to be the most informative; (2) trains and returns a pre-selected type of classification algorithm trained on a subset of genes as identified in step (1).

(1) Selection of Genes

In the first step, mRNA transcription data sets from healthy control subjects and depressed subjects, or other diseased subjects, were used collectively as input to a Random Forest algorithm (Breiman, L., 2001, Machine Learning 45(1):5-32)). Each data set representing mRNA transcription data from each subject's blood sample based on the genes listed in Table 1A and methods described herein. By successively eliminating the least important genes, the Random Forest algorithm returns a list containing the most important genes using the out-of-bag (OOB) error minimization criterion (Liaw, A, and Wiener, M. December 2002, Classification and regression by random Forest. R News Vol. 2/3: 18-22).

### (2) Training and Classification

In the second step, a Support Vector Machine classification algorithm (Cortes, C. and Vapnik, V. 1995, Machine Learning, 20(3):273-97), or the like, was tuned using the transcription profiles associated with the most important genes identified as in step (1) and trained based on cross-validation.

In another method, Stepwise Logistic Regression was used for both step (1), selecting the most important or explanatory genes, and step (2), training the algorithm for classification via cross-validation.

In other analyses, the RVM classifier was used, along with a Genetic algorithm. Data sets were trained with the RVM algorithm, and the Genetic algorithm evaluated a large number of RVMs which were trained and tested on different subsets of candidate variables to identify the possible gene-interactions. The performance of each variable subset was evaluated through cross validation.

During the training step, a cross validation method, such as a leave-one-out cross validation (LOOCV) or ten-fold cross validation, was performed by the algorithm. Cross validation is the statistical practice of separating samples of data into distinct subsets such that the analysis is initially performed on a single subset, while the other subset(s) are retained for subsequent use in confirming and validating the initial analysis. The initial subset of data is a training set; the other subset(s) are validation or testing sets which are treated as unknowns in order to determine their classification.

For example, the data from all samples (N) is split into two distinct subsets wherein one subset of data (m) is used for validation of the samples, i.e. subset m is used as a set of unknowns. The remaining subset (N-m) trains the classification algorithm. Such cross-validation (CV) method is repeated until all data sets are treated as unknowns. Values of accuracy and predictive value may be calculated based on whether each of the samples treated as unknowns classify correctly or not.

In one such cross validation method, the classification algorithm was trained with 90% of the sample data sets, and the classification of the remaining 10% of the sample data is predicted by the trained algorithm. Such 10-fold CV is repeated 10 times. Cross validation can illustrate the “operating curve”, i.e. that the trained classification algorithm performs better than some random selection process, for example better than chance. To estimate the classification error of a classification algorithm built according to the prescriptions given in (1) and (2) above, calculations were made for accuracy, positive predictive value (PPV), and negative predictive value (PPV) to determine how well the trained classification algorithm has performed.

The accuracy of a trained classification algorithm is the total number of correct classifications out of the total number of samples.

By the above method, the number of data sets (i.e. subjects) that scored correctly in the “diseased” class gives a measure of the positive predictive value (PPV). The PPV, also called precision rate, or post-test probability of disease, is the proportion of patients with positive test results who were correctly diagnosed.

Also by the above method, the number of data sets (i.e. subjects) that scored correctly in the “healthy” or “control” group gives a measure of the negative predictive value (NPV). The negative predictive value is the proportion of patients with negative test results who were correctly diagnosed.

Analysis of randomized (permuted) data sets.

To determine if the classification accuracies obtained using SLR or SVM were meaningful, i.e. better than chance, each data set was further analyzed as follows:

a) The accuracies for the original data sets were obtained by the methods explained hereinabove.
b) Three new permuted data sets were created, wherein the assignment for each individual sample is randomly assigned, while still maintaining the same percentage of patients as in the original data set.
c) Accuracies were then calculated for each randomized data set.
d) The 10 accuracies (from 10-fold CV of the original data set) was compared with the 30 permuted accuracies (3 random sets having undergone under 10-fold CV) using a Mann Whitney test.
e) Comparisons producing p values less than 0.01 were interpreted to mean the accuracies from the original data set are not due to random chance, i.e. the control and patient groups can be separated. Comparisons producing p values greater than 0.01 are deemed random, meaning the patient and control groups are not convincingly separable.

### Patients/Subjects Used for Transcription Profile Identification

One goal of these studies was to define, correlate and link transcription profiles identified in blood of normal donors with subgroups that may help identify phenotypes that are at risk for neuropsychiatric disorders, such as affective disorders. Once a baseline transcription profile of normal donors had been established, comparisons were made between the normal population and patients with clinically diagnosed depression, severe depression, bipolar disorder, BPD or PTSD. Another goal of these studies was to identify profiles that could classify subjects as either normal controls or patients with an affective disorder such as depression, severe depression, bipolar disorder, BPD or PTSD.

In order to determine the presence of subgroups within the normal population, e.g. subjects with a risk profile, and to be able to correlate subgroups with transcription profiles in whole blood, a baseline database of normal volunteers was established.

### Control Patients/Subjects (United States)

500 blood samples were collected from normal volunteers donating blood at blood banks serving the southeastern Pennsylvania and Delaware regions. Informed consent was obtained from all donors. Personal information was irreversibly anonymized.

Donors were restricted to Caucasians to minimize variance within the population. Within the population donors were split evenly between genders. There were no additional exclusion factors above those used by the blood bank for donors. All donors were required to fill out a questionnaire to help characterize general physical condition, medical problems, drug use and abuse, family history, and psychiatric problems. Elements of the questionnaire were based on standard psychiatric measures that are available in the public domain. Answers on the questionnaire were self reported and the donors did not receive a medical or psychiatric evaluation. The questionnaire covered multiple factors including those factors categorized in Table 2.

TABLE 2

| | | | Psychiatric | |
|---|---|---|---|---|
| | General | Family | history/life | Depressive |
| Demographic | medical | history | experience | symptoms |
| race | height/ | suicide of | presence/ | change in |
| | weight | relative | severity | vegetative |
| | | | of stressful | functions |
| | | | life events | |
| | | | in past/ | |
| | | | recent | |
| gender | current/past | family | previous | changes in |
| | medications | psychiatric | diagnosis of | cognitive |
| | | history | psychiatric | functions |
| | | | illness | |
| marital | current/past | | | anxiety/ |
| status | medical | | | panic |
| | problems | | | attacks |
| employment | surgeries | | | |
| status | | | | |
| occupation | tobacco use | | | |
| meal frequency | alcohol use | | | |
| | drugs of | | | |
| | abuse | | | |

The extensive questionnaire was used to obtain data on multiple factors in a donor's history or present medical condition that may increase their risk of future psychiatric disorders and to associate a unique transcription profile to a specific phenotype identified using the questionnaire. This data was used to segment the normal population and identify segments within the depressed patients more reliably and consistently than by using currently available methodologies. Factors that were evaluated include (but are not limited to): severity of recent stressful life events, presence and severity of early life stress, family history of psychiatric disorders and a group of pro-depressive vegetative symptoms including changes in appetite and sleep patterns. Where necessary, scores from multiple groups of questions were combined to assess impact of multiple negative factors, i.e. symptom scores.

To avoid the confound of common factors, such as smoking, or body mass index (BMI), which may be considered extremes that can potentially affect blood transcription profiles, questionnaire data was used to group donors by identifiable patterns in demographic, personal or medical attributes. These factors were evaluated independently to assess their effect on transcription profiles. Identification and segmentation of donors was according to non-psychiatric factors to evaluate their effects on transcription profiles as these could be confounds in the identification of pro-depressive phenotypes, wherein such factors include: BMI, smoking, alcohol abuse, drug use (and abuse). Effects of other factors were also evaluated.

### Control Patients/Subjects (Denmark)

200 subjects were selected from an initial collection of blood from approximately 1000 healthy volunteers (control subjects), based on Danish ethnic origin (going back two generations) and geographically covering Denmark. Thus, data regarding birthplace (and that of parents and grandparents) was obtained. General health status and psychiatric history were initially obtained. Psychiatric history information was supplemented with a short screen for previous episodes of depression. The cohort of 200 control subjects resulted in an equivalent distribution of men and women with an average age of approximately 40 years (range 18-65 years.). Each subject was exposed to a minor physical examination, including assessment of height, weight, measure of the circumference of the abdomen and the hips and EKG. Each subject completed detailed questionnaire in which they characterized regarding certain traits of personality and a more thorough family history of medical and psychiatric illnesses. (See Table 2.)

Using the data provided by the control subjects as mentioned above, the normal population was segmented and specific phenotypes were associated with changes in transcription profiles identified in peripheral blood. See Tables 3A and 3B.

### Control Patients/Subjects (United Kingdom)

Blood samples were collected from healthy volunteers participating in a controlled clinical study in the United Kingdom. Informed consent was obtained from all donors. Men and women were included in the study. Women were included if using an accepted method of contraception (double-barrier contraception), had been surgically sterilised, or are post-menopausal (defined as 2 years without menses)—oral contraceptives were not allowed. The subjects included are ≧18 years of age and ≦45 years of age, but less than ≧65 years of age. Each subject included in the study is in good health, in the opinion of the investigator, on the basis of a pre-study physical examination, medical history, vital signs, ECG, and the results of blood biochemistry, haematology, and serology tests, and a urinalysis.

### Identification of Transcription Profiles in Depressed Patients

To assess the changes in transcription profiles in depressed patients, blood from depressed patients, i.e. patients suffering from a major depressive disorder (MDD), was obtained in a controlled clinical study. Informed consent was obtained from all donors.

### Patient Selection Criteria:

Patients/subjects eligible for the study were outpatients, males or females, suffering from moderate MDD having a MADRS total score 26 and a CGI-S score 4 at the baseline visit. The primary diagnosis of MDD must be according to DSM-IV-TR® criteria. Patients are aged 18 to 65 years (extremes included) and recruited from psychiatric outpatient clinics and general practitioners. Patients who suffer from a secondary co-morbid anxiety disorder, except Obsessive-Compulsive Disorder (OCD), Post-traumatic Stress Disorder (PTSD), or Panic Disorder (PD) (DSM-IV-TR® criteria) could be included in the study. Furthermore, the patient, in the opinion of the investigator, was otherwise healthy on the basis of a physical examination, medical history and vital signs. Patients, in the opinion of the investigator, that were unlikely to comply with the clinical study protocol or were unsuitable for any reason, may be excluded from the study.

### Identification of Transcription Profiles in Depressed Patients

To assess the changes in transcription profiles in patients suffering from a severe major depressive disorder (SMDD), blood from these patients was obtained in a controlled clinical study. Informed consent was obtained from all donors.

### Patient Selection Criteria:

Patients/subjects eligible for this study were outpatients suffering from SMDD, recruited from psychiatric outpatient clinics, males or females, aged between 18 and 65 years (extremes included). All patients included in this study should have had a MADRS total score of 30 or above (i.e. more severely depressed patients). The chosen patient suffers from a major depressive episode (MDE) as primary diagnosis according to DSM IV-TR® criteria (current episode assessed with the Mini International Neuropsychiatric Interview (MINI)). The reported duration of the current MDE is at least 3 months and less than 12 months at baseline. Patients are included/excluded from the study based on the criteria as explained above with respect to moderately depressed patients. Patients, in the opinion of the investigator, unlikely to comply with the clinical study protocol or unsuitable for any reason, could be excluded from the study.

### Identification of Transcription Profiles in Bipolar Patients

To assess the changes in transcription profiles in bipolar patients, blood from bipolar patients was obtained. These patients had undergone extensive evaluation by a psychiatrist and were under medical care. Informed consent was obtained from all donors.

### Patient Selection Criteria:

Before a patient/subject could donate blood under this protocol the following criteria must have been fulfilled:

a) Patient has been diagnosed with moderate or severe major depression or bipolar I according to DSM IV-TR®. Eighty-seven percent of the patients met the DSM IV-TR® criteria for bipolar I disorder.
b) At the time of blood collection, patient is not taking any psychopharmacological drugs and has not taken any psychopharmacological drugs for at least 2 weeks. In addition, none of the patients has been treated with fluoxetine, irreversible MACH or depot neuroleptics for at least 2 months.
c) Patient is not suffering from other acute psychiatric symptoms, e.g. substance abuse.
d) Whenever possible, blood samples from female patients should be collected within 2 weeks of start of menstruation. In any cast, the date of the first day of the last menstrual period will be recorded.
e) Patient has not taken any illicit drugs/drugs of abuse during the last 6 months.
f) Patient has not abused alcohol during the last 6 months.
g) Female patient is not pregnant and not breastfeeding.
h) Patient is currently (including the last week) not suffering from any other acute general medical condition (including minor conditions, e.g. common cold).
i) Patient does currently (including the last week) not take any regular medication (including oral contraceptives, herbal therapies, nutritional supplements, vitamins).
j) Patient should not have taken any medication (including oral contraceptives, herbal therapies, nutritional supplements, vitamins) within the week prior to the blood sample collection. If a drug was taken, e.g. for an acute headache, the blood sample collection should be delayed by one week.
k) If patient indicates tobacco use, information on average amount per day needs to be provided.
l) If patient indicates alcohol consumption without abuse, information on average amount per week needs to be provided.
m) Patient has returned the questionnaire accompanying the blood sample collection.
n) Patient has read and understood the patient information.
o) Patient has signed the informed consent.

From all patients donating blood under this protocol the following information must be obtained: a detailed psychiatric and general medical history, a psychiatric family history, a detailed clinical description of current symptoms, medication history for at least the last 3 months, and information on illicit and non-illicit drugs of abuse in at least the last 6 months.

### Identification of Transcription Profiles in Borderline Personality Disorder Patients

To assess the changes in transcription profiles in patients with borderline personality disorder (BPD), blood from borderline personality disorder patients was obtained. These patients had undergone extensive evaluation by a psychiatrist and were under medical care. Informed consent was obtained from all donors.

### Patient/Subject Selection Criteria for BPD Study:

Before a patient could donate blood under this protocol the following criteria must have been fulfilled:

a) Patient has been diagnosed with borderline personality disorder according to DSM-IV®.
b) For the untreated patients group, patient is not taking any psychopharmacological drugs and has not taken any psychopharmacological drugs for at least 2 weeks at the time of blood collection. Patients, who have in the past been treated with fluoxetine, irreversible MAOI or depot neuroleptics, have not taken any of these medications for at least 4 weeks prior to blood collection.
c) From a small cohort of patients (approximately 25 patients) blood samples will be collected during an acute psychiatric exacerbation of the primary psychiatric disorder (Borderline personality disorder). All other patients will not suffer from an acute psychiatric exacerbation at the time of blood collection. Only in patients in whom blood is sampled during an acute exacerbation, a second sample will be collected during remission. Whenever medically possible, the treatment at the two time points will be the same.
d) Patient is not suffering from other acute psychiatric symptoms, e.g. substance abuse.
e) Whenever possible, blood samples from female patients should be collected within 2 weeks of start of menstruation. In any case, the date of the first day of the last menstrual period will be recorded.
f) Patient has not taken any illicit drugs/drugs of abuse during the last 6 months.
g) Patient has not abused alcohol during the last 6 months.
h) Female patient is not pregnant and not breastfeeding.
i) Patient is currently (including the last week) not suffering from any other acute general medical condition (including minor conditions, e.g. common cold).
j) Patient does currently (including the last week) not take any regular medication (including oral contraceptives, herbal therapies, nutritional supplements, vitamins) other than prescribed venlafaxine or duloxetine.
k) If patient is treated with venlafaxine or duloxetine, treatment must have been given at the current dose for at least 3 months.
l) Patient should not have taken any medication (including oral contraceptives, herbal therapies, nutritional supplements, vitamins) within the week prior to the blood sample collection. If a drug was taken, e.g. for an acute headache, the blood sample collection should be delayed by one week.
m) If patient indicates tobacco use, information on average amount per day needs to be provided.
n) If patient indicates alcohol consumption without abuse, information on average amount per week needs to be provided.
o) Patient has returned the questionnaire accompanying the blood sample collection.
p) Patient has read and understood the patient information.
q) Patient has signed the informed consent.

From all patients donating blood under this protocol, a detailed psychiatric history, including a family history, clinical description and medication and drug record was obtained.

Patients completed a questionnaire developed to specifically address factors which can confound transcription profiles, e.g. drug use, general medical conditions. Patients returned the questionnaire to the investigator. The questionnaire was coded with the same code as the blood sample and other clinical data, to ensure that the patient's identity is not disclosed to personnel at the site of transcription analysis. The questionnaire was transferred to the site of the transcription analysis together with the blood samples.

### Transcription Profiles in Post Traumatic Stress Disorder (PTSD) Patients

To assess the changes in transcription profiles in patients with PTSD, blood from PTSD patients was obtained. These patients had undergone extensive evaluation by a psychiatrist and were under medical care. Informed consent was obtained from all donors.

### Patient/Subject Selection Criteria for PTSD Study:

Subjects for this study were males that met the following criteria:

a) Subject has been diagnosed with acute PTSD, or remitted PTSD (according to DSM-IV®), or has been exposed to trauma and not developed PTSD or is categorized as a control. Controls were selected for this study that were not exposed to trauma, and were originally from the same geographic area.
b) Patient is not taking any psychopharmacological drugs and has not taken any psychopharmacological drugs for at least 2 weeks at the time of blood collection. Patients, who have in the past been treated with fluoxetine, irreversible MAOI or depot neuroleptics, have not taken any of these medications for at least 4 weeks prior to blood collection.
c) Patient is not suffering from other acute psychiatric symptoms, e.g. substance abuse.
d) Patient has not taken any illicit drugs/drugs of abuse during the last 6 months.
e) Patient has not abused alcohol during the last 6 months.
f) Patient is currently (including the last week) not suffering from any other acute general medical condition (including minor conditions, e.g. common cold).
g) Patient should not have taken any medication (including herbal therapies, nutritional supplements, vitamins) within the week prior to the blood sample collection. If a drug was taken, e.g. for an acute headache, the blood sample collection should be delayed by one week.
h) If patient indicates tobacco use, information on average amount per day needs to be provided.
i) If patient indicates alcohol consumption without abuse, information on average amount per week needs to be provided.
j) Patient does currently (including the last week) not take any regular medication including herbal therapies, nutritional supplements, vitamins).

All clinical and demographic data as described above were collected at the site of blood collection before transferring the information to the site of the transcription analysis (Lundbeck Research USA, Inc., Paramus, N.J.). The exploratory analysis of any relationship between clinical characteristics and transcription profiles was performed without knowledge of the patient identity at Lundbeck Research USA.

### Results and Discussion

### Identification of Transcription Profiles in Control Subjects.

Gene expression levels for the 29 genes listed in Table 1A were measured in blood samples from control subjects, including subjects from two control groups (U.S. and DK).

Although these individuals are all healthy, trends of gene expression were identified that correlate with particular responses to questionnaire items. Such trends, if identified, might be exaggerated in the population of depressed patients.

Converting Questionnaire Responses into Coded Values for Statistical Analysis.

The self-assessed questionnaires filled out by the US and Danish control subjects contain similar, but not identical items. In order to use information from the questionnaires to search for possible associations between responses and gene expression data, it was necessary to code the information prior to statistical analysis.

Examples of the coding strategy are as follows:
a) Continuous variables such as age and BMI were used as reported by the subjects. Alternatively, the raw scores were combined into two or three bins (high, medium, low values) prior to analysis.
b) Gender was converted to a binary response (0, 1).
c) Questions regarding the frequency of symptoms linked to depression, such as difficulty sleeping, lack of energy, or feeling low were converted from a word answer (never, sometimes, most days, every day) to a numerical value (0, 1, 2, 3).
d) Combined symptom scores were produced by adding the values for specific combinations of symptoms to produce composite scores. The composite scores were then binned.
c) Questions regarding the subject's family history of depression/anxiety were converted from word answers (none, secondary relatives only, primary relatives) to numerical values (0, 1, 2).
f) Questions regarding the subject's personal history of depression/anxiety or pharmacological treatments for depression/anxiety were converted from word answers (none, one or more) into a binary response (0, 1).

After coding, various statistical tests, including Spearman correlation analysis, t-tests and ANOVA, were used to search for associations between gene expression levels and specific clinical variables.

Using statistical tests, as appropriate, the expression of each gene was compared to the coded answers provided by the subjects on the self-assessed questionnaire to identify correlations. Since a total of 377 comparisons were made (29 genes times 13 questionnaire responses), the threshold for significance was set at p<0.01 to minimize the possibility of Type 1 errors, while still retaining a large number of statistically significant results.

Tables 3A and 3B show correlation data for only 15 of the 29 genes (from Table 1A) that have significant differences within the control population based on the questionnaire responses analyzed. No significant differences were detected for the remaining genes. Tables 3A and 3B show data for 11 of the 13 questionnaire responses, however correlation data for BMI and age are not shown, as they were not significantly different. Some of the clinical parameters that correlate with significant gene expression profiles are lifetime experiences, lifetime treatments, and symptom scores.

TABLE 3A

| | CREB2 | DPP4 | ERK1 | ERK2 | GR | Gs | MAPK8 | MAPK14 |
|---|---|---|---|---|---|---|---|---|
| 1) Family History | | Inc** | | | | | Inc** | |
| (D/A/S) | | | | | | | | |
| 2) Family History | | | | | | | | |
| (D/A/S) | | | | | | | | |
| 1) Tobacco use | | | | | | | | |
| 2) Tobacco use | | | | | | | | |
| 1) Lifetime | Inc*** | Inc*** | | Inc** | Inc*** | | Inc*** | |
| experiences (D/A) | | | | | | | | |
| 2) Lifetime | | Inc*** | | | trend | | | |
| experiences (D/A) | | | | | up | | | |
| 1) Lifetime | Inc** | Inc*** | | | Inc** | | Inc*** | |
| treatments (D/A) | | | | | | | | |
| 2) Lifetime | | | | | trend | Inc** | | |
| treatments (D/A) | | | | | up | | | |
| 1) Appetite Change | | Inc** | | | | | | |
| 2) Appetite Change | | | | | | | | |
| 1) Sleep Problems | | Inc** | | | | | | |
| 2) Sleep Problems | | Inc** | | | | | | |
| 1) 10 Symptom | | Inc*** | | | | | Inc*** | |
| score (*) | | | | | | | | |
| 2) 10 Symptom | | | Inc** | Inc*** | | | | |
| score (*) | | | | | | | | |
| 1) Vegetative | | Inc** | | | | | | |
| symptoms | | | | | | | | |
| 2) Vegetative | | | | | | | | |
| symptoms | | | | | | | | |
| 1) Recent stress | | Inc** | | | | | | |
| 2) Recent stress | | | | | | | | |
| 1) Early life stress | | | | | | | | |
| 2) Early life stress | | | | | | | | |
| 1) Interest in sex | | | | | Inc** | | | |
| 2) Interest in sex | | | | | | | | Inc** |

1) US subjects
2) DK subjects
(D/A/S = Depression/Anxiety/Suicide; D/A = Depression/Anxiety)

TABLE 3B

| | | | | | S100 | | |
|---|---|---|---|---|---|---|---|
| | MKP1 | MR | PBR | RGS2 | A10 | SERT | VMAT2 |
| 1) Family History | | | | | | | |
| (D/A/S) | | | | | | | |
| 2) Family History | | | | | | | |
| (D/A/S) | | | | | | | |
| 1) Tobacco use | | | | | | Dec*** | |
| 2) Tobacco use | | | | | | trend | |
| | | | | | | down | |
| 1) Lifetime | | | | Inc** | | | Inc ** |
| experiences (D/A) | | | | | | | |
| 2) Lifetime | | | | trend | | | |
| experiences (D/A) | | | | up | | | |
| 1) Lifetime | Inc** | | | | | | |
| treatments (D/A) | | | | | | | |
| 2) Lifetime | trend | | | | | | |
| treatments (D/A) | up | | | | | | |
| 1) Appetite Change | | | | | | Dec** | |
| 2) Appetite Change | | | | | | trend | |
| | | | | | | down | |
| 1) Sleep Problems | | | | | | | |
| 2) Sleep Problems | | | | | | | |
| 1) 10 Symptom | trend | | Dec** | | | | |
| score (*) | up | | | | | | |
| 2) 10 Symptom | Inc** | | Inc** | | | | |
| score (*) | | | | | | | |
| 1) Vegetative | | | | | | | |
| symptoms | | | | | | | |
| 2) Vegetative | | | | | | | |
| symptoms | | | | | | | |
| 1) Recent stress | | | | | | | |
| 2) Recent stress | | | | | | | |
| 1) Early life stress | | | | | | | |
| 2) Early life stress | | | | | Inc*** | | |
| 1) Interest in sex | | | trend | | | | |
| | | | down | | | | |
| 2) Interest in sex | | Dec** | Inc** | | | | |

1) US subjects
2) DK subjects
(D/A/S = Depression/Anxiety/Suicide; D/A = Depression/Anxiety)

Of the 377 total combinations that were analyzed, twenty-three combinations (6%) indicate significant differences between the two control groups analyzed. However, three hundred forty-five (94%) of the combinations exhibit the same profile. Nine of the these combinations display changes in gene expression in the same direction (i.e. up- or down-regulation of genes) for both control groups studied, as indicated by the shaded boxes in Tables 3A and 3B. Overall, the analysis shows that the two control groups used for analysis are displaying very similar gene expression trends or profiles.

Gene expression profiles related to clinical parameters may also be analyzed by the multivariate algorithms described herein. Accordingly, clinical variables combined with transcription data may be subjected to any suitable algorithm known to those skilled in the art, such as Stepwise Logistic Regression or PELORA.

### Identification of Transcription Profiles in Depressed Patients.

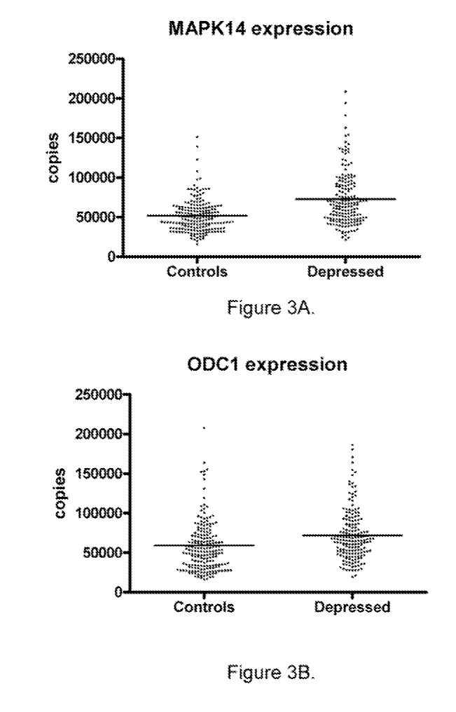

Blood samples obtained from 174 moderately depressed patients/subjects not receiving antidepressant treatment were first analyzed by univariate methods. Transcription levels for genes selected from Table 1A were measured and compared to the expression levels of such genes in 196 healthy control subjects. The expression profiles of representative genes in depressed patients as compared to controls are shown in FIGS. 2A-2B and 3A-3B.

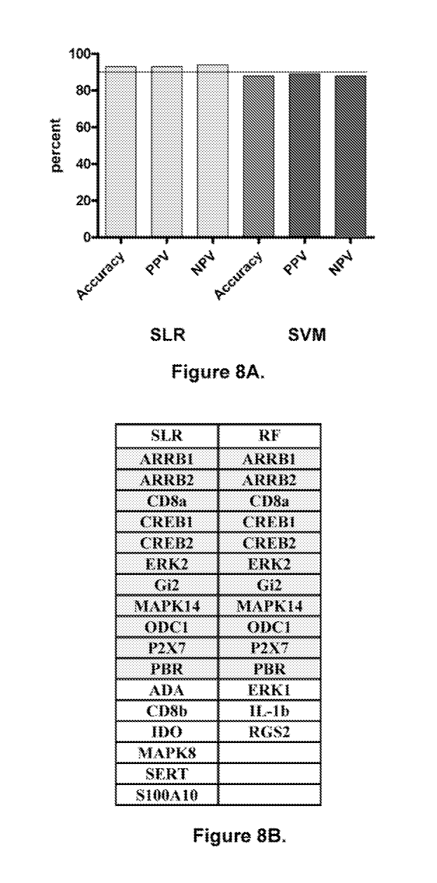

Classification of the moderately depressed patients v. controls using RF (selection) and SVM (training) resulted in a high accuracy of 88% as shown in FIG. 8A (PPV=89%; NPV=88%). Classification of the moderately depressed patients v. controls using an SLR algorithm, which performs both the gene selection and training, resulted in a high accuracy of 93% as shown in FIG. 8A (PPV=93%; NPV=94%).

Both algorithms exhibited good agreement in the genes selected based on the entire data set as shown in FIG. 8B. Random Forest selected 14 genes and SLR selected 17 genes as the most important genes for classification based on the statistical parameters of each method. Eleven genes were selected by both methods, including ARRB1, ARRB2, CD8a, CREB1, CREB2, ERK2, Gi2, MAPK14, ODC1, P2X7, and PBR.

Data sets were randomized, i.e. the assignments of samples as patient or control are randomized, and subjected to the same multivariate analysis as above. Following randomization, both classification algorithms (RF/SVM and SLR) produced accuracy values that are statistically different from those obtained with the actual data, indicating that the values listed above (FIG. 8A) are better than chance and the groups are statistically separable.

Subjects may be profiled and their transcription data based on the genes in Table 1A subjected to the classification algorithms trained with the parameters as described hereinabove to obtain a diagnosis of moderate depression.

Transcriptional profiles of depressed subjects for genes selected from Table 1A are shown in Table 4 based on abundance of each biomarker (i,e, gene transcript). Control subject transcript values are shown for comparison.

TABLE 4

| | Depressed Subject | Control Subject |
|---|---|---|
| | group features: | group features: |
| Biomarker | Abundance = Mean | Abundance = Mean |
| (Gene | transcript value of | transcript value of |
| abbreviation) | Biomarker (±SD) | Biomarker (±SD) |
| ADA | 4691 ± 2453 | 4511 ± 1710 |
| ARRB1 | 189062 ± 62727 | 297143 ± 91094 |
| ARRB2 | 84195 ± 31728 | 114780 ± 39962 |
| CD8a | 8304 ± 5825 | 14693 ± 8416 |
| CD8b | 8145 ± 4394 | 8687 ± 3880 |
| CREB1 | 71743 ± 20237 | 63725 ± 16022 |
| CREB2 | 63732 ± 14463 | 77059 ± 15755 |
| DPP4 | 6649 ± 2331 | 7169 ± 2890 |
| ERK1 | 25326 ± 10178 | 39016 ± 12900 |
| ERK2 | 58338 ± 18813 | 54137 ± 18660 |
| Gi2 | 115117 ± 53383 | 226358 ± 87609 |
| Gs | 262885 ± 112989 | 303930 ± 139837 |
| GR | 73224 ± 23517 | 80610 ± 26544 |
| IL1b | 29631 ± 13692 | 21006 ± 9313 |
| IL6 | 348 ± 523 | 182 ± 221 |
| IL8 | 45487 ± 106224 | 28024 ± 19993 |
| INDO | 6031 ± 10133 | 5596 ± 4418 |
| MAPK14 | 73156 ± 33915 | 51632 ± 20341 |
| MAPK8 | 12906 ± 3836 | 12162 ± 3500 |
| MKP1 | 525383 ± 268053 | 499308 ± 220665 |
| MR | 2565 ± 1110 | 2830 ± 887 |
| ODC1 | 71892 ± 32249 | 58670 ± 40801 |
| P2X7 | 1095 ± 432 | 1542 ± 563 |
| PBR | 70854 ± 30278 | 64439 ± 29328 |
| PREP | 6715 ± 2072 | 7072 ± 2102 |
| RGS2 | 632976 ± 262593 | 477280 ± 165907 |
| S100A10 | 32173 ± 9530 | 35819 ± 10568 |
| SERT | 1400 ±1164 | 1711 ± 1317 |
| VMAT2 | 3469 ± 1602 | 2792 ±1344 |

(SD = standard deviation)

Two-gene combinations were also evaluated by comparing the ratio of transcript values for depressed subjects vs. control subjects. Marked differences in the ratio of abundance of certain biomarkers are seen between depressed subjects and control subjects as in Table 4A.

TABLE 4A

| | Ratio of abundance of | Ratio of abundance of |
|---|---|---|
| | transcript for Depressed | transcript for Control |
| Biomarker | Subject group | group |
| ERK1 | 0.35 | 0.76 |
| MAPK14 | | |
| IL1b | 0.26 | 0.09 |
| Gi2 | | |
| MAPK14 | 0.39 | 0.17 |
| ARRB1 | | |
| ERK1 | 0.85 | 1.86 |
| IL1b | | |

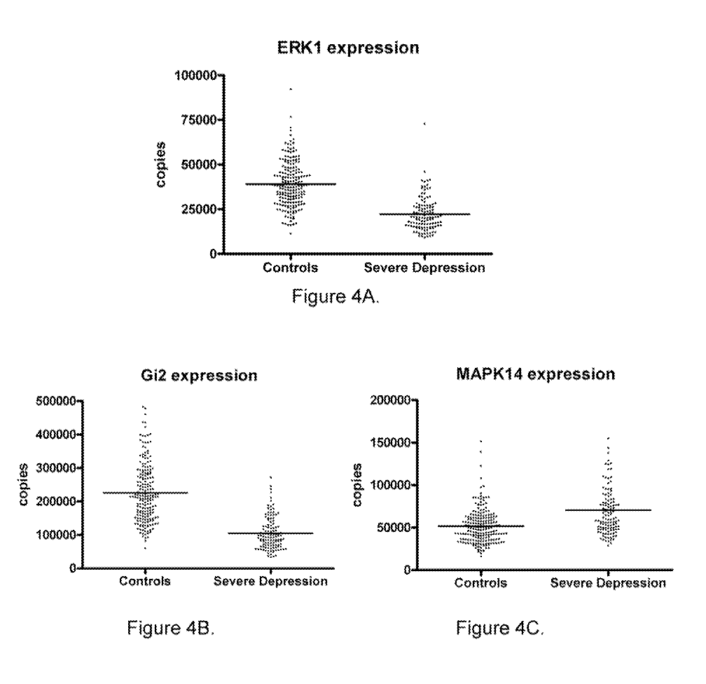

To assess the changes in transcription profiles in a more severely depressed patient population, blood from 120 severely depressed patients was obtained and gene expression measured for genes selected from Table 1A. Gene expression data was statistically analyzed by univariate methods. Patient transcription data was compared to that of 196 controls and representative scatter plots for individual gene data are shown in FIGS. 4A-4C.

Classification using RF/SVM resulted in a high accuracy of 92% (PPV=89%; NPV=94%). Classification of an SLR algorithm, which performs both the gene selection and training, resulted in a high accuracy of 93% (PPV=91%; NPV=95%).

Both algorithms showed good agreement in the genes selected based on the entire data set. A Random Forest classification selected 7 total genes and SLR selected 12 total genes as the most important genes for classification based on the statistical parameters of each method. Five genes were selected by both methods, including CD8a, ERK1, MAPK14, P2X7, and PBR.

Following a randomization of patient/control assignments, both classification algorithms (RF/SVM and SLR) produced accuracy values that are statistically different from those obtained with the actual data, indicating that the values listed above are better than chance and the groups are statistically separable.

Subjects may be profiled and their transcription data, based on the genes included in Table 1A, subjected to the classification algorithms trained as described hereinabove to obtain a diagnosis of severe depression.

Transcriptional profiles of severely depressed subjects for genes selected from Table 1A are shown in Table 5 based on abundance of each biomarker (i,e, gene transcript). Control subject transcript values are shown for comparison.

TABLE 5

| | Severely Depressed | |
|---|---|---|
| | Subject group | Control Subject |
| | features: | group features: |
| Biomarker | Abundance = Mean | Abundance = Mean |
| (Gene | transcript value of | transcript value of |
| abbreviation) | Biomarker (±SD) | Biomarker (±SD) |
| ADA | 3812 ± 1365 | 4511 ± 1710 |
| ARRB1 | 161284 ± 47341 | 297143 ± 91094 |
| ARRB2 | 79487 ± 22860 | 114780 ± 39962 |
| CD8a | 7666 ± 4603 | 14693 ± 8416 |
| CD8b | 6897 ± 3320 | 8687 ± 3880 |
| CREB1 | 64463 ± 18736 | 63725 ± 16022 |
| CREB2 | 71534 ± 12311 | 77059 ± 15755 |
| DPP4 | 5873 ± 2194 | 7169 ± 2890 |
| ERK1 | 19389 ± 7612 | 39016 ± 12900 |
| ERK2 | 48236 ± 17894 | 54137 ± 18660 |
| Gi2 | 97344 ± 42195 | 226358 ± 87609 |
| Gs | 185642 ± 82731 | 303930 ± 139837 |
| GR | 75411 ± 24542 | 80610 ± 26544 |
| IL1b | 27643 ± 12046 | 21006 ± 9313 |
| IL6 | 153 ± 100 | 182 ± 221 |
| IL8 | 38817 ± 29253 | 28024 ± 19993 |
| INDO | 5735 ± 5467 | 5596 ± 4418 |
| MAPK14 | 67519 ± 29094 | 51632 ± 20341 |
| MAPK8 | 11446 ± 3231 | 12162 ± 3500 |
| MKP1 | 615915 ± 307961 | 499308 ± 220665 |
| MR | 2023 ± 893 | 2830 ± 887 |
| ODC1 | 55085 ± 30043 | 58670 ± 40801 |
| P2X7 | 769 ± 331 | 1542 ± 563 |
| PBR | 67863 ± 24974 | 64439 ± 29328 |
| PREP | 5186 ± 1620 | 7072 ± 2102 |
| RGS2 | 571284 ± 270572 | 477280 ± 165907 |
| S100A10 | 21812 ± 7985 | 35819 ± 10568 |
| SERT | 795 ± 553 | 1711 ± 1317 |
| VMAT2 | 3073 ± 1715 | 2792 ± 1344 |

(SD = standard deviation)

Genes for which the mean expression levels (transcript values) were significantly different (p0.05) between severely depressed patients and controls are: ADA, ARRB1, ARRB2, CD8a, CD8b, CREB2, DPP4, ERK1, Gi2, Gs, IL1b, IL8, MAPK14, MKP1, MR, P2X7, PREP, RGS2, S100A10, and SERT (Table 5A).

TABLE 5A
Genes that are significantly different in severely depressed subjects
as compared to control subjects, based on p-values (p < 0.05).

| Biomarker | |
|---|---|
| (Gene | |
| abbreviation) | p-value |
| ADA | 3.2673 × 10⁻⁶ |
| ARRB1 | 4.40419 × 10⁻⁶⁰ |
| ARRB2 | 1.61434 × 10²⁷ |
| CD8a | 1.92916 × 10³⁸ |
| CD8b | 3.13307 × 10⁸ |
| CREB2 | 0.0000507671 |
| DPP4 | 1.25015 × 10⁷ |
| ERK1 | 1.12946 × 10⁻⁷² |
| Gi2 | 3.27538 × 10⁻⁶⁴ |
| Gs | 1.98625 × 10³⁵ |
| IL1b | 2.13924 × 10⁻¹¹ |
| IL8 | 2.00073 × 10⁻⁶ |
| MAPK14 | 5.2042 × 10⁻¹⁵ |
| MKP1 | 1.25421 × 10⁻⁶ |
| MR | 1.73784 × 10⁻²³ |
| P2X7 | 3.7121 × 10⁻⁶⁷ |
| PREP | 2.72022 × 10⁻²⁶ |
| RGS2 | 0.0000152985 |
| S100A10 | 2.3756 × 10⁻⁵³ |
| SERT | 4.36216 × 10⁻²⁶ |

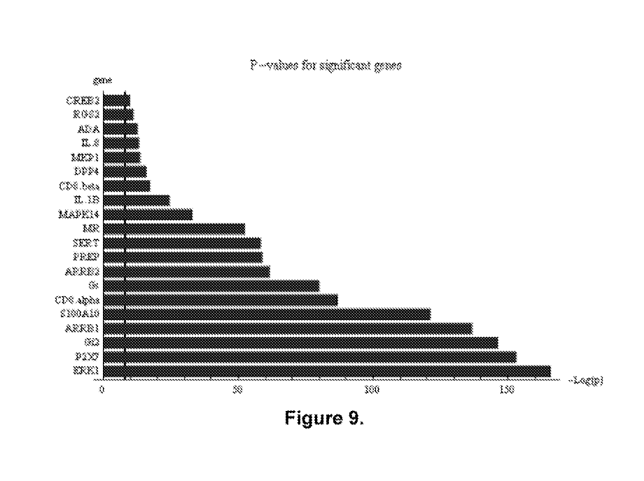

These genes were ranked according to the magnitude of the calculated −Log(p) value (FIG. 9), thereby indicating the marked differences between patient transcript value and control value for several genes, such as ERK1, P2X7, Gi2, ARRB1 and S100A10.

In order to search for linear and non-linear interactions between transcript values the relevance vector machine (RVM) classifying algorithm was performed, then a Genetic algorithm was used in order to search through the space of possible gene-gene interactions and select the most robust and meaningful interactions. Single-gene solutions were also examined by this set of algorithms, and confirms the validity of single-gene solutions for separating patients from controls. ARRB1 (accuracy=0.86) and ERK1 (accuracy=0.85) are determined to be highly informative in a single-gene analysis, followed by P2X7 (accuracy=0.82) and Gi2 (accuracy=0.89. See also, for example, FIGS. 2 through 5 wherein informative gene expression data is depicted for moderately depressed, severely depressed and bipolar patients vs. controls.

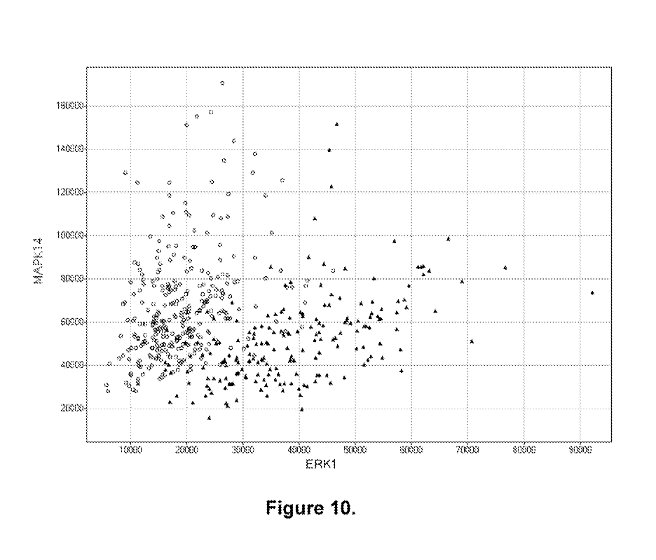

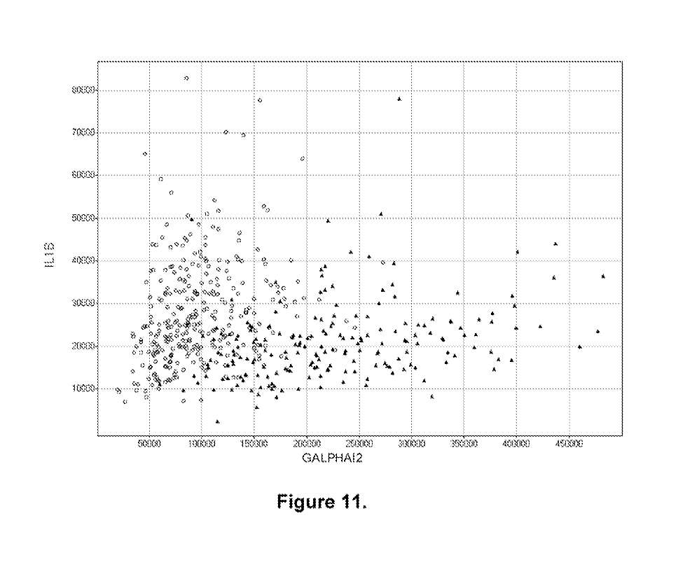

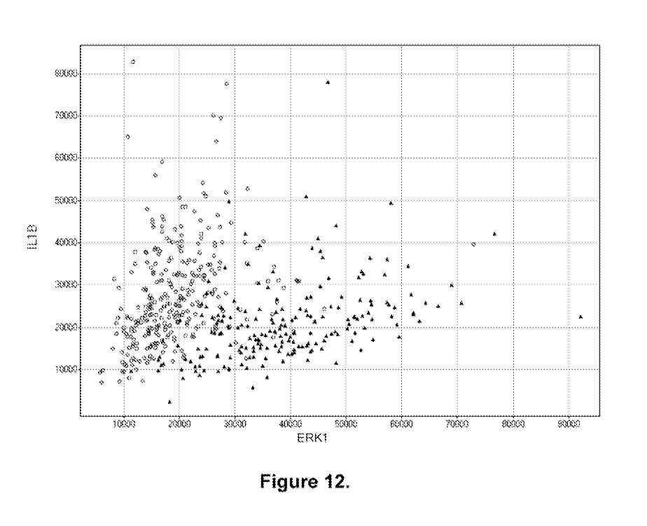

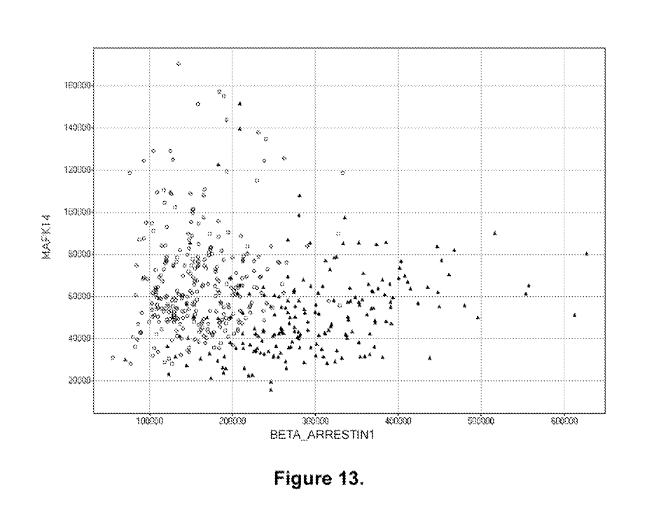

Several two-gene solutions have been identified for classifying depressed patients and controls with 90% or greater accuracy. ERK1 and MAPK14 transcript values are shown to classify a depressed patient, vs. control, with an accuracy of 92%. FIG. 10 depicts the distribution of severely depressed subjects and controls based solely on the transcript values of ERK1 and MAPK14. The classification of depressed subjects (with profiles as in Table 4) is consistent with the results of severely depressed Subjects. FIGS. 11, 12 and 13 depict the distribution of severely depressed subjects and controls based on the transcript values of other two-gene transcription profiles, IL1b/Gi2, MAPK14/ARRB1, and ERK1/IL1b, respectively. Two-gene combinations were also evaluated by comparing the ratio of transcript values for severely depressed subjects vs. control subjects. Marked differences in the ratio of abundance between severely depressed subjects and control subjects are seen in Table 5B.

TABLE 5B

| | Ratio of abundance of | Ratio of abundance of |
|---|---|---|
| | transcript for Severely | transcript for Control |
| Biomarker | Depressed Subject group | group |
| ERK1 | 0.29 | 0.76 |
| MAPK14 | | |
| IL1b | 0.28 | 0.09 |
| Gi2 | | |
| MAPK14 | 0.42 | 0.17 |
| ARRB1 | | |
| ERK1 | 0.70 | 1.86 |
| IL1b | | |

Identification of Transcription Profiles in Patients with Bipolar Disorder.

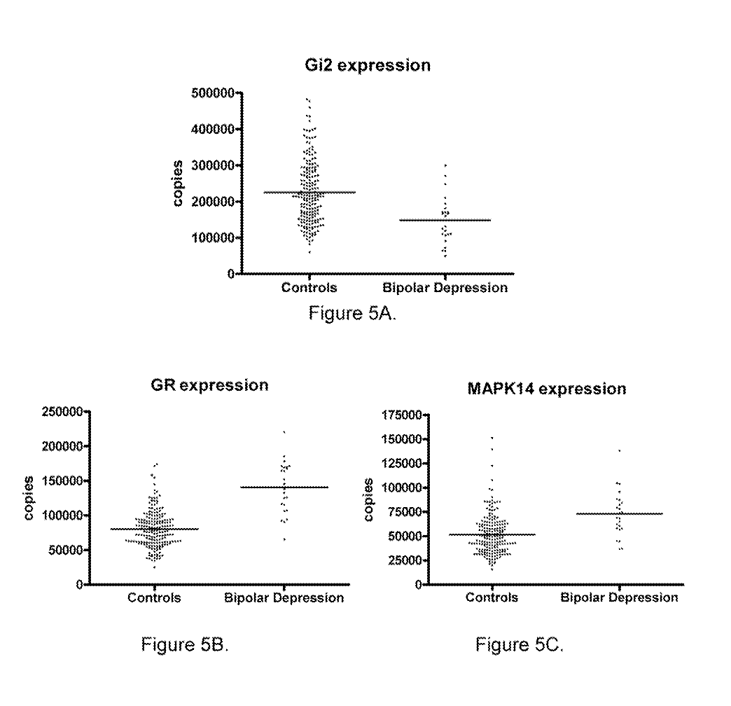

To assess the changes in transcription profiles in patients with bipolar disorder, blood from 23 depressed patients (20 patients being definitively diagnosed with bipolar disorder according to the DSM-IV criteria) was obtained and gene expression measured for genes selected from Table 1A. Gene expression data was statistically analyzed by univariate methods. Patient transcription data was compared to that of 196 controls and representative scatter plots for individual gene data are shown in FIGS. 5A-5C.

Classification using RF/SVM resulted in a high accuracy of 94% (PPV=86%; NPV=95%). Classification of an SLR algorithm, which performs both the gene selection and training, resulted in a high accuracy of 97% (PPV=90%; NPV=99%).

Both algorithms showed good agreement in the genes selected based on the entire data set, with a Random Forest classification selecting 3 total genes and SLR selecting 5 total genes as the most important genes for classification based on the statistical parameters of each method. Three genes were selected by both methods, including Gi2, GR, and MAPK14.

Following a randomization of patient/control assignments, both classification algorithms (RF/SVM and SLR) produced accuracy values that are statistically different from those obtained with the actual data, indicating that the values listed above are better than chance and the groups are statistically separable.

Subjects may be profiled and their transcription data, based on the genes included in Table 1A, subjected to the classification algorithms trained as described hereinabove to obtain a diagnosis of bipolar disorder.

Transcriptional profiles of bipolar subjects for each gene are shown in Table 6 based on abundance of each biomarker (i,e, gene transcript). Control subject transcript values are shown for comparison.

TABLE 6

| | Bipolar Subject | Control Subject |
|---|---|---|
| | group features: | group features: |
| Biomarker | Abundance = Mean | Abundance = Mean |
| (Gene | transcript value of | transcript value of |
| abbreviation) | Biomarker (±SD) | Biomarke (±SD) |
| ADA | 4775 ± 1508 | 4511 ± 1710 |
| ARRB1 | 292298 ± 89272 | 297143 ± 91094 |
| ARRB2 | 111023 ± 39397 | 114780 ± 39962 |
| CD8a | 11668 ± 5573 | 14693 ± 8416 |
| CD8b | 7998 ± 3841 | 8687 ± 3880 |
| CREB1 | 62347 ± 18282 | 63725 ± 16022 |
| CREB2 | 79456 ± 16778 | 77059 ± 15755 |
| DPP4 | 7618 ± 3077 | 7169 ± 2890 |
| ERK1 | 34901 ± 15116 | 39016 ± 12900 |
| ERK2 | 57832 ± 21427 | 54137 ± 18660 |
| Gi2 | 192417 ± 98987 | 226358 ± 87609 |
| Gs | 304202 ± 171505 | 303930 ± 139837 |
| GR | 124054 ± 42231 | 80610 ± 26544 |
| IL1b | 21577 ± 13468 | 21006 ± 9313 |
| IL6 | 173 ± 78 | 182 ± 221 |
| IL8 | 24568 ± 19226 | 28024 ± 19993 |
| INDO | 5428 ± 3847 | 5596 ± 4418 |
| MAPK14 | 66946 ± 25751 | 51632 ± 20341 |
| MAPK8 | 12584 ± 3060 | 12162 ± 3500 |
| MKP1 | 501068 ± 251853 | 499308 ± 220665 |
| MR | 3409 ± 1094 | 2830 ± 887 |
| ODC1 | 67672 ± 50925 | 58670 ± 40801 |
| P2X7 | 1322 ± 418 | 1542 ± 563 |
| PBR | 64761 ± 29660 | 64439 ± 29328 |
| PREP | 6806 ± 1677 | 7072 ± 2102 |
| RGS2 | 499864 ± 264854 | 477280 ± 165907 |
| S100A10 | 42063 ± 12765 | 35819 ± 10568 |
| SERT | 1435 ± 710 | 1711 ± 1317 |
| VMAT2 | 2736 ± 1050 | 2792 ± 1344 |

(SD = standard deviation)

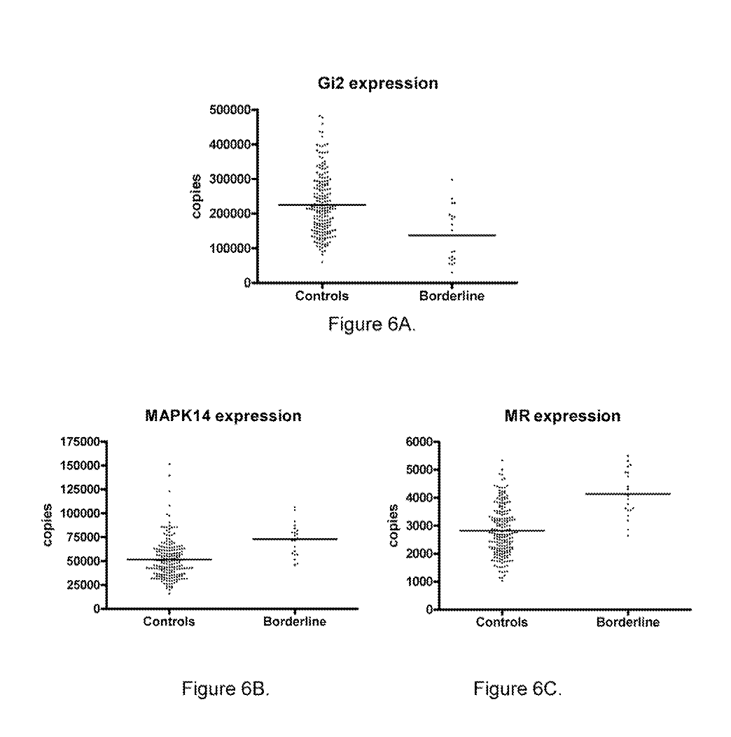

Identification of transcription profiles in patients with borderline personality disorder. To assess the changes in transcription profiles in patients with borderline personality disorder, blood from 21 borderline personality disorder patients was obtained and gene expression measured for genes selected from Table 1A. Gene expression data was statistically analyzed by univariate methods. Patient transcription data was compared to that of 196 controls and representative scatter plots for individual gene data are shown in FIGS. 6A-6C.

Classification using RF (selection) and SVM (training) resulted in a high accuracy of 97% (PPV=87%; NPV=98%). Classification of an SLR algorithm, which performs both the gene selection and training, resulted in a high accuracy of 98% (PPV=90%; NPV=100%).

Both algorithms showed good agreement in the genes selected based on the entire data set, with a Random Forest classification selecting 5 total genes and SLR selecting 4 total genes as the most important genes for classification based on the statistical parameters of each method. Four genes were selected by both methods, including Gi2, GR, MAPK14, and MR.

Following a randomization of patient/control assignments, both classification algorithms (RF/SVM and SLR) produced accuracy values that are statistically different from those obtained with the actual data, indicating that the values listed above are better than chance and the groups are statistically separable.

Subjects may be profiled and their transcription data, based on the genes included in Table 1A, subjected to the classification algorithms trained as described hereinabove to obtain a diagnosis of borderline personality disorder.

Identification of Transcription Profiles in Patients with PTSD.

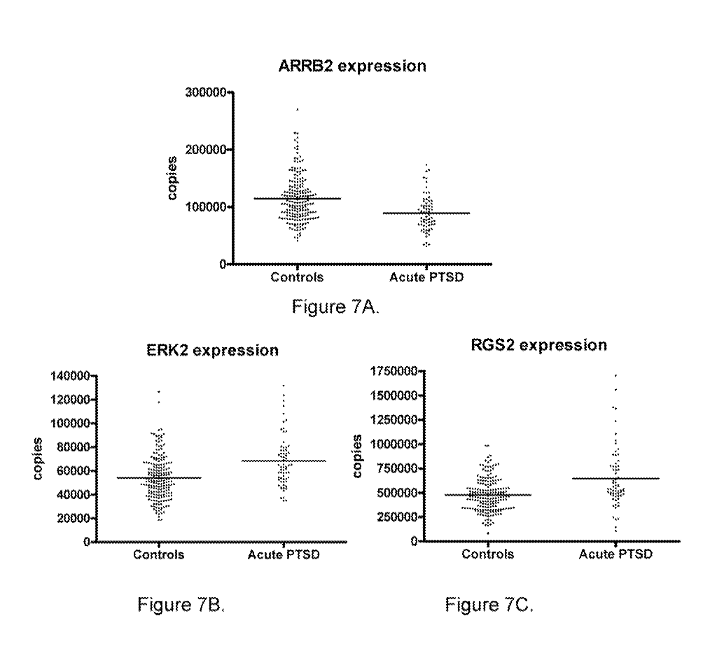

Transcription profiles were assessed in patients with acute PTSD, patients with remitted PTSD, and a group of individuals who had been subjected to traumatic events without developing PTSD. The combined evaluation of these groups presents the opportunity to identify expression changes related to acute PTSD as well as to define differences that may correlate with recovery from or resistance to the disease. Gene expression data was statistically analyzed by univariate methods. Patient transcription data from 66 patients with acute PTSD was compared to that of 196 controls and representative scatter plots for individual gene data are shown in FIGS. 7A-7C.

Classification of acute PTSD patients compared to control subjects using RF (selection) and SVM (training) resulted in an accuracy of 77% (PPV=64%; NPV=82%). Classification with an SLR algorithm, which performs both the gene selection and training, resulted in an accuracy of 84% (PPV=77%; NPV=87%). The SLR algorithm outperforms the SVM algorithm using this set of test data. Each classification algorithm was compared with randomized (permuted) versions of the data sets and SLR produced an accuracy value of 73% (PPV=39%; NPV=75%) using the permuted data sets. Statistical analysis indicated that the SLR accuracy values obtained with the real versus randomized data are different, indicating that the groups are separable.

Using the permuted data sets, SVM produced an accuracy value of 73% (PPV=10%; NPV=75%), indicating a trend downward for the permuted (randomized) data. It is noted that PPV (ability to positively predict patients with the disease) using the real data in the SVM algorithm is better than 60%, compared to 10% precision with the permuted data, indicating that the algorithm trained using the real data outperforms random prediction.

SLR selected 10 total genes as the most important genes for classification based on the entire data set of acute PTSD patients v. controls: ARRB1, ARRB2, CD8b, ERK2, IDO, IL-6, MR, ODC1, PREP and RGS2.

Subjects may be profiled and their transcription data, based on the genes included in Table 1A, subjected to the classification algorithms trained as described hereinabove to obtain a diagnosis of acute PTSD.

Classification of remitted PTSD patients compared to control subjects using RF (selection) and SVM (training) resulted in an accuracy of 81% (PPV=59%; NPV=85%). Classification of an SLR algorithm, which performs both the gene selection and training, resulted in an accuracy of 80% (PPV=33%; NPV=86%). However, when the classification algorithm was run on the randomized versions of this data set, SVM and SLR produced accuracy values of 82% and 81%, respectively. These values are not statistically different from those obtained with the real data, indicating that the algorithms cannot reliably separate these groups. Because of the lack of separation, a gene list is not reported for this comparison. From a clinical perspective, the inability of the algorithms to distinguish between the controls and the remitted patients is expected due to the lack of biological differences between these groups. As the remitted patients no longer exhibit symptoms of the illness, it is reasonable to assume that their gene expression levels have returned to normal levels, thereby preventing the algorithms from effectively separating the groups.

Classification of subjects who were traumatized but did not develop PTSD compared to control subjects using RF (selection) and SVM (training) resulted in an accuracy of 74% (PPV=61%; NPV=79%). Classification of an SLR algorithm, which performs both the gene selection and training, resulted in an accuracy of 73% (PPV=59%; NPV=80%). When the multivariate analysis was performed on randomized data sets, both RF/SVM and SLR classification algorithms produced accuracy values that are statistically different from those obtained with the actual data, indicating the values as reported above are better than chance and the groups are separable.

The Random Forest classification selected 14 total genes and SLR selected 13 total genes as the most important genes for classification based on the statistical parameters of each method and using the entire data set from trauma patients and controls. Seven genes were selected by both methods, including ARRB2, CREB1, ERK2, Gs, IL-6, MKP1, and RGS2.

Although these individuals are not diagnosed with PTSD, the algorithms can still distinguish them from controls, albeit with lower accuracy, PPV, and NPV values than for some of the other comparisons presented herein. Interestingly, 6 of the genes on the SLR gene list from the acute PTSD patients match those on the corresponding list for the trauma without PTSD patients (ARRB2, CD8b, ERK2, MR, IL-6, and RGS2). While the traumatized patients have not yet developed the illness, they share some gene expression profiles with patients who have, indicating that they may be at risk.

Subjects may be profiled and their transcription data, based on the genes included in Table 1A, subjected to the classification algorithms trained as described hereinabove to obtain a diagnosis of trauma without PTSD.

### 7 REFERENCES CITED

All references cited herein are incorporated herein by reference in their entirety and for all purposes to the same extent as if each individual publication or patent or patent application was specifically and individually indicated to be incorporated by reference in its entirety herein for all purposes.

### 8 MODIFICATIONS

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of diagnosing an affective disorder in a test subject, the method comprising:
evaluating whether a plurality of features of a plurality of biomarkers in a biomarker profile of the test subject satisfies a value set, wherein satisfying the value set predicts that the test subject has said affective disorder, and wherein the plurality of features are measurable aspects of the plurality of biomarkers, the plurality of biomarkers comprising at least two biomarkers listed in Table 1A.

2. The method of claim 1, the method further comprising outputting a diagnosis of whether the test subject has the affective disorder to a user interface device, a monitor, a tangible computer readable storage medium, or a local or remote computer system; or displaying a diagnosis of whether the test subject has the affective disorder in user readable form.

3. The method of claim 1, wherein said plurality of biomarkers consists of between 2 and 29 biomarkers listed in Table 1A.

4. The method of claim 1, wherein said plurality of biomarkers consists of between 3 and 20 biomarkers listed in Table 1A.

5. (canceled)

6. The method of claim 1, wherein said plurality of biomarkers comprises at least three biomarkers listed in Table 1A.

7. The method of claim 1, wherein said plurality of biomarkers comprises at least four biomarkers listed in Table 1A.

8-10. (canceled)

11. The method of claim 1, wherein said plurality of biomarkers comprises ERK1 and MAPK14.

12. The method of claim 1, wherein said plurality of biomarkers comprises Gi2 and IL-1b.

13. The method of claim 1, wherein said plurality of biomarkers comprises ARRB1 and MAPK14.

14. The method of claim 1, wherein said plurality of biomarkers comprises ERK1 and IL1b.

15. The method of claim 1, wherein said plurality of biomarkers comprises ARRB1, IL6 and CD8a.

16. The method of claim 1, wherein said plurality of biomarkers comprises ARRB1, ODC1 and P2X7.

17. The method of claim 1, wherein each biomarker in said plurality of biomarkers is a nucleic acid.

18. The method of claim 1, wherein each biomarker is in said plurality of biomarkers is a DNA, a cDNA, an amplified DNA, an RNA, or an mRNA.

19. The method of claim 1, wherein a feature in said plurality of features in the biomarker profile of the test subject is a measurable aspect of a biomarker in the plurality of biomarkers and a feature value for said feature is determined using a biological sample taken from said test subject.

20. The method of claim 19, wherein said feature is abundance of said biomarker in the biological sample, and the biological sample is whole blood.

21. The method of claim 1, the method further comprising constructing, prior to the evaluating step, said first value set.

22. The method of claim 21, wherein the constructing step comprises applying a data analysis algorithm to features obtained from members of a population.

23. The method of claim 22, wherein said population comprises a first plurality of biological samples from a first plurality of control subjects not having the affective disorder and a second plurality of biological samples from a second plurality of subjects having the affective disorder.

24. The method of claim 22, wherein said data analysis algorithm is a decision tree, predictive analysis of microarrays, a multiple additive regression tree, a neural network, a clustering algorithm, principal component analysis, a nearest neighbor analysis, a linear discriminant analysis, a quadratic discriminant analysis, a support vector machine, an evolutionary method, a relevance vector machine, a genetic algorithm, a projection pursuit, or weighted voting.

25. The method of claim 21, wherein the constructing step generates a decision rule and wherein said evaluating step comprises applying said decision rule to the plurality of features in order to determine whether they satisfy the first value set.

26. The method of claim 25, wherein said decision rule classifies subjects in said population as (i) subjects that do not have the affective disorder and (ii) subjects that do have the affective disorder with an accuracy of seventy percent or greater.

27. The method of claim 25, wherein said decision rule classifies subjects in said population as (i) subjects that do not have the affective disorder and (ii) subjects that do have the affective disorder with an accuracy of ninety percent or greater.

28. The method of claim 1, wherein the affective disorder is bipolar disorder I, bipolar disorder II, a dysthymic disorder, a depressive disorder, mild depression, moderate depression, severe depression, atypical depression, melancholic depression, or a borderline personality disorder.

29. (canceled)

30. A computer program product, wherein the computer program product comprises a computer readable storage medium and a computer program mechanism embedded therein, the computer program mechanism comprising instructions for carrying out the method of claim 1.

31-35. (canceled)
